(19) United States
(12) Patent Application Publication
(10) Pub. No.: US 2022/0206018 A1
(43) Pub. Date: Jun. 30, 2022

(54) METHODS AND SYSTEMS FOR THE DIAGNOSIS AND TREATMENT OF SEX HORMONE DISORDERS

(71) Applicant: Function Promoting Therapies LLC, Weston, MA (US)

(72) Inventors: Ravi Jasuja, Quincy, MA (US); Shalender Bhasin, Weston, MA (US)

(73) Assignee: Function Promoting Therapies LLC, Weston, MA (US)

(21) Appl. No.: 17/497,652

(22) Filed: Oct. 8, 2021

Related U.S. Application Data
(63) Continuation-in-part of application No. 16/503,286, filed on Jul. 3, 2019.
Application No. 16/503,286 is a continuation of application No. 14/772,554, filed on Sep. 3, 2015, now Pat. No. 10,386,375.
(60) Provisional application No. 61/772,054, filed on Mar. 4, 2013.
(60) Provisional application No. 63/090,115, filed on Oct. 9, 2020.

(51) Int. Cl. G01N 33/74; G16H 20/10; H01J 49/00

(52) U.S. Cl. CPC G01N 33/743; G01N 2800/34; H01J 49/0027; G16H 20/10

(57) ABSTRACT

The technology described herein is directed to the diagnosis and treatment of sex hormone disorders and/or deficiencies, such as estrogen and/or testosterone disorders and/or deficiencies.

### CROSS-REFERENCE TO RELATED APPLICATIONS

This application is a continuation-in-part of U.S. application Ser. No. 16/503,286, filed Jul. 3, 2019, which is a continuation of U.S. application Ser. No. 14/772,554, filed Sep. 3, 2015, which is a national stage filing under 35 U.S.C. § 371 of international PCT application PCT/US2014/020223, filed Mar. 4, 2014, which claims priority to U.S. Provisional Application No. 61/772,054 filed on Mar. 4, 2013, the contents of each of which are incorporated herein by reference.

### TECHNICAL FIELD

The technology described herein relates to the diagnosis and treatment of sex hormone (androgen and estrogen) disorders (including deficiencies and/or excesses of sex hormones).

### BACKGROUND

Sex hormones (estrogen and testosterone) bind sex hormone binding globulin. Estradiol (E2), the dominant estrogen in men and women, is found in human circulation bound primarily to sex hormone-binding globulin (SHBG) and human serum albumin (HSA). These circulating binding proteins regulate the transport, bioavailability, and metabolism of estradiol, and the biological activity of the circulating hormone is related to the fraction that crosses into the tissue. Testosterone (T) is a primary androgenic hormone produced predominantly in the interstitial cells of the testes and is responsible for normal growth, development and maintenance of male sex organs and secondary sex characteristics (e.g., deepening voice, muscular development, facial hair, etc.). Throughout adult life, testosterone is necessary for proper functioning of the testes and its accessory structures, prostate and seminal vesicle; for sense of well-being; and for maintenance of libido, erectile potency, muscle mass, and bone health. Ovaries produce most of the circulating estrogens in females. In males, testes produce a small fraction of estrogens, with the generated by the conversion of testosterone by aromatase. Some fraction of estrogen is produced by adrenal glands in both men and women.

Testosterone deficiency is insufficient secretion of T characterized by low serum T concentrations and can give rise to medical conditions (e.g., hypogonadism) in males. Symptoms associated with male hypogonadism include impotence and decreased sexual desire, fatigue and loss of energy, low mood and depressive symptoms, regression of secondary sexual characteristics, decreased muscle mass, and increased fat mass. Furthermore, hypogonadism in men is a risk factor for anemia, osteoporosis, metabolic syndrome, type II diabetes and cardiovascular disease. Estrogen is primarily produced by ovaries in women and converted from testosterone in men. Small quantities are produced by adrenal glands. Estrogenic imbalance impact and/or are associated with numerous health conditions including but not necessarily limited to infertility, menopause, ovarian dysfunction, puberty, estrogen secreting tumors in men and women, and gynecomastia in men.

Circulating free testosterone (FT) levels have been used widely in the diagnosis and treatment of hypogonadism in men. Testosterone is the second most frequently ordered endocrine test. In 2012, nearly 4 million free testosterone tests were performed in the USA alone. A number of direct and indirect methods—equilibrium dialysis, ultrafiltration, tracer analog methods, and calculations based on homogenous SHBG:T binding equations—have been developed for the determination of FT levels. Due to experimental complexities in FT measurements, the Endocrine Society expert panel has recommended the use of calculated FT (cFT) as an appropriate approach for estimating FT. Expert panels have expressed concern about the accuracy and methodological complexity of the available assays for FT (Rosner et al 2007, Sodergard et al 1982, Vermeulen et al 1971). Similar concerns have existed for the determination of circulating free estrogen levels

### SUMMARY

Described herein is the demonstration that the prevailing model of sex hormone's binding to SHBG, including testosterone and estrogen's binding to SHBG, used in the current methods of testing and diagnosis is flawed, and their further discovery of an improved model that permits methods, assays, and systems with greater accuracy and reliability in measuring sex home levels, including testosterone and estrogen levels. The multi-step dynamic binding model with complex allostery described herein is a new model for calculation of free testosterone and estrogen. The multi-step dynamic binding model with complex allostery model is a modified ensemble allostery model that takes into consideration the specific SHBG-Sex Hormone binding interaction, including SHBG-Testosterone or SHBG-Estrogen binding interaction, described herein.

In one aspect, described herein is a computer implemented method for an assay, comprising: on a device having one or more processors and a memory storing one or more programs for execution by the one or more processors, the one or more programs including instructions for: a) receiving data from measuring i) a total SHBG concentration, ii) a total sex hormone concentration, for example a total estrogen or total testosterone concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer; and c) calculating the free sex hormone concentration, such as the free testosterone or free estrogen concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer.

In one aspect, described herein is a computer system for an assay, comprising: one or more processors; and memory to store: one or more programs, the one or more programs comprising: instructions for: a) receiving data from measuring i) a total SHBG concentration, ii) a total sex hormone concentration, such as total testosterone or total estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone, such as free testosterone or free estrogen concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer.

In one aspect, described herein is a non-transitory computer-readable storage medium storing one or more programs for treating an individual suspected of having a sex hormone disorder, including but not limited to an androgen disorder, the one or more programs for execution by one or more processors of a computer system, the one or more programs comprising instructions for: a) receiving data from measuring i) a total SHBG concentration, ii) a total sex hormone concentration, such as a total testosterone or total estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone concentration, such as the free testosterone or free estrogen concentration, in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer.

In one aspect, described herein is an assay comprising the steps of: a) measuring i) a total SHBG concentration, ii) a total sex hormone concentration, such as a total testosterone or total estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone concentration, such as free testosterone or free estrogen concentration, in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer

In some embodiments of any of the foregoing aspects, the step of attributing can be performed according to FIGS. 2, 3, 5, and 7. In some embodiments, of any of the foregoing aspects, the step of calculating can be performed according to FIG. 7 or Example 5. In some embodiments of any of the foregoing aspects, the assay, method, system, or medium can further comprise the step of determining the concentration of at least one steroid that is not the sex hormone being analyzed. In some embodiments, the steroid that is not the sex hormone being analyzed can be selected from the group consisting of an estradiol steroid, an estrone steroid, and a dihydrotestosterone steroid. In some embodiments of any of the foregoing aspects, the total SHBG concentration, the total sex hormone concentration, and the total albumin concentration is determined using an assay selected from the group consisting of an immunoassay, a binding assay, and a mass-spectrometry assay. In some embodiments of any of the foregoing aspects, instead of steps a-c, the data received is a previously calculated concentration of free sex hormone.

In one aspect, described herein is a computer implemented method for treating an individual suspected of having a sex hormone disorder, such as an androgen disorder, comprising: on a device having one or more processors and a memory storing one or more programs for execution by the one or more processors, the one or more programs including instructions for: a) receiving data from measuring i) a total SHBG concentration, ii) a total sex hormone concentration, such as a total testosterone or total estrogen concentration and iii) a total albumin concentration in a biological sample obtained from an individual suspected of having a sex hormone disorder, to determine free sex hormone concentration, such as free testosterone or free estrogen concentration, from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using a New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for administering a pharmaceutically effective amount of sex hormone to an individual having a free sex hormone concentration, such as a free testosterone or free estrogen concentration below the lower limit of a normal free sex hormone concentration from a healthy individual (e.g., in one embodiment, the lower limit of testosterone is 114.6 pg/ml); and e) sending a signal for not administering a pharmaceutically effective amount of sex hormone to an individual having a free sex hormone concentration above the lower limit of the normal free sex hormone concentration from a healthy individual (e.g., in one embodiment, the lower limit of testosterone is 114.6 pg/ml).

In one aspect, described herein is a computer system for treating an individual suspected of having a sex hormone disorder, comprising: one or more processors; and memory to store: one or more programs, the one or more programs comprising: instructions for: a) receiving data from measuring i) a total SHBG concentration, ii) a total sex hormone concentration, such as a testosterone or estrogen concentration, and iii) a total albumin concentration in a biological sample obtained from an individual suspected of having a sex hormone disorder, to determine free sex hormone, such as the free testosterone or free estrogen concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone, such as the free testosterone and/or free estrogen concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for administering to a pharmaceutically effective amount of sex hormone, such as testosterone or estrogen, to an individual having a free sex hormone concentration (based on a, b, c, d above or obtained otherwise) below the lower limit of a normal free sex hormone concentration from a healthy individual; and e) sending a signal for not administering a pharmaceutically effective amount of sex hormone to an individual having a free sex hormone concentration (based on a, b, c, d above or obtained otherwise) above the lower limit of the normal free sex hormone concentration from a healthy individual.

In one aspect, described herein is a non-transitory computer-readable storage medium storing one or more programs for treating an individual suspected of having a sex hormone disorder, the one or more programs for execution by one or more processors of a computer system, the one or more programs comprising instructions for: a) receiving data from measuring i) a total SHBG concentration, ii) a total sex hormone concentration, such as total testosterone and/or total estrogen concentration, and iii) a total albumin concentration in a biological sample obtained from an individual suspected of having a sex hormone disorder, to determine free sex hormone, such as free testosterone or free estrogen concentration, from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for administering a pharmaceutically effective amount of sex hormone, such as testosterone and/or estrogen, to an individual having a free sex hormone concentration below the lower limit of a normal sex hormone concentration from a healthy individual; and e) sending a signal for not administering a pharmaceutically effective amount of sex hormone, such as testosterone and/or estrogen, to an individual having a free sex hormone concentration above the lower limit of the normal free sex hormone concentration from a healthy individual.

In one aspect, described herein is a method for treating an individual suspected of having a sex hormone disorder comprising: a) administering a pharmaceutically effective amount of a sex hormone, such as testosterone and/or estrogen, to an individual who has had a free sex hormone level, such as a testosterone and/or an estrogen level, determined by measuring i) a total SHBG concentration, ii) a total sex hormone, such as a total testosterone and/or total estrogen concentration, and iii) a total albumin concentration in a biological sample from the individual suspected of having an androgen disorder; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using an the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) administering a pharmaceutically effective amount of sex hormone, such as testosterone or estrogen, to an individual having a free sex hormone concentration below the lower limit of a normal free sex hormone concentration from a healthy individual; and e) not administering a pharmaceutically effective amount of sex hormone to an individual having a free sex hormone concentration above the lower limit of the normal sex hormone concentration from a healthy individual.

In one aspect, described herein is a computer implemented method for determining a need for adjustment of a dose of sex hormone, such as testosterone and/or estrogen, administered to an individual with a sex hormone disorder, or any other condition for which sex hormone therapy, such as testosterone and/or estrogen therapy, is indicated, comprising: on a device having one or more processors and a memory storing one or more programs for execution by the one or more processors, the one or more programs including instructions for: a) receiving data from determining the concentration of free sex hormone, such as free testosterone and/or free estrogen in an individual receiving sex hormone therapy at a first dose, wherein the concentration of free sex hormone is determined by measuring i) a total SHBG concentration, ii) a total sex hormone concentration, such as total testosterone and/or total estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone, such as free testosterone and/or free estrogen concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer and; d) sending a signal for providing a second (adjusted) dose of sex hormone that is higher than the first dose when the free sex hormone concentration is below the lower end of the target therapeutic range; and e) sending a signal for providing a second (adjusted) dose of sex hormone that is lower than the first dose when the free sex hormone concentration is above the upper end of the target therapeutic range. In some embodiments, the system can further comprise the step of receiving data of the first dose of sex hormone administered to the individual.

In one aspect, described herein is a computer system for determining a need for adjustment of a dose of sex hormone, such as testosterone and/or estrogen, administered to an individual, comprising: one or more processors; and memory to store: one or more programs, the one or more programs comprising: instructions for: a) receiving data from determining the concentration of free sex hormone, such as testosterone and/or estrogen, in an individual receiving sex hormone therapy at a first dose, wherein the concentration of free sex hormone is determined by measuring i) a total SHBG concentration, ii) a total sex hormone concentration, such as testosterone and/or estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule, such as a testosterone and/or estrogen molecule, to the first monomer and an allosteric interaction between two binding sites of the SHBG dimer; d) sending a signal for providing a second dose of the sex hormone that is higher than the first dose when the free sex hormone concentration is below the lower end of the target therapeutic range; and e) sending a signal for providing a second dose of sex hormone that is lower than the first dose when the free sex hormone concentration is above the upper end of the target therapeutic range.

In one aspect, described herein is a non-transitory computer-readable storage medium storing one or more programs for determining a need for adjustment of a dose of sex hormone, such as testosterone and/or estrogen, administered to an individual, the one or more programs for execution by one or more processors of a computer system, the one or more programs comprising instructions for: a) receiving data from determining the concentration of free sex hormone, such as testosterone and/or estrogen, in an individual receiving sex hormone therapy at a first dose, wherein the concentration of free sex hormone is determined by measuring i) a total SHBG concentration, ii) a total sex hormone, such as testosterone and/or estrogen concentration, and iii) a total albumin concentration in a biological sample obtained from an individual, to determine free sex hormone concentration from the individual: b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for providing a second dose of sex hormone that is higher than the first dose when the free sex hormone concentration is below the lower end of the target therapeutic range; and e) sending a signal for providing a second dose of sex hormone that is lower than the first dose when the free sex hormone concentration is above the upper end of the target therapeutic range.

In one aspect, described herein is a method for determining a need for adjustment of a dose of sex hormone, such as testosterone and/or estrogen, administered to an individual comprising a) determining the concentration of free sex hormone, such as testosterone and/or free estrogen, in an individual receiving sex hormone therapy at a first dose, wherein the concentration of free sex hormone is determined by b) measuring i) a total SHBG concentration, ii) a total sex hormone, such as testosterone and/or estrogen, concentration, and iii) a total albumin concentration in a biological sample obtained from an individual, to determine free sex hormone concentration from the individual; c) attributing at least two distinct interconverting microstates of an unliganded SHBG dimer having a first monomer and a second monomer by applying the New Multi-Step Dynamic Binding Model with Complex Allostery to the data of steps a) and b); d) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; c) providing a second dose of sex hormone that is higher than the first dose when the free sex hormone concentration is below the lower end of the target therapeutic range; and f) providing a second dose of sex hormone that is lower than the first dose when the free sex hormone concentration is above the upper end of the target therapeutic range.

In some embodiments of any of the foregoing aspects, the step of attributing can be performed according to FIGS. 2, 3, 5, and 7. In some embodiments, of any of the foregoing aspects, the step of calculating can be performed according to FIG. 7 or Example 5. In some embodiments of any of the foregoing aspects, the individual is a male or female over the age of 35. In some embodiments of any of the foregoing aspects, the sex hormone disorder is selected from the group consisting of a testosterone deficiency, estrogen deficiency, an androgen deficiency, a hyperandrogenic disorder, an androgen expressing tumor, estrogen excess, estrogen expressing tumor, ovarian dysfunction, gynecomastia, menopause, and a hypogonadism disorder. In some embodiments of any of the foregoing aspects, the sex hormone disorder is a hyperandrogenic disorder selected from the group consisting of an acne disorder, a hirsutism disorder, and an androgenic alopecia disorder. In some embodiments of any of the foregoing aspects, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments of any of the foregoing aspects, the assay, method, system, or medium can further comprise the step of classifying the individual into categories based on additional clinical symptoms. In some embodiments of any of the foregoing aspects, the assay, method, system, or medium can further comprise the step of using the free sex hormone concentration, such as the testosterone and/or the free estrogen concentration, determined using the new Multistep Dynamic Binding Model with Complex Allostery to determine the dose or to individually adjust the dose of a formulation of sex hormone, such as testosterone and/or estrogen, for the treatment of a medical disease, taking into account patient's age, body weight and body mass index, medical conditions, including any co-morbid conditions, albumin and SHBG, and/or LH and FSH concentrations, and other patient-specific factors. In some embodiments of any of the foregoing aspects, instead of steps a-c, the data received is a previously calculated concentration of free sex hormone, such as free testosterone and/or free estrogen.

In one aspect, described herein is a method for determining a free sex steroid concentration in a biological sample comprising the following steps: identifying in the biological sample: i) a total SHBG concentration, ii) a total sex steroid concentration, and iii) an albumin concentration; b) attributing at least two distinct interconverting microstates of an unliganded SHBG dimer having a first monomer and a second monomer; c) calculating the free sex steroid concentration in the biological sample using an ensemble allostery model encompassing readjustment of a first equilibria between the microstates upon binding of a first sex steroid molecule to the first monomer and an allosteric interaction between two binding sites of the SHBG dimer. In some embodiments, the sex steroid is selected form the group consisting of a testosterone steroid, an estradiol steroid, and estrone steroid, and a dihydrotestosterone steroid.

In one aspect, described herein is a method diagnosing and treating a sex steroid disorder in a patient comprising the following steps: a) obtaining a biological sample from the patient; b) measuring in the biological sample obtained in step a): i. a total concentration of sex-hormone binding globulin (“SHBG”), which is a dimer having a first monomer and a second monomer, ii. a total concentration of the sex steroid, and iii. a concentration of albumin; c) determining the concentration of free sex steroid in the biological sample based on (i)-(iii) measured in step b), using an implementation of ensemble allostery model representing the binding equilibria (i) between the sex steroid and the SHBG dimer first monomer of the SHBG and between the sex steroid and the second monomer of the SHGB, wherein the unliganded SHBG has at least two distinct interconverting microstates, and wherein the first monomer and the second monomer have an allosteric interaction such that each of the microstates binds a first sex steroid molecule with a different affinity; and (ii) between the sex steroid and the albumin; d) diagnosing the patient with the sex steroid disorder when the free sex steroid concentration determined in step c) is below the lower limit of a normal free sex steroid concentration from a healthy individual; and e) administering an effective amount of sex steroid, sex steroid derivatives, and/or analogues thereof to the patient diagnosed in step d). In some embodiments, the sex steroid is selected form the group consisting of a testosterone steroid, an estradiol steroid, and estrone steroid, and a dihydrotestosterone steroid.

In some embodiments, the step of measuring in the biological sample further comprises measuring in the biological sample: iv. A concentration of at least a second sex steroid wherein the second sex steroid is different than the sex steroid that was measured in step (b). In other embodiments, the step of measuring in the biological sample further comprises measuring in the biological sample: iv. the concentration of one or more analytes. In other embodiments, the step of measuring in the biological sample further comprises measuring one or more of the concentrations in the biological sample using at least one analytical method. In other embodiments, the analytical method is selected from the group consisting of an immunoassay and a mass spectrometry-based assay. In some embodiments, the sex steroid disorder is selected from the group consisting of but not limited to testosterone deficiency, estrogen deficiency, an androgen deficiency, a hyperandrogenic disorder, an androgen expressing tumor, estrogen excess, estrogen expressing tumor, ovarian dysfunction, gynecomastia, menopause, and a hypogonadism disorder. In other embodiments, the sex steroid disorder comprises an estrogen deficiency or excess. In some embodiments, the treatment further comprises the step of: d) adjusting the dose of administered sex steroid, sex steroid derivatives, and/or analogues thereof for treatment of the sex steroid disorder. In other embodiments, the method further comprises the step of determining the concentration of the second sex steroid in the biological sample using an implementation of ensemble allostery model comprising readjusting a second equilibria between the microstates upon binding of the second sex steroid molecule to the first monomer and the allosteric interaction between two monomers of the SHBG.

In one aspect, described herein is a method of monitoring and optimizing treatment for a sex steroid deficiency in a patient comprising the following steps: a) obtaining a biological sample from the patient who is on a treatment of the sex steroid deficiency; b) measuring in the biological sample obtained in step a): i. a total concentration of sex-hormone binding globulin (“SHBG”), which is a dimer having a first monomer and a second monomer, ii. a total concentration of the sex steroid, and iii. a concentration of albumin; c) determining the concentration of free sex steroid in the biological sample based on (i)-(iii) measured in step b), using an implementation of ensemble allostery model representing the binding equilibria (i) between the sex steroid and the first monomer of the SHBG and between the sex steroid and the second monomer of the SHBG, wherein the unliganded SHBG has at least two distinct interconverting microstates and wherein the first monomer and the second monomer have an allosteric interaction such that each of the microstates binds the sex steroid molecule with a different affinity; and (ii) between the sex steroid and the albumin; d) identifying the patient as needing treatment optimization when the free sex steroid concentration determined in step c) is below the lower limit of a normal free sex steroid concentration from a healthy individual; and e) optimizing the treatment of the identified patient with the sex steroid disorder by administering a modified amount of the sex steroid, sex steroid derivatives, and/or analogues thereof to the identified patient. In some embodiments, the sex steroid is selected form the group consisting of a testosterone steroid, an estradiol steroid, and estrone steroid, and a dihydrotestosterone steroid.

In one aspect, described herein is a method of treating a sex steroid disorder in a patient comprising administering an effective amount of sex steroid, sex steroid derivatives, and/or analogues thereof to the patient wherein the patient's free sex steroid concentration is below the lower limit of a normal free sex steroid concentration from a healthy individual, wherein the patient's free sex steroid concentration is determined based on: i. total concentration of sex-hormone binding globulin (“SHBG”), which is a dimer having a first monomer and a second monomer, ii. total concentration of the sex steroid, and iii. concentration of albumin; and wherein the free sex steroid concentration is calculated by an implementation of ensemble allostery model representing the binding equilibria (i) between the sex steroid and the first monomer of the SHBG and between the sex steroid and the second monomer of the SHGB, wherein the unliganded SHBG has at least two distinct interconverting microstates, and wherein the first monomer and the second monomer have an allosteric interaction such that each of the microstates binds a first sex steroid molecule with a different affinity; and (ii) between the sex steroid and the albumin. In some embodiments, the sex steroid is selected form the group consisting of a testosterone steroid, an estradiol steroid, and estrone steroid, and a dihydrotestosterone steroid.

### BRIEF DESCRIPTION OF THE DRAWINGS

FIGS. 1A-1B demonstrate that free T values calculated using the Vermeulen/Sodergard/Mazer model—the commonly used method for determining free T concentrations—differ from those measured using equilibrium dialysis. FIG. 1A depicts a graph of free testosterone concentrations in samples derived from a randomized testosterone trial (The TED study) were measured using the equilibrium dialysis and plotted against those calculated using the Vermeulen/Sodergard/Mazer equation. Calculated free T concentrations differed systematically from the measured values. FIG. 1B depicts a Bland Altman plot revealing substantial discrepancy between the calculated and measured free T concentrations.

FIGS. 2A-2C demonstrate that binding of testosterone to SHBG displays complex behavior. FIG. 2A depicts a graph demonstrating that binding isotherms display complex behavior. Graded concentrations of testosterone were incubated overnight with various SHBG concentrations and the amount of bound testosterone was plotted against total T (added) concentrations (squares) 20 nM, (triangles) 10 nM, (circles) 5 nM SHBG. The fit curves represent the result of the linked fit of data to the new Multi-step Dynamic Binding Model with Complex Allostery (FIG. 3). FIG. 2B depicts a graph of the depletion of free testosterone by varying SHBG concentration which is best described by the new Multi-step Dynamic Binding Model with Complex Allostery. Shown is concentration of free testosterone in the equilibrium dialysis experiment. One side of the equilibrium dialysis membrane has varying concentration of SHBG in buffer, the other one has plain buffer. Constant concentration of testosterone is added to each well of multi-well dialyzer. (triangles) 8.7 nM. Curves are the result of the linked fit of data in Panel B to the new Multi-step Dynamic Binding Model with Complex Allostery (FIG. 3).

FIG. 2C depicts a graph of the heat of T:SHBG association measured by isothermal calorimetry (ITC). Presented is the integrated ITC curve with buffer heats subtracted. SHBG starting concentration is 5 μM. Experimental points are shown by squares, fit to the model in FIG. 3 is shown by a solid line.

FIG. 3 depicts a schematic representation of the new Multi-step Dynamic Binding Model with Complex Allostery of testosterone's binding to SHBG and albumin, developed in this study. Unliganded SHBG dimers (S₂, S₂′) exist in conformational equilibrium. Upon binding of the first testosterone molecule to microstates S₂ and S₂′ can result in conformationally heterogeneous intermediate states S₂T and S₂′T respectively. These singly-occupied microstates then converge to S₂T₂ upon binding of the second testosterone molecule.

FIGS. 4A-4B demonstrate a comparison of the Free Testosterone Concentrations Derived Using the Vermeulen Equation (23) as implemented by Mazer (24) or the New Algorithm Based on the new Multi-step Dynamic Binding Model with Complex Allostery with those measured using the equilibrium dialysis in samples from the Salpha reductase trial. FIG. 4A depicts a graph of a comparison of the free testosterone concentration calculated by the Vermculen equation and the new algorithm based on the new Multi-step Dynamic Binding Model with Complex Allostery to that measured by equilibrium dialysis in samples from a randomized testosterone trial in men. (▪) free testosterone concentrations derived using an algorithm based on new Multi-step Dynamic Binding Model with Complex Allostery; (●) free testosterone concentrations derived using the Vermeulen model (23) as implemented by Mazer (24). Solid lines are lines of best linear fit. Regression lines fit new Multi-step Dynamic Binding Model with Complex Allostery calculation (slope=1.01±0.01, regression line fitting the squares), and the Vermeulen model (slope 0.77±0.02, lower line fitting the dots). Magenta dashed line is the line of prefect correlation. FIG. 4B depicts Bland Altman plots of the relative frequency distribution of % difference of calculated and measured free testosterone using either the Vermeulen equation (squares) or the new algorithm based on the new Multi-step Dynamic Binding Model with Complex Allostery (black dots) The relative deviations from the measured value are distributed around 0 for new Multi-step Dynamic Binding Model with Complex Allostery model and are different from zero for the Vermeulen model. FIGS. 4C-4D depict a Comparison of the Free Testosterone Concentrations Derived Using the Vermeulen Equation or the New Algorithm Based on the new Multi-step Dynamic Binding Model with Complex Allostery with those measured using the equilibrium dialysis. FIGS. 4C-4D demonstrate a Comparison of the Free Testosterone Concentrations Derived Using the Vermeulen Equation or the New Algorithm Based on the new Multi-step Dynamic Binding Model with Complex Allostery with those measured using the equilibrium dialysis in samples from a randomized testosterone trial in men with ED. FIG. 4C depicts a graph of a comparison of the free testosterone concentration calculated by the Vermeulen equation and the new algorithm based on the new Multi-step Dynamic Binding Model with Complex Allostery to that measured by equilibrium dialysis in samples from a randomized testosterone trial in men. (squares) free testosterone concentrations derived using an algorithm based on new Multi-step Dynamic Binding Model with Complex Allostery; (circles) free testosterone concentrations derived using the Vermeulen model (23) as implemented by Mazer (24). Black regression line fits new Multi-step Dynamic Binding Model with Complex Allostery model calculation (slope=1.01±0.01 is shown) FIG. 4D depicts Bland Altman plots of the relative frequency of % difference of calculated and measured free testosterone using either the Vermeulen equation or the new algorithm based on the new Multi-step Dynamic Binding Model with Complex Allostery. The relative deviations from the measured value are distributed around 0 for the new Multi-step Dynamic Binding Model with Complex Allostery model and are different from zero for the Vermeulen model.

FIG. 5A depicts a graph of the binding isotherm. Graded concentrations of testosterone were incubated overnight with 20 nM SHBG and the amount of bound testosterone was plotted against total testosterone concentration. The fit curve represents the fit of data to the new Multi-step Dynamic Binding Model with Complex Allostery. FIG. 5B depicts a graph of the depletion curve. A constant concentration of testosterone (8.7 nM) was incubated with increasing SHBG concentrations. Free testosterone concentration is plotted against SHBG concentration. Solid line represents the fit of data to new Multi-step Dynamic Binding Model with Complex Allostery. The depletion of free testosterone by increasing SHBG concentrations is best described by the new Multi-step Dynamic Binding Model with Complex Allostery. FIG. 5C depicts a graph of the heat of testosterone and SHBG association measured by isothermal calorimetry. The integrated ITC curve was generated after subtracting the buffer heats. SHBG starting concentration is 5 μM. Experimental points are shown by (▪), and fit of the data to the new Multi-step Dynamic Binding Model with Complex Allostery model is shown by a solid line.

FIG. 6 depicts schematic representations of the various models tested in this study to examine SHBG:T interaction. Model A. Vermeulen's model, homogenous interaction of testosterone molecule with equal affinity for each monomer in SHBG dimer (Kd=1 nM); Model B, monomers within the SHBG dimer exhibit distinct affinity constants with Kd₁=1 nM and Kd₂ allowed to vary for data fits; Model C. Inter-subunit allostery with positive cooperativity for binding of two ligands such that Kd₂=nM, Kd₁ allowed to vary and Kd₂<Kd₁; Model D, allostery with negative cooperativity for binding of two ligands such that Kd₁=1 nM, Kd₂ allowed to vary and Kd₁<Kd₂; and, Model E. The new Multi-step Dynamic Binding Model with Complex Allostery which encompasses two distinct SHBG microstates in equilibrium such that the equilibria between the unliganded and mono-liganded states readjust as testosterone concentration is increased.

FIG. 7 depicts the thermodynamic Parameters associated with testosterone's binding to SHBG derived from the fit of binding isotherms and ITC data to new model. While this parameter set is not unique, together they consistently describe the binding isotherms, depletion curves and ITC data to the new Multi-step Dynamic Binding Model with Complex Allostery developed in this study. These were utilized to obtain FT values (cFT_{ZBJ}) in samples obtained in clinical trials.

FIGS. 8A-8B demonstrate that the binding of testosterone to SHBG displays complex allostery. FIG. 8A depicts a graph demonstrating that binding isotherms display significant non-linearity. Varying concentrations of testosterone were incubated with a fixed concentration of SHBG (5, 10 or 20 nM) and bound testosterone was plotted against total testosterone concentration. The binding isotherms were generated at 5, 10 and 20 nM SHBG. Curves represent the result of the fit of data to the new Multi-step Dynamic Binding Model with Complex Allostery. FIG. 8B depicts a graph demonstrating that depletion of FT by varying SHBG concentration is best described by the new Multi-step Dynamic Binding Model with Complex Allostery. Constant concentration of testosterone (6, 12, 17 or 32 nM) was incubated with increasing SHBG concentrations, and free testosterone concentration in buffer side was plotted against SHBG concentration. The curves are the result of the fit of data to the new Multi-step Dynamic Binding Model with Complex Allostery.

FIGS. 9A-9C depicts graphs depicting the fits of the various models of testosterone's binding to SHBG to the experimental data from binding isotherms, depletion experiments, and ITC. Left panels: The figures show the fits of data to the various models examined in this study. Right panels: The figures show corresponding residuals of the fit of data to various models of testosterone's binding to SHBG. Neither the Vermeulen's equation nor the simple allostery models adequately fit the experimental data from binding isotherms, depletion experiments, or ITC. The new Multi-step Dynamic Binding Model with Complex Allostery (model E) provided the optimal fit to the experimental data from all three methods.

FIG. 10 depicts a schematic of the control of testosterone levels.

FIG. 11 depicts a schematic of an exemplary system of determining free testosterone levels and/or dosages.

FIG. 12 depicts a device or a computer system 1000 comprising one or more processors 1300 and a memory 1500 storing one or more programs 1600 for execution by the one or more processors 1300.

FIGS. 13A-13E show that equilibrium dialysis experiments demonstrate that estradiol binding to SHBG dimer is a dynamic non-linear process. FIG. 13A displays the binding isotherm, generated by the titration of increasing SHBG concentrations in the presence of a fixed estradiol concentration (either 2.6 or 13.7 nM). The data from representative experiments conducted at the respective estradiol concentrations of either 2.6 nM (circles) or 13.7 nM (squares) are shown. The binding isotherm shows nonlinearity of binding as well as asymmetry around the 50% [BE2] point which is inconsistent with the prevalent notion of linear binding with a single dissociation constant, Kd. FIG. 13B depicts the corresponding depletion curve, generated by plotting the concentration of the free estradiol that was dialyzed into the buffer side of dialysis chamber against the SHBG concentration. FIG. 13C displays the fraction of estradiol that was bound to SHBG ([bound E2]/[total E2]) on the sample side of the dialysis chambers at each SHBG concentration. Each data point represents the average E2 and SHBG concentration from two experiments. FIG. 13D displays the fraction of estradiol that was free ([free E2]/[total E2]) at each SHBG concentration. Each data point represents the average of free E2 and SHBG concentration from two experiments. FIG. 13E is a plot of the apparent Kd vs the SHBG concentration in the equilibrium dialysis experiments performed at five E2 concentrations: 2.6 nM (circles), 3.8 nM (diamonds), 4.1 nM (triangles), 7.1 nM (inverted triangles), and 13.7 nM (squares). At each SHBG concentration, the apparent Kd was determined from the measured SHBG, and the measured free and bound estradiol concentrations. The apparent Kd varied non-linearly as the SHBG concentrations and SHBG:E2 ratios were varied.

FIGS. 14A-D show estradiol binding to SHBG alters the microenvironment of tryptophan residues and quenches the fluorescence emission intensity. FIG. 14A shows the relative distance of tryptophan residues from the estradiol ligand in the binding pocket. The residue coordinates were obtained from the crystal structure (PDB ID: 1LHU). The distances between the alpha carbons are mapped for the five tryptophan residues in SHBG to the C9 position on E2. FIG. 14B shows the steady state emission spectrum from tryptophan residues as the estradiol concentration was increased from subphysiologic (0.0001 nM) to supraphysiologic (100 nM) range. The data were collected with 1 mm slit width using 1ex of 290 nm and emission was collected from 310 nm to 410 nm. FIG. 14C is a plot of the changes in integrated emission from tryptophan residues in 20 nM SHBG at increasing estradiol concentration. The binding curve predicted by the extant, linear model of SHBG:E2 association assuming homogenous interaction with both monomer with a fixed Kd of 2 nM (solid curve) does not fit the experimental binding data, shown in the solid black symbols (FIG. 14C). FIG. 14D plots the residuals between the predicted (solid curve in FIG. 14C) and experimentally measured data points at graded E2 concentrations and shows that the prevailing linear binding model exhibits under and overestimation from the experimentally derived binding curve at various E2 concentrations.

FIGS. 15A-15C show trajectories of residues within the ligand binding pockets (LBP1 and LBP2 respectively) of the first and second monomer showing conformational coupling between the two SHBG monomers. The plots depict the temporal evolution of trajectories over 5 microsecond of the three structures: SHBG:0E2—unliganded SHBG dimer as shown in FIG. 15A; SHBG:1E2—singly-bound state in which only the first monomer is bound to estradiol as shown in FIG. 15B; and SHBG:2E2—doubly-bound state in which the second monomer also is occupied by estradiol as shown in FIG. 15C. Color with asterisk (*) represents monomer 1 (LBP1); color with plus sign (+) represents monomer 2 (LBP2). A comparison of the trajectories in FIG. 15A and FIG. 15B shows that the binding of estradiol to the LBP of the first monomer changes the population of conformational states not only in monomer 1 but also in monomer 2, providing evidence of the allosteric interaction between the monomers. FIG. 15C shows the subsequent binding of the second estradiol to the LBP of the second monomer in the doubly-bound state alters the conformational state of the LBP of the second monomer but also of the first previously occupied LBP of the first monomer, providing further evidence of the bidirectional inter-monomeric allostery.

FIGS. 16A-16E show time-resolved lifetime fluorescence spectroscopy using an extrinsic fluorescent probe, bis-ANS, demonstrates that estradiol binding significantly alters the global conformational state of the SHBG:E2 complex. FIG. 16A shows the raw data and fits to the phase delay and modulation ratio obtained at increasing estradiol concentrations titrated into a solution of 40 nM SHBG and 500 nM bis-ANS. The modulation frequencies were altered from 10 to 160 MHz to determine tphase and tmod at each SHBG:E2 ratio. The excited state lifetime data was satisfactorily fit to the most parsimonious model of 2 emitting species where the chi square values ranged from 0.8 to 2.1. FIGS. 16B-16C show the short and long lifetime components, respectively, as a function of estradiol concentrations. FIGS. 16D-16E show the change in fractional (f1 and f2) and fluorescence contribution from the bis-ANS populations exhibiting short and long singlet excited state lifetime components. Collectively, the data indicate that estradiol binding to SHBG induces global conformational change in the protein as evidenced by the significant change in relative populations of bis-ANS species exhibiting the long and short singlet excited state lifetimes.

FIGS. 17A-17C shows dynamic cross-correlation matrices for SHBG dimer illustrate that distant residues in the two monomers are conformationally coupled and respond to binding of estradiol to either of the two monomers. Inter-monomeric residue motion correlations were examined for the three states: unliganded (as shown in FIG. 17A; SHBG:0E2), Singly bound (as shown in FIG. 17B; SHBG:1E2) and doubly bound (as shown in FIG. 17C; SHBG:2E2). The quadrants depicting the inter-monomer correlation show changes in coordinated movement of the residues in the two monomers. Red color intensity stands for strength of residue correlation. The correlated motions whose absolute values were smaller than 0.3 were not included. The right panels show the location of distant residues at the inter-monomeric interface and ligand binding pockets in the two monomers which are conformationally coupled in the respective liganded states. Only the residues, which show correlated movement, are colored in monomer 1 (red) and monomer 2 (blue). Estradiol molecule is represented in purple color. Collectively, these data show that allosteric coupling in binding estradiol is manifested through coordinate, dynamic rearrangement of residues in each of the two SHBG monomers.

FIGS. 18A-18E show the two monomers within the SHBG dimer populate distinct conformational clusters, which change dynamically upon ligand binding. Relative frequency distribution of SHBG conformational states occupied by each of the monomers (as shown in FIG. 18A, Monomer 1; as shown in FIG. 18B monomer 2) in response to the binding of the first and the second estradiol molecule to SHBG dimer. The most parsimonious Markov State modeling analysis shows that six conformationally distinct states are involved in this dynamics as depicted in this histogram of the distribution of the two monomers in various conformational clusters in the unliganded, singly-bound and doubly-bound SHBG. Inset displays the color corresponding to the conformational clusters that each SHBG monomer occupies in the unbound and bound states. As shown in FIGS. 18A-18C, the monomers dynamically repartition predominantly in clusters 1-5 as the SHBG dimer transitions between unliganded, singly bound and doubly bound states. We posit that this multi-state conformational equilibria manifests as apparent Kd being sensitive to the SHBG/E2 ratio. For this to be true, one would expect that the conformational arrangement of residues in the ligand binding pockets in these conformational clusters would be distinct. There we generated pairwise overlays for the clusters occupied in the monomers in unbound, singly- and doubly-bound SHBG dimer. FIGS. 18C-18E show the spatial distinction in the orientation of ligand binding pocket residues in the various clusters. FIG. 18C shows the overlay of LBP residues in clusters 1 (marked with a letter A) and 2 (marked with a letter B). FIGS. 18D-18E illustrate the changes in LBP residues in clusters 3 (marked with a letter C), 4 (marked with a letter D) and 5 (marked with a letter E), which are predominantly occupied by monomers 1 and 2 in the singly bound states of SHBG dimer.

FIG. 19 shows estradiol binding to either of the two monomers alters the conformational energy landscape of the other monomer. The three panels collectively show the ensemble allosteric modulation of relative probabilities of population of clusters and respective transition rates between the conformational substrates in response to estradiol binding to monomers. The top section shows the two unliganded SHBG monomers populate distinct conformational states. The middle section shows upon estradiol binding to monomer 1, not only are the conformational states of monomer 1 impacted but the monomer 2 also is allosterically affected. The bottom section illustrates that estradiol binding to monomer 2 alters the conformational states of the already bound monomer 1 such that even in the fully bound state, the monomers within the SHBG dimer are not equivalent and exhibit conformational heterogeneity.

### DETAILED DESCRIPTION

The current model of sex hormone binding, including but not necessarily limited to testosterone and/or estrogen binding to SHBG assumes that each SHBG dimer binds two sex hormones, such as testosterone or estrogen molecules, respectively, and that each of the two binding sites on SHBG dimer has similar binding affinity. Equations to determine FT on the basis of this model were proposed by Vermeulen, Sodergard, Mazer and others. These linear equations proposed by Vermeulen, Sodergard, Mazer, and others are referred to as the prevailing equations/methods. Described herein is the characterization of sex hormone's binding to SHBG, such as testosterone's and/or estrogen's binding to SHBG, using (binding isotherms varying both ligand and protein) isothermal titration calorimetry. Based on the analyses of the data presented herein, described herein are methods, assays, and systems for, e.g., the diagnosis and treatment of sex hormone disorders that more accurately measure biological levels of sex hormone, such as testosterone and/or estrogen, than the prevailing equations/methods.

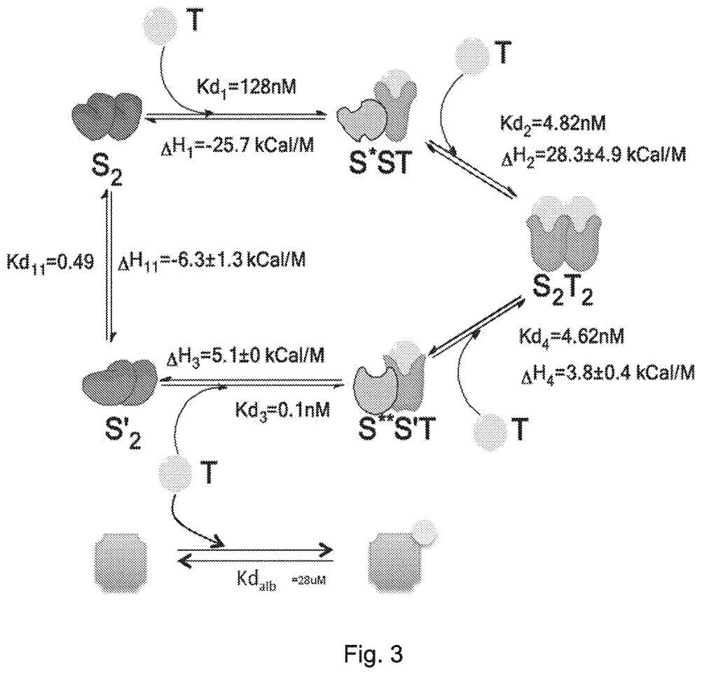

In one aspect, described herein is an assay comprising the steps of: a) measuring i) a total SHBG concentration, ii) a total sex hormone concentration, such as testosterone and/or estrogen concentration, and iii) a total albumin concentration in a biological sample obtained from an individual, to determine free sex hormone concentration, such as free testosterone or free estrogen concentration, from the individual; b) attributing at least two distinct interconverting microstates of an unliganded SHBG dimer having a first monomer and a second monomer by fitting the data of step a) to curves using the new Multi-step Dynamic Binding Model with Complex Allostery; c) calculating the free sex hormone concentration in the individual using the new Multi-step Dynamic Binding Model with Complex Allostery encompassing readjustment of a first equilibria between the microstates upon binding of a first sex hormone molecule to the first monomer and an allosteric interaction between two binding sites of the SHBG dimer. In some embodiments, step b is performed according to FIG. 3. In some embodiments, step c is performed according to Example 5.

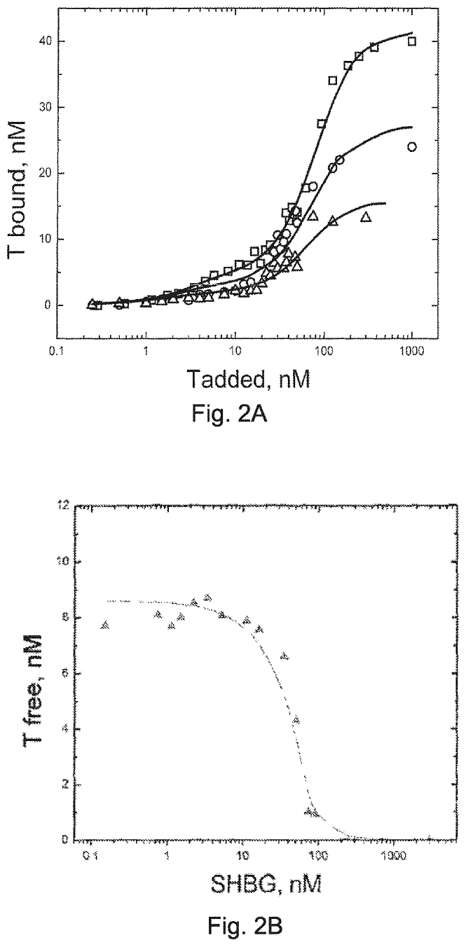

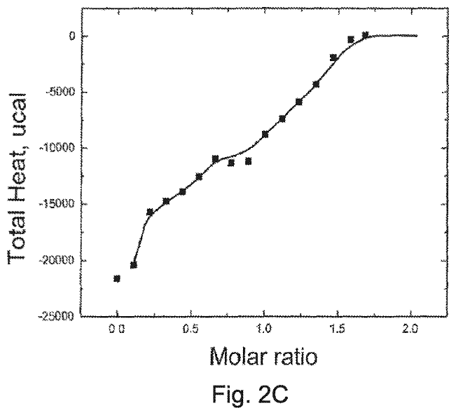

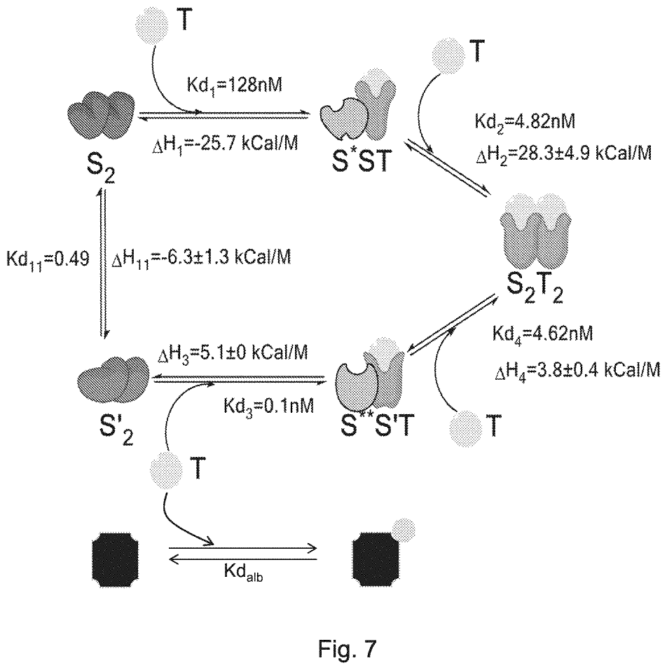

In one aspect, described herein is an assay comprising the steps of: a) measuring i) a total SHBG concentration, ii) a total sex hormone concentration, such as testosterone and/or estrogen concentration, and iii) a total albumin concentration in a biological sample obtained from an individual, to determine free sex hormone concentration, such as free testosterone and/or free estrogen concentration from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer. In some embodiments, step b) is performed according to FIGS. 2, 3, 5, and 7. In some embodiments, step c) is performed according to FIG. 7 and/or Example 5.

Formulas described herein relate to a model of sex hormone and SHBG binding, such as testosterone and/or estrogen and SHBG binding, in which two distinct interconverting microstates of an unliganded SHBG dimer having a first monomer and a second monomer. SHBG exists as one of two dimerized forms or microstates (see, e.g., FIG. 3), each of which can bind to a first sex hormone molecule with a different affinity. These two microstates of SHBG can interconvert. The new Multi-step Dynamic Binding Model with Complex Allostery described herein is a model of the interaction between the sex hormone and SHBG which accounts for both a first equilibria between the microstates upon binding of a first sex hormone molecule to the first monomer and an allosteric interaction between two binding sites of the SHBG dimer.

Without wishing to be bound by theory, sex hormones, such as testosterone and estrogen, are commonly known to persons of ordinary skill in the art. For example, they can be shown by compound with the formulae:

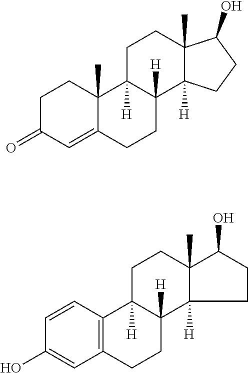

Testosterone is also known under the chemical name 17-β-hydroxyandrost-4-en-3-one (or 4 androsten 17β-ol-3-one) which can be obtained in various ways: it may be isolated and purified from nature or synthetically produced by any manner. Testosterone is the major androgen in males and is controlled by luteinizing hormone (LH). LH is released from the anterior pituitary exerting the primary control on testosterone production, and acting directly on the Leydig cells in the testes, where testosterone is produced. Testosterone stimulates adult maturation of external genitalia and secondary sex organs, and the growth of beard, auxiliary and pubic hair. In addition, testosterone has anabolic effects leading to increased linear growth, nitrogen retention, and muscular development. Clinical evaluation of serum testosterone, along with serum LH, assists in evaluation of hypogonadal males. Major causes of lowered testosterone in males include hypogonadotropic hypogonadism, testicular failure, hyperprolactinemia, hypopituitarism, some types of liver and kidney diseases, and critical illness. Estrogen is the main female hormone produced by ovaries in women. In both men and women, it can be converted from testosterone or produced by adrenals. Estrogen excess or deficiency is implicated is several disorders and clinical conditions including but not limited to pubertal disorders, menopause, ovarian and/or adrenal tumors, breast/ovarian tumors, infertility, gynecomastia, bone health, sexual function, and amenorrhea.

Testosterone levels are much lower in females compared to males. Estrogen levels in men are much lower than females. The major sources of testosterone in females are the ovaries, the adrenal glands, and the peripheral conversion of precursors, specifically the conversion of androstenedione to testosterone. In females, the normal levels of androgens may provide a substrate for estrogen production. Increased serum testosterone levels in females may be indicative of polycystic ovary syndrome and adrenal hyperplasia, among other conditions. Increased estrogen levels in men can cause gynecomastia.

Sex hormones, such as-testosterone and estrogen, bind strongly to sex hormone-binding globulin (SHBG). As used herein. “SHBG” refers to a glycoprotein that binds to sex hormones, i.e. androgens and estrogens. Sequences for SHBG of a number of species are known in the art, e.g. human SHBG (NCBI Gene ID: 6462) mRNA (NCBI Seq Ref: NM_001040) and polypeptide (NCBI Seq Ref: NP_001031).

Sex hormones, such as testosterone and estrogen, bind with lower affinity to albumin. As used herein, “albumin” refers to an unglycosylated protein, e.g. serum albumin. Sequences for albumin of a number of species are known in the art, e.g. human serum albumin (NCBI Gene ID: 213) mRNA (NCBI Seq Ref: NM_000477) and polypeptide (NCBI Seq Ref: NP_00468).

In some embodiments, the concentration of SHBG, sex hormone (such as testosterone or estrogen) and/or albumin can be measured by an assay selected from the group consisting of an immunoassay, a binding assay, and a mass-spectrometry assay. In some embodiments, determining the level of SHBG or albumin polypeptide can comprise the use of a method selected from the group consisting of: enzyme linked immunosorbent assay; chemiluminescent immunosorbent assay; electrochemiluminescent immunosorbent assay; fluorescent immunosorbent assay; dye linked immunosorbent assay; immunoturbidimetric assay; immunonephelometric assay; dye-based photometric assay; western blot; immunoprecipitation; radioimmunological assay (RIA); radioimmunometric assay; immunofluorescence assay and mass spectroscopy. In some embodiments, determining the level of total sex hormone, such as total testosterone or estrogen, can comprise the use of a method selected from the group consisting of: bioassays, radioligand assays, radioimmunoassay; liquid chromatography tandem mass spectroscopy; enzyme linked immunosorbent assay; chemiluminescent immunosorbent assay; electrochemiluminescent immunosorbent assay; fluorescent immunosorbent assay; and high-pressure liquid chromatography.

Total sex hormone levels, such as total testosterone or total estrogen levels in a subject can be measured by ordinary methods commonly known in the art. For example, numerous assays for testosterone are known to those of skill in the art. See, e.g., Marcus and Durnford, Steroids 46: 975-86 (1985); Giraudi et al., Steroids 52: 423-4 (1988); Ooi and Donnelly, Clin. Chem. 44: 2178-82 (1988); Dorgan et al., Steroids 67: 151-8 (2002); Choi et al., Clin. Chem. 49: 322-5 (2003). Additionally, U.S. Patent Application 2008/0166697, which is incorporated herein in its entirety by reference that discloses methods to measure testosterone levels by Mass spectrometry. The measurement of total sex hormone levels, such as total testosterone or total estrogen levels, can be achieved by double isotope techniques and is commonly used for elucidation of difficult clinical diagnoses such as male or female pseudohermaphroditism, congenital adrenal hyperplasia and the androgen insensitivity syndrome. Commercially available kits can readily be used to measure sex hormone, such as testosterone and estrogen, such as commercially available kits from Pantex, DSL, Incostar and the like. Alternatively, serum sex hormone levels, such as testosterone or estrogen levels, can be measured by isotope dilution-liquid chromatography (see Bui et al., Ann Clin Biochem 2010; 47:248-252), in the blood, urine or saliva, (commercially available testosterone and estrogen assay from Salimetrics, LLC, State College, Pa.). The normal range for sex hormone levels, such as testosterone and/or estrogen levels in men and women is broad and varies by stage of maturity and age.

Kits for measuring total sex hormone levels, such as testosterone or estrogen levels, are commercially available, e.g., from Progene (Cat No. 991547; Cincinnati, Ohio); ZRT (Cat No. 84403; Beaverton, Oreg.); Cayman Chemicals (Cat No. 582701; Ann Arbor, Mich.); and MP Biomedicals (Cat No. 07BC-1115; Orangeburg, N.Y.), and others.

Kits for measuring SHBG levels are commercially available, e.g., from Genway Biotech Inc. (Cat No. GWB-D8BSDE; San Diego, Calif.); DRG (Cat No EIA-2996; Springfield, N.J.; and R&D Systems (Cat No. DSHBG0; Minneapolis, Minn.) and from others.

In some embodiments, e.g., albumin levels can be determined by dye-based photometric assays on an automated analyzer. Dye-based photometric assays are commercially available (e.g. the Albumin FS™ kits; DiaSys Diagnostic Systems Gmb; Holzheim, Germany or the Albumin reagent, Cat # OSR6102; Beckman Coulter; Brea, Calif.). Automated analyzers are commercially available (e.g. the AU2700 or AU5400 from Beckman Coulter, Brea, Calif.). Systems which are designed specifically for the determination of serum albumin levels are also available commercially (e.g. the Careside Analyzer™, Careside Inc., Culver City, Calif.). In some embodiments, the level of albumin levels can be determined using immunoassays, e.g. the Human Serum Albumin ELISA Kit (Cat #110; Alpha Diagnostic International; San Antonio, Tex.).

In some embodiments, the assays, methods and/or systems described herein comprise a step of transforming SHBG, albumin, and/or sex hormone (such as testosterone and/or estrogen) into a detectable composition and measuring the level of the detectable composition. As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a polypeptide can be bound by an antibody reagent.

Transformation, measurement, and/or detection of a target molecule, e.g. a sex hormone molecule, such as a testosterone or estrogen molecule, or SHBG polypeptide can comprise contacting a sample obtained from a subject with a reagent (e.g. a detection reagent) which is specific for the target, e.g., a SHBG-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure polypeptides are well known to a skilled artisan. Such methods include, e.g., ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for SHBG are commercially available and can be used for the purposes of the invention to measure protein expression levels, e.g. anti-SHBG (Cat. No. ab31401; Abcam, Cambridge Mass.). Alternatively, since the amino acid sequences for SHBG are known and publicly available at NCBI website, one of skill in the art can raise their own antibodies against these polypeptides of interest for the purpose of the invention.

In some embodiments, the measurement of, e.g., SHBG, albumin, sex hormone (such as estrogen or testosterone) comprises immunochemistry. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays. e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein peptide complex. The complex is then detected by a variety of methods known in the art. An immunoassay also often involves the use of a detection antibody.

Enzyme-linked immunosorbent assay, also called ELISA, enzyme immunoassay or ETA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., SHBG, or sex hormone, such as estrogen or testosterone, as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Ocllerich, M. 1984, J. Clin. Chem. Clin. Biochem. 22:895-904. These references are hereby incorporated by reference in their entirety.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In addition, polypeptide levels can be measured using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000). In certain embodiments, a gas phase ion spectrophotometer is used.

In other embodiments, laser-desorption/ionization mass spectrometry is used to analyze the level of a protein. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing Philadelphia, 1985; and Kirk-Othmer Encyclopedia of Chemical Technology, 4.sup.th ed. Vol. 15 (John Wiley & Sons, New York 1995), pp. 1071-1094. The mass spectrometers and their techniques are well known to those of skill in the art.

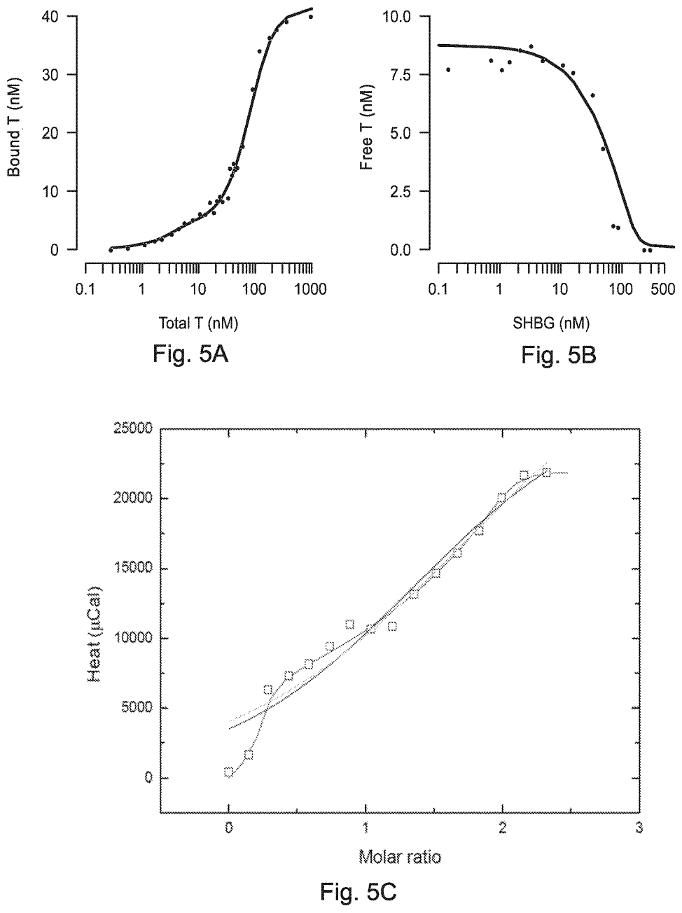

In some non-limiting embodiments of the various aspects described herein, it is determined whether a subject has a normal level of free testosterone. In other non-limiting embodiments of the various aspects described herein, it is determined whether a subject has a normal level of free estrogen. In some embodiments, a normal free testosterone level can be a level greater than about 100 pg/mL (e.g. as measured using the assays of FIGS. 5A-5C and Example 5). In some embodiments, a normal free testosterone level can be a level greater than about 105 pg/mL (e.g. as measured using the assays of FIGS. 5A-5C and Example 5). In some embodiments, a normal free testosterone level can be a level greater than about 108 pg/mL (e.g. as measured using the assays of FIGS. 5A-5C and Example 5). In some embodiments, a normal free testosterone level can be a level greater than about 110 pg/mL (e.g. as measured using the assays of FIGS. 5A-5C and Example 5). In some embodiments, a normal free testosterone level can be a level greater than about 112 pg/mL (e.g. as measured using the assays of FIGS. 5A-5C and Example 5). In some embodiments, a normal free testosterone level can be a level greater than about 114 pg/mL (e.g. as measured using the assays of FIGS. 5A-5C and Example 5). In some embodiments, a normal free testosterone level can be a level greater than about 114.6 pg/mL (e.g. as measured using the assays of FIGS. 5A-5C and Example 5). In some embodiments, a normal free testosterone level can be a level greater than about 116 pg/mL (e.g. as measured using the assays of FIGS. 5A-5C and Example 5). In some embodiments, a normal free testosterone level can be a level greater than about 118 pg/mL (e.g. as measured using the assays of FIGS. 5A-5C and Example 5). In some embodiments, a normal free testosterone level can be a level greater than about 120 pg/mL (e.g. as measured using the assays of FIGS. 5A-5C and Example 5). In some embodiments, a normal free testosterone level can be a level greater than about 122 pg/mL (e.g. as measured using the assays of FIGS. 5A-5C and Example 5).

In some non-limiting embodiments of the various aspects described herein, it is determined whether a subject has a free testosterone level within the healthy target range of free testosterone. In other non-limiting embodiments of the various aspects described herein, it is determined whether a subject has a free estrogen level within the healthy target range of free estrogen. In some embodiments, the target range of free testosterone can be a level from about 120 pg/mL to about 375 pg/mL (e.g. as measured using the assays of FIGS. 5A-5C and Example 5). In some embodiments, the target range of free testosterone can be a level from about 140 pg/mL to about 350 pg/mL (e.g. as measured using the assays of FIGS. 5A-5C and Example 5). In some embodiments, the target range of free testosterone can be a level from about 150 pg/mL to about 340 pg/mL (e.g. as measured using the assays of FIGS. 5A-5C and Example 5). In some embodiments, the target range of free testosterone can be a level from about 160 pg/mL to about 320 pg/mL (e.g. as measured using the assays of FIGS. 5A-5C and Example 5). In some embodiments, the target range of free testosterone can be a level from about 164 pg/mL to about 314 pg/mL (e.g. as measured using the assays of FIGS. 5A-5C and Example 5).

In one aspect, described herein is a method for treating an individual suspected of having an sex hormone disorder, such as an androgen disorder, the method comprising: a) prompting administering a pharmaceutically effective amount of testosterone to an individual who has had a free testosterone level determined by measuring a) a total SHBG concentration, b) a total testosterone concentration, and c) a total albumin concentration in a biological sample from the individual suspected of having an androgen disorder, b) receiving data comprising measuring i) a total SHBG concentration, ii) a total testosterone concentration, and iii) a total albumin concentration in a biological sample obtained from an individual, to determine free testosterone concentration from the individual; c) at least two distinct interconverting microstates of an unliganded SHBG dimer having a first monomer and a second monomer by fitting the data of step a) to curves using the new Multi-step Dynamic Binding Model with Complex Allostery; d) and calculating the free testosterone concentration in the individual using the new Multi-step Dynamic Binding Model with Complex Allostery encompassing readjustment of a first equilibria between the microstates upon binding of a first testosterone molecule to the first monomer and an allosteric interaction between two binding sites of the SHBG dimer and; e) administering to an individual having a free testosterone concentration below 114.6 pg/mL and f) not administering to an individual having a free testosterone concentration above 114.6 pg/mL. In some embodiments, step c) is performed according to FIG. 3. In some embodiments, step d) is performed according to Example 5. In some embodiments, the method can further comprise the step of classifying the individual into categories based on additional clinical symptoms.

In one aspect, described herein is a method for treating an individual suspected of having a sex hormone disorder such as an estrogen disorder, the method comprising: a) prompting administering a pharmaceutically effective amount of estrogen to an individual who has had a free estrogen level determined by measuring a) a total SHBG concentration, b) a total estrogen concentration, and c) a total albumin concentration in a biological sample from the individual suspected of having an estrogen disorder, b) receiving data comprising measuring i) a total SHBG concentration, ii) a total estrogen concentration, and iii) a total albumin concentration in a biological sample obtained from an individual, to determine free estrogen concentration from the individual; c) at least two distinct interconverting microstates of an unliganded SHBG dimer having a first monomer and a second monomer by fitting the data of step a) to curves using the new Multi-step Dynamic Binding Model with Complex Allostery; d) and calculating the free estrogen concentration in the individual using the new Multi-step Dynamic Binding Model with Complex Allostery encompassing readjustment of a first equilibria between the microstates upon binding of a first estrogen molecule to the first monomer and an allosteric interaction between two binding sites of the SHBG dimer and; e) administering to an individual having a free estrogen concentration below a particular concentration and f) not administering to an individual having a free estrogen concentration above a particular concentration. In some embodiments, the method can further comprise the step of classifying the individual into categories based on additional clinical symptoms.

In one aspect, described herein is a method for treating an individual suspected of having a sex hormone disorder, such as an androgen disorder, comprising: a) administering a pharmaceutically effective amount of testosterone to an individual who has had a free testosterone level determined by measuring i) a total SHBG concentration, ii) a total testosterone concentration, and iii) a total albumin concentration in a biological sample from the individual suspected of having an androgen disorder; b) attributing at least two distinct interconverting microstates of an unliganded SHBG dimer having a first monomer and a second monomer by applying the New Multi-Step Dynamic Binding Model with Complex Allostery to the data of step a); c) calculating the free testosterone concentration in the individual using an the New Multi-step Dynamic Binding Model with Complex Allostery encompassing readjustment of a first equilibria between the microstates upon binding of a first testosterone molecule to the first monomer and an allosteric interaction between two binding sites of the SHBG dimer; d) administering a pharmaceutically effective amount of testosterone to an individual having a free testosterone concentration below the lower limit of a normal free testosterone concentration from a healthy individual (e.g., in one embodiment, below 114.6 pg/ml); and e) not administering a pharmaceutically effective amount of testosterone to an individual having a free testosterone concentration above the lower limit of the normal free testosterone concentration from a healthy individual (e.g., in one embodiment, above 114.6 pg/ml, the lower limit of this assay). One of skill in the art understands that the lower limit of a normal free testosterone concentration will vary according to assay used to detect total testosterone, SHBG, and albumin and will vary in a different patient populations. Thus the lower limit used for implementation should correspond to the lower limit obtained in a healthy individual using the same assays as used on the biological sample from the test individual, and the lower limit used should be the lower limit from a healthy individual of the same age and type of the test individual. Thus, the computer systems and media will result in sending a signal for administering to an individual having a free testosterone concentration below the lower limit of the normal in that assay, or sending a signal for not administering to an individual having a free testosterone concentration above the lower limit of that assay. In some embodiments, step c) is performed according to FIGS. 2, 3, 5, and 7. In some embodiments, step d) is performed according to FIG. 7 or Example 5.

In one aspect, described herein is a method for treating an individual suspected of having a sex hormone disorder, such as an estrogen disorder, comprising: a) administering a pharmaceutically effective amount of estrogen disorder to an individual who has had a free estrogen level determined by measuring i) a total SHBG concentration, ii) a total estrogen concentration, and iii) a total albumin concentration in a biological sample from the individual suspected of having an estrogen disorder; b) attributing at least two distinct interconverting microstates of an unliganded SHBG dimer having a first monomer and a second monomer by applying the New Multi-Step Dynamic Binding Model with Complex Allostery to the data of step a); c) calculating the free estrogen concentration in the individual using an the New Multi-step Dynamic Binding Model with Complex Allostery encompassing readjustment of a first equilibria between the microstates upon binding of a first estrogen molecule to the first monomer and an allosteric interaction between two binding sites of the SHBG dimer; d) administering a pharmaceutically effective amount of estrogen to an individual having a free estrogen concentration below the lower limit of a normal free estrogen concentration from a healthy individual; and e) not administering a pharmaceutically effective amount of estrogen to an individual having a free estrogen concentration above the lower limit of the normal free estrogen concentration from a healthy individual. One of skill in the art understands that the lower limit of a normal free estrogen concentration will vary according to assay used to detect total estrogen, SHBG, and albumin and will vary in a different patient populations. Thus the lower limit used for implementation should correspond to the lower limit obtained in a healthy individual using the same assays as used on the biological sample from the test individual, and the lower limit used should be the lower limit from a healthy individual of the same age and type of the test individual. Thus, the computer systems and media will result in sending a signal for administering to an individual having a free estrogen concentration below the lower limit of the normal in that assay, or sending a signal for not administering to an individual having a free estrogen concentration above the lower limit of that assay.

As used herein, “androgen disorder” refers to a condition arising from and/or characterized by abnormal levels of one or more androgens, e.g. a condition arising from and/or characterized by abnormally low levels of testosterone. In some embodiments, the androgen disorder is selected from the group consisting of a testosterone deficiency, an androgen deficiency, a hyperandrogenic disorder, and a hypogonadism disorder. In some embodiments, the androgen disorder is a hyperandrogenic disorder selected form the group consisting of a polycystic ovary syndrome, an acne disorder, a hirsutism disorder, an androgen-expressing tumor, and an androgenic alopecia disorder.

As used herein, “estrogen disorder” refers to a condition arising from and/or characterized by abnormal levels of one or more estrogens, e.g. a condition arising from and/or characterized by abnormally low levels of estradiol. In some embodiments, the estrogen disorder is selected from the group consisting of a estrogen deficiency, an estrogen excess, a hyperestrogenic disorder, and a hypoestrogenic disorder. In some embodiments, the estrogen disorder is a ovarian dysfunction disorder, gynecomastia, estrogen expressing tumor, pubertal disorder or aromatase gene disorder.

In some embodiments, the methods described herein relate to treating a subject having, suspected as having, or diagnosed as having an androgen disorder, e.g. with low testosterone levels. Subjects having low testosterone can be identified by a physician using the methods described herein, alternatively, combined with current methods of diagnosing low testosterone. Symptoms and/or complications of low testosterone which characterize these conditions and aid in diagnosis are well known in the art and include but are not limited to, decreased sexual desire (e.g., decreased libido), erectile dysfunction (ED), decreased concentration, amenorrhea, loss of muscle mass, breast enlargement, fatigue, low mood, and the like.

In some embodiments, the methods described herein relate to treating a subject having, suspected as having, or diagnosed as having an estrogen disorder. Subjects having abnormal levels of estrogen can be identified by a physician using the methods described herein, alternatively, combined with current methods of diagnosing abnormal estrogen levels. Symptoms and/or complications of low estrogen which characterize these conditions and aid in diagnosis are well known in the art and include but are not limited to, decreased sexual desire (e.g., decreased libido), infertility, subfertility, over or under production by ovaries.

In one aspect, described herein is a method for determining a need for adjustment of a dose of testosterone administered to an individual comprising: a) determining the concentration of free testosterone in an individual receiving testosterone therapy at a first dose, wherein the concentration of free testosterone is determined by; b) receiving data comprising measuring i) a total SHBG concentration, ii) a total testosterone concentration, and iii) a total albumin concentration in a biological sample obtained from an individual, to determine free testosterone concentration from the individual; c) attributing at least two distinct interconverting microstates of an unliganded SHBG dimer having a first monomer and a second monomer by fitting the data of step a) to curves using the new Multi-step Dynamic Binding Model with Complex Allostery; d) and calculating the free testosterone concentration in the individual using the new Multi-step Dynamic Binding Model with Complex Allostery encompassing readjustment of a first equilibria between the microstates upon binding of a first testosterone molecule to the first monomer and an allosteric interaction between two binding sites of the SHBG dimer and; e) providing a second dose of testosterone that is higher than the first dose when the free testosterone concentration is below 164 pg/mL; and f) providing a second dose of testosterone that is lower than the first dose when the free testosterone concentration is above 314 pg/mL. In some embodiments, step c) is performed according to FIG. 3. In some embodiments, step d) is performed according to Example 5. In some embodiments, the method can further comprise the step of classifying the individual into categories based on additional clinical symptoms.

In one aspect, described herein is a method for determining a need for adjustment of a dose of estrogen administered to an individual comprising: a) determining the concentration of free estrogen in an individual receiving estrogen therapy at a first dose, wherein the concentration of free estrogen is determined by; b) receiving data comprising measuring i) a total SHBG concentration, ii) a total estrogen concentration, and iii) a total albumin concentration in a biological sample obtained from an individual, to determine free estrogen concentration from the individual; c) attributing at least two distinct interconverting microstates of an unliganded SHBG dimer having a first monomer and a second monomer by fitting the data of step a) to curves using the new Multi-step Dynamic Binding Model with Complex Allostery; d) and calculating the free estrogen concentration in the individual using the new Multi-step Dynamic Binding Model with Complex Allostery encompassing readjustment of a first equilibria between the microstates upon binding of a first estrogen molecule to the first monomer and an allosteric interaction between two binding sites of the SHBG dimer and; e) providing a second dose of estrogen that is higher than the first dose when the free estrogen concentration is below a particular concentration; and f) providing a second dose of estrogen that is lower than the first dose when the free estrogen concentration is above a particular concentration. In some embodiments, the method can further comprise the step of classifying the individual into categories based on additional clinical symptoms.

In one aspect, described herein is a method for determining a need for adjustment of a dose of testosterone administered to an individual comprising a) determining the concentration of free testosterone in an individual receiving testosterone therapy at a first dose, wherein the concentration of free testosterone is determined by b) measuring i) a total SHBG concentration, ii) a total testosterone concentration, and iii) a total albumin concentration in a biological sample obtained from an individual, to determine free testosterone concentration from the individual; c) attributing at least two distinct interconverting microstates of an unliganded SHBG dimer having a first monomer and a second monomer by applying the New Multi-Step Dynamic Binding Model with Complex Allostery to the data of steps a) and b); d) calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer; e) providing a second dose of testosterone that is higher than the first dose when the free testosterone concentration is below the lower end of the target therapeutic range (e.g. 164 pg/ml); and f) providing a second dose of testosterone that is lower than the first dose when the free testosterone concentration is above the upper end of the target therapeutic range (314 pg/ml). In another embodiment, the target therapeutic range could vary with the age of the patient, co-morbid conditions, and the types of assays used for measuring total testosterone, SHBG and albumin, in which case sending the signal for providing a second dose of testosterone that is higher than the first dose when the free testosterone concentration is below the lower end of the target therapeutic range for that age, co-morbid conditions and assay. In some embodiments, step c) is performed according to FIGS. 2, 3, 5, and 7. In some embodiments, step d) is performed according to FIG. 7 or Example 5.

In one aspect, described herein is a method for determining a need for adjustment of a dose of estrogen administered to an individual comprising a) determining the concentration of free estrogen in an individual receiving estrogen therapy at a first dose, wherein the concentration of free estrogen is determined by b) measuring i) a total SHBG concentration, ii) a total estrogen concentration, and iii) a total albumin concentration in a biological sample obtained from an individual, to determine free estrogen concentration from the individual; c) attributing at least two distinct interconverting microstates of an unliganded SHBG dimer having a first monomer and a second monomer by applying the New Multi-Step Dynamic Binding Model with Complex Allostery to the data of steps a) and b); d) calculating the free estrogen concentration in the individual using the New Multi-step Dynamic Binding Model with Complex Allostery encompassing readjustment of a first equilibria between the microstates upon binding of a first estrogen molecule to the first monomer and an allosteric interaction between two binding sites of the SHBG dimer; e) providing a second dose of estrogen that is higher than the first dose when the free estrogen concentration is below the lower end of the target therapeutic range; and f) providing a second dose of estrogen that is lower than the first dose when the free estrogen concentration is above the upper end of the target therapeutic range. In another embodiment, the target therapeutic range could vary with the age of the patient, co-morbid conditions, and the types of assays used for measuring total estrogen, SHBG and albumin, in which case sending the signal for providing a second dose of estrogen that is higher than the first dose when the free estrogen concentration is below the lower end of the target therapeutic range for that age, co-morbid conditions and assay.

In some embodiments, the assay further comprises the step of determining the concentration of at least one steroid other than the steroid being measured. As used herein, the term “steroid” refers to a chemical substance comprising three cyclohexane rings and a cyclopentane ring. In some embodiments, the steroid other than the steroid being measured is selected from the group consisting of testosterone, an estradiol steroid, an estrone steroid, and a dihydrotestosterone steroid.

In some embodiments, the individual of the methods and systems described herein is an individual who has or has been diagnosed with a disease selected from the group consisting of: diabetes, human immunodeficiency virus (HIV), hepatitis B, hepatitis C, androgen insensitivity, acromegaly, anorexia, muscular dystrophy, liver disease, cancer, cachexia, malnutrition, nephrotic syndrome and obesity, and other conditions in which SHBG and albumin concentrations are altered.

In some embodiments, the individual of the methods and systems described herein is a male. In some embodiments, the individual of the methods and systems described herein is a male over the age of 35, e.g. over the age of 40, over the age of 45, over the age of 50, over the age of 55, or over the age of 60.

In some embodiments, the individual of the methods and systems described herein is a female. In some embodiments, the individual of the methods and systems described herein is a female over the age of 35, e.g. over the age of 40, over the age of 45, over the age of 50, over the age of 55, or over the age of 60.

As used herein, “testosterone therapy” refers to the administration of testosterone or an analogue thereof, e.g. to treating an androgen disorder. The term “or analogue thereof” includes any useful metabolite or precursor of testosterone, for example the metabolite dihydrotestosterone. In some embodiments, a testosterone analogue can be, e.g., a testosterone ester such as testosterone cypionate, enanthate or propionate or a combination thereof, prodrug or fatty acid ester of testosterone; a fatty acid ester of testosterone of long chain (i.e., 14 or more carbons); methyltestosterone (in which the methyl group is covalently bonded to the testosterone nucleus as the C17 position to inhibit hepatic metabolism); a testosterone alkyl ester; an undecanoate acid ester of testosterone; testosterone undecanote; or a composition as disclosed, e.g. in US Patent Application US2011/0251167 which is incorporated herein in its entirety by reference.

As used herein, “estrogen therapy” refers to the administration of estrogen or an analogue thereof, e.g. to treating an estrogen disorder. The term “or analogue thereof” includes any useful metabolite or precursor of estrogen. In some embodiments, a estrogen analogue can be, e.g., a estrogen ester.

Numerous compositions are available for testosterone or estrogen therapies, e.g. patches or injections; intramuscular injections, implants, oral formulation of combined estrogens, oral tablets of alkylated testosterone (e.g., methyltestosterone), transdermal formulations such as the topical gels and solutions, or topical patches, and the like.

The formulation of the testosterone or testosterone derivatives and analogues or salts thereof should provide a dose of testosterone adequate to maintain the male subject's serum total testosterone level within the normal male range (approximately 300 to 1000 ng/dL range), based on measures of serum total testosterone. The formulation of the estrogen or estrogen derivatives and analogues or salts thereof should provide a dose of estrogen adequate to maintain the female subject's serum total estrogen level within the normal female range based on measures of serum total estrogen. The pharmaceutically effective amount of the testosterone, estrogen, testosterone derivatives and analogues or salts, or estrogen derivatives and analogues or salts thereof present in the compositions as disclosed herein depends on the patient's starting serum total testosterone or estrogen and the mode of administration. For oral administration, the compositions are preferably provided in the form of tablets containing 0.01, 0.05, 0.1, 0.5, 1.0, 2.5, 5.0, 10.0, 15.0, 25.0, 50.0 and 100 milligrams of active ingredient for the symptomatic adjustment of the dosage to the subject to be treated. An effective amount of the drug is ordinarily supplied at a dosage level of from about 0.0002 mg/kg to about 50 mg/kg of body weight per day. The range is more particularly from about 0.001 to 7 mg/kg of body weight per day. For example, testosterone and testosterone derivatives and analogues or salts thereof delivered by intramuscular injections may be provided in injections of 50 to 750 mg every one to 4 weeks. In one embodiment, testosterone and testosterone derivatives and analogues or salts thereof are provided by intramuscular injections of 100 to 500 mg every 1 to 4 weeks. In one class of this embodiment, testosterone and testosterone derivatives and analogues or salts thereof are provided by intramuscular injections of 50 to 250 mg every 1 to 4 weeks. In some embodiments, the total estrogen levels in men is in the range of 10 to 60 pg/mL. In some embodiments the total estrogen levels in women in range of 10-3000 pg/mL depending on age, menopause and/or pregnancy status.

Sex hormones, such as testosterone and estrogen, and derivatives and analogues or salts thereof may be provided in gel or cream forms in doses of 20 to 200 mg per day. In one embodiment, testosterone and testosterone derivatives and analogues or salts thereof are provided in a gel at doses of 50 to 100 mg/day, particularly 50 mg/day, 75 mg/day and 100 mg/day.

Transdermal patches can used to deliver sex hormones, such as testosterone and estrogen, derivatives and analogues or salts thereof of 0.01 to 10 mg per day, particularly, 0.04 to 6 mg/day.

Sex hormones, such as testosterone, testosterone derivatives and analogues or salts thereof may also be provided by means of a buccal gel at a dose of 0.1 mg/day to 100 mg/day. In one embodiment, the dose of testosterone or testosterone derivatives and analogues or salts thereof is a buccal gel is 40 to 80 mg/day. In one class of this embodiment, the dose of testosterone or testosterone derivatives and analogues or salts thereof in a buccal gel is 60 mg/day.

In some embodiments, the methods, systems, and assays described herein can comprise the use of a new target range of free testosterone for ongoing testosterone therapy and free estrogen for estrogen therapy or combined both free testosterone and free estrogen for sex hormone disorders. The new target range arises from the more accurate methods for detecting free testosterone or free estrogen as described herein. Concentration values of free testosterone or free estrogen indicative of low sex hormone concentrations in an individual are also described herein.

Commercial testosterone therapies are known in the art, e.g. topical testosterone formulations (e.g., ANDROGEN™, AXIRON™, FIRST-TESTOSTERONE™, FIRST-TESTOSTERONE MC™, FORTESTA™, and TESTIM™); transdermal patch formulations (e.g., ANDRODERM™); and buccal testosterone formulations (e.g., STRIANT™). Some of the commercial estrogen therapies include conjugated estrogens (Premarin); esters (Amnestrogen, Estratab, Innofem), estraodiol acetate and estropipate.

In some embodiments of the various aspects described herein, a subject's dose of testosterone therapy is adjusted until their free testosterone levels are normal. In some embodiments of the various aspects described herein, a subject's dose of estrogen therapy is adjusted until their free estrogen levels are normal. In some embodiments, a normal free testosterone level can be a level greater than about 100 pg/mL (e.g. as measured using the assays of FIGS. 5A-5C and Example 5). In some embodiments, a normal free testosterone level can be a level greater than about 105 pg/mL (e.g. as measured using the assays of FIGS. 5A-5C and Example 5). In some embodiments, a normal free testosterone level can be a level greater than about 110 pg/mL (e.g. as measured using the assays of FIGS. 5A-5C and Example 5). In some embodiments, a normal free testosterone level can be a level greater than about 114 pg/mL (e.g. as measured using the assays of FIGS. 5A-5C and Example 5). In some embodiments, a normal free testosterone level can be a level greater than about 114.6 pg/mL (e.g. as measured using the assays of FIGS. 5A-5C and Example 5).

In some embodiments of the various aspects described herein, a subject's dose of testosterone therapy is adjusted until their free testosterone levels are in the target range of free testosterone. In some embodiments of the various aspects described herein, a subject's dose of estrogen therapy is adjusted until their free estrogen levels are in the target range of free estrogen. In some embodiments, the total estrogen levels in men is in the range of 10 to 60 pg/mL. In some embodiments the total estrogen levels in women in range of 10-3000 pg/mL depending on age, menopause and/or pregnancy status. In some embodiments, the target range of free testosterone can be a level from about 120 pg/mL to about 375 pg/mL (e.g. as measured using the assays of FIGS. 5A-5C and Example 5). In some embodiments, the target range of free testosterone can be a level from about 140 pg/mL to about 350 pg/mL (e.g. as measured using the assays of 5A-5C and Example 5). In some embodiments, the target range of free testosterone in men can be a level from about 150 pg/mL to about 340 pg/mL (e.g. as measured using the assays of FIGS. 5A-5C and Example 5). In some embodiments, the target range of free testosterone can be a level from about 160 pg/mL to about 320 pg/mL (e.g. as measured using the assays of FIGS. 5A-5C and Example 5). In some embodiments, the target range of free testosterone can be a level from about 164 pg/mL to about 314 pg/mL (e.g. as measured using the assays of FIGS. 5A-5C and Example 5).

The term “sample” or “test sample” as used herein denotes a sample taken or isolated from a biological organism, e.g., a blood or plasma sample from a subject. Exemplary biological samples include, but are not limited to, a biofluid sample; serum, plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, the test sample can be a blood sample. In some embodiments, the test sample can be a plasma sample.

The test sample can be obtained by removing a sample from a subject but can also be accomplished by using previously sample (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject. In some embodiments, the subject can be a subject in need of treatment for (e.g. having or diagnosed as having) a sex hormone disorder. In some embodiments, the subject can be a subject in need of treatment for (e.g. having or diagnosed as having) an androgen disorder. In some embodiments, the subject can be a subject in need of treatment for (e.g. having or diagnosed as having) an estrogen disorder.

In one aspect, described herein is a computer implemented method for an assay, comprising: on a device having one or more processors and a memory storing one or more programs for execution by the one or more processors, the one or more programs including instructions for: a) receiving data from measuring i) a total SHBG concentration, ii) a total sex hormone concentration, such as testosterone or estrogen concentration, and iii) a total albumin concentration in a biological sample obtained from an individual, to determine free sex hormone concentration, such as free testosterone or estrogen concentration, from the individual; b) attributing at least two distinct interconverting microstates of an unliganded SHBG dimer having a first monomer and a second monomer; and c) calculating the free sex hormone concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer. In some embodiments, step b) is performed according to FIGS. 2, 3, 5, and 7. In some embodiments, step c) is performed according to FIG. 7 or Example 5. In some embodiments, the assay further comprises the step of determining the concentration of at least one steroid other than the sex hormone being measured. In some embodiments, the steroid other than the sex hormone being measured is selected from the group consisting of testosterone, an estradiol steroid, an estrone steroid, and a dihydrotestosterone steroid. In some embodiments, the total SHBG concentration, the total sex hormone concentration, and the total albumin concentration is determined using an assay selected from the group consisting of an immunoassay, a binding assay, and a mass-spectrometry assay.

In one aspect, described herein is a computer system for an assay, comprising: one or more processors; and memory to store: one or more programs, the one or more programs comprising: instructions for: a) receiving data from measuring i) a total SHBG concentration, ii) a total sex hormone concentration, such as testosterone or estrogen concentration, and iii) a total albumin concentration in a biological sample obtained from an individual, to determine free sex hormone concentration, such as testosterone or estrogen concentration, from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer. In some embodiments, step b) is performed according to FIGS. 2, 3, 5, and 7. In some embodiments, step c) is performed according to FIG. 7 or Example 5. In some embodiments, the assay further comprises the step of determining the concentration of at least one steroid other than the sex hormone being measured. In some embodiments, the steroid other than the sex hormone being measured is selected from the group consisting of testosterone, an estradiol steroid, an estrone steroid, and a dihydrotestosterone steroid. In some embodiments, the total SHBG concentration, the total sex hormone concentration, and the total albumin concentration is determined using an assay selected from the group consisting of an immunoassay, a binding assay, and a mass-spectrometry assay.

In one aspect, described herein is a non-transitory computer-readable storage medium storing one or more programs for treating an individual suspected of having an sex hormone disorder, the one or more programs for execution by one or more processors of a computer system, the one or more programs comprising instructions for: a) receiving data from measuring i) a total SHBG concentration, ii) a total sex hormone concentration, such as testosterone or estrogen concentration, and iii) a total albumin concentration in a biological sample obtained from an individual, to determine free sex hormone concentration, such as free testosterone or estrogen concentration from the individual; b) attributing at least two distinct interconverting microstates of an unliganded SHBG dimer having a first monomer and a second monomer by applying the New Multi-Step Dynamic Binding Model with Complex Allostery to the data of step a); and c) calculating the free sex hormone concentration in the individual using the New Multi-Step Dynamic Binding Model with Complex Allostery encompassing readjustment of a first equilibria between the microstates upon binding of a first sex hormone molecule to the first monomer and an allosteric interaction between two binding sites of the SHBG dimer. In some embodiments, step b) is performed according to FIGS. 2, 3, 5, and 7. In some embodiments, step c) is performed according to FIG. 7 or Example 5. In some embodiments, the assay further comprises the step of determining the concentration of at least one steroid other than the sex hormone being measured. In some embodiments, the steroid other than the sex hormone being measured is selected from the group consisting of testosterone, an estradiol steroid, an estrone steroid, and a dihydrotestosterone steroid. In some embodiments, the total SHBG concentration, the total sex hormone concentration, and the total albumin concentration is determined using an assay selected from the group consisting of an immunoassay, a binding assay, and a mass-spectrometry assay.

In one aspect, described herein is a computer implemented method for treating an individual suspected of having an sex hormone disorder, comprising: on a device having one or more processors and a memory storing one or more programs for execution by the one or more processors, the one or more programs including instructions for: a) receiving data from measuring i) a total SHBG concentration, ii) a total sex hormone concentration, such as testosterone or estrogen concentration, and iii) a total albumin concentration in a biological sample obtained from an individual suspected of having a sex hormone disorder, to determine free sex hormone concentration, such as testosterone or estrogen concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using a New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for administering a pharmaceutically effective amount of sex hormone to an individual having a free sex hormone concentration below the lower limit of a normal free sex hormone concentration from a healthy individual (e.g., in one embodiment, the lower limit is 114.6 pg/ml); and c) sending a signal for not administering a pharmaceutically effective amount of sex hormone to an individual having a free sex hormone concentration above the lower limit of the normal free sex hormone concentration from a healthy individual (e.g., in one embodiment, the lower limit is 114.6 pg/ml). One of skill in the art understands that the lower limit of a normal free sex hormone concentration will vary according to assay used to detect total sex hormone, SHBG, and albumin and will vary in a different patient populations. Thus the lower limit used for implementation should correspond to the lower limit obtained in a healthy individual using the same assays as used on the biological sample from the test individual, and the lower limit used should be the lower limit from a healthy individual of the same age and type of the test individual. Thus, the computer systems and media will result in sending a signal for administering to an individual having a free sex hormone concentration below the lower limit of the normal in that assay, or sending a signal for not administering to an individual having a free sex hormone concentration above the lower limit of that assay.

In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder is a hypestrogenic disorder selected from the group consisting of an gynecomastia, amenorrhea, ovarian dysfunction, aromatase gene disorder, infertility, subfertility and a pubertal disorder. In some embodiments, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the method further comprises the step of classifying the individual into categories based on additional clinical symptoms.

In one aspect, described herein is a computer system for treating an individual suspected of having an sex hormone disorder, comprising: one or more processors; and memory to store: one or more programs, the one or more programs comprising: instructions for: a) receiving data from measuring i) a total SHBG concentration, ii) a total sex hormone concentration, such as total testosterone or estrogen concentration, and iii) a total albumin concentration in a biological sample obtained from an individual suspected of having an sex hormone disorder, to determine free sex hormone concentration, such as testosterone or estrogen concentration, from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer, d) sending a signal for administering to a pharmaceutically effective amount of sex hormone to an individual having a free sex hormone concentration (based on a, b, c, d above or obtained otherwise) below the lower limit of a normal free sex hormone concentration from a healthy individual (e.g., in one embodiment, the lower limit is 114.6 pg/ml); and e) sending a signal for not administering a pharmaceutically effective amount of sex hormone to an individual having a free sex hormone concentration (based on a, b, c, d above or obtained otherwise) above the lower limit of the normal free sex hormone concentration from a healthy individual (e.g., in one embodiment, the lower limit is 114.6 pg/ml). One of skill in the art understands that the lower limit of a normal free sex hormone concentration will vary according to assay used to detect total sex hormone, SHBG, and albumin and will vary in a different patient populations. Thus the lower limit used for implementation should correspond to the lower limit obtained in a healthy individual using the same assays as used on the biological sample from the test individual, and the lower limit used should be the lower limit from a healthy individual of the same age and type of the test individual. Thus, the computer systems and media will result in sending a signal for administering to an individual having a free sex hormone concentration below the lower limit of the normal in that assay, or sending a signal for not administering to an individual having a free sex hormone concentration above the lower limit of that assay.

In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder arising from and/or characterized by abnormal levels of estrogen. In some embodiments, the estrogen disorder is selected from the group consisting of either an estrogen excess or estrogen deficiency, including but not necessarily limited to ovarian dysfunction, an estrogen expressing tumor, and gynecomastia. In some embodiments, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the system further comprises the step of classifying the individual into categories based on additional clinical symptoms.

In one aspect, described herein is a non-transitory computer-readable storage medium storing one or more programs for treating an individual suspected of having an sex hormone disorder, the one or more programs for execution by one or more processors of a computer system, the one or more programs comprising instructions for a) receiving data from measuring i) a total SHBG concentration, ii) a total sex hormone concentration, such as testosterone or estrogen concentration, and iii) a total albumin concentration in a biological sample obtained from an individual suspected of having an sex hormone disorder, to determine free sex hormone concentration, such as testosterone or estrogen concentration, from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for administering a pharmaceutically effective amount of sex hormone to an individual having a free sex hormone concentration below the lower limit of a normal free sex hormone concentration from a healthy individual (e.g., in one embodiment, below 114.6 pg/ml); and c) sending a signal for not administering a pharmaceutically effective amount of sex hormone to an individual having a free sex hormone concentration above the lower limit of the normal free sex hormone concentration from a healthy individual (e.g., in one embodiment, above 114.6 pg/ml, the lower limit of this assay). One of skill in the art understands that the lower limit of a normal free sex hormone concentration will vary according to assay used to detect total sex hormone, SHBG, and albumin and will vary in a different patient populations. Thus the lower limit used for implementation should correspond to the lower limit obtained in a healthy individual using the same assays as used on the biological sample from the test individual, and the lower limit used should be the lower limit from a healthy individual of the same age and type of the test individual. Thus, the computer systems and media will result in sending a signal for administering to an individual having a free sex hormone concentration below the lower limit of the normal in that assay, or sending a signal for not administering to an individual having a free sex hormone concentration above the lower limit of that assay.

In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder arising from and/or characterized by abnormal levels of estrogen. In some embodiments, the estrogen disorder is selected from the group consisting of either an estrogen excess or estrogen deficiency, including but not necessarily limited to ovarian dysfunction, an estrogen expressing tumor, gynecomastia, and infertility. In some embodiments, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the medium further comprises the step of classifying the individual into categories based on additional clinical symptoms.

In one aspect, described herein is a computer implemented method for determining a need for adjustment of a dose of sex hormone, such as testosterone administered to an individual with hypogonadism, androgen deficiency syndrome, or any other condition for which testosterone therapy is indicated, comprising: on a device having one or more processors and a memory storing one or more programs for execution by the one or more processors, the one or more programs including instructions for: a) receiving data from determining the concentration of free sex hormone, such as free testosterone or estrogen, in an individual receiving sex hormone therapy at a first dose, wherein the concentration of free sex hormone, such as testosterone or estrogen, is determined by measuring i) a total SHBG concentration, ii) a total sex hormone concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer and; d) sending a signal for providing a second (adjusted) dose of sex hormone that is higher than the first dose when the free sex hormone concentration is below the lower end of the target therapeutic (e.g. 164 pg/ml); and e) sending a signal for providing a second (adjusted) dose of sex hormone that is lower than the first dose when the free sex hormone concentration is above the upper end of the target therapeutic range (e.g. 314 pg/ml). One of skill in the art understands that the target therapeutic range could vary depending on the age of the patient, the population, and the types of total sex hormone. SHBG and albumin assays, in which case sending a signal sending a signal for providing a second (adjusted) dose of sex hormone that is higher than the first dose when the free sex hormone concentration is below the target therapeutic range for that age, population, and assay of total sex hormone, SHBG and albumin, or sending a signal for providing a second (adjusted) dose of sex hormone that is lower than the first dose when the free sex hormone concentration is above the target therapeutic range. Examples of suitable target ranges are described herein.

In some embodiments, the method can further comprise the step of receiving data of the first dose of testosterone administered to the individual. In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder arising from and/or characterized by abnormal levels of estrogen. In some embodiments, the estrogen disorder is selected from the group consisting of either an estrogen excess or estrogen deficiency, including but not necessarily limited to ovarian dysfunction, an estrogen expressing tumor, gynecomastia, and infertility, subfertility, and pubertal disorder. In some embodiments, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the method further comprises the step of classifying the individual into categories based on additional clinical symptoms.

In one aspect, described herein is a computer system for determining a need for adjustment of a dose of sex hormone, such as testosterone or estrogen, administered to an individual, comprising: one or more processors; and memory to store: one or more programs, the one or more programs comprising: instructions for: a) receiving data from determining the concentration of free sex hormone, such as free testosterone or free estrogen, in an individual receiving sex hormone therapy at a first dose, wherein the concentration of free sex hormone is determined by measuring i) a total SHBG concentration, ii) a total sex hormone concentration, such as total testosterone or estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer; d) sending a signal for providing a second dose of sex hormone that is higher than the first dose when the free sex hormone concentration is below the lower end of the target therapeutic range (e.g. 164 pg/ml), and e) sending a signal for providing a second dose of sex hormone that is lower than the first dose when the free testosterone concentration is above the upper end of the target therapeutic range (e.g. 314 pg/ml).

In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder arising from and/or characterized by abnormal levels of estrogen. In some embodiments, the estrogen disorder is selected from the group consisting of either an estrogen excess or estrogen deficiency, including but not necessarily limited to ovarian dysfunction, an estrogen expressing tumor, gynecomastia, and infertility, subfertility, and pubertal disorder. In some embodiments, the individual has been diagnosed with a disease selected from the group consisting of: diabetes, human immunodeficiency virus (HIV), hepatitis B, hepatitis C, hypothyroidism or hyperthyroidism, estrogen insensitivity, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the method further comprises the step of classifying the individual into categories based on additional clinical symptoms.

In one aspect, described herein is a non-transitory computer-readable storage medium storing one or more programs for determining a need for adjustment of a dose of sex hormone, such as testosterone or estrogen, administered to an individual, the one or more programs for execution by one or more processors of a computer system, the one or more programs comprising instructions for: a) receiving data from determining the concentration of free sex hormone, such as free testosterone or free estrogen, in an individual receiving sex hormone therapy at a first dose, wherein the concentration of free sex hormone is determined by measuring i) a total SHBG concentration, ii) a total sex hormone concentration, such as total testosterone or estrogen concentration, and iii) a total albumin concentration in a biological sample obtained from an individual, to determine free sex hormone concentration from the individual; b) attributing at least two distinct interconverting microstates of an unliganded SHBG dimer having a first monomer and a second monomer by applying the New Multi-Step Dynamic Binding Model with Complex Allostery to the data of step a); c) calculating the free sex hormone concentration in the individual using the New Multi-step Dynamic Binding Model with Complex Allostery encompassing readjustment of a first equilibria between the microstates upon binding of a first sex hormone molecule to the first monomer and an allosteric interaction between two binding sites of the SHBG dimer, d) sending a signal for providing a second dose of sex hormone that is higher than the first dose when the free sex hormone concentration is below the lower end of the target therapeutic range (e.g. 164 pg/ml); and e) sending a signal for providing a second dose of sex hormone that is lower than the first dose when the free sex hormone concentration is above the upper end of the target therapeutic range (e.g. 314 pg/ml). In specification: In another embodiment, the target therapeutic range could vary with the age of the patient, co-morbid conditions, and the types of assays used for measuring total sex hormone, SHBG and albumin. in which case sending the signal for providing a second dose of sex hormone that is higher than the first dose when the free sex hormone concentration is below the lower end of the target therapeutic range for that age, co-morbid conditions and assay.

In some embodiments, step c) is performed according to FIGS. 2, 3, 5, and 7. In some embodiments, step d) is performed according to FIG. 7 or Example 5. In some embodiments, the individual is a male over the age of 35. In some embodiments, the individual is a female over the age of 35. In some embodiments, the androgen disorder is selected from the group consisting of a testosterone deficiency, an androgen deficiency, a hyperandrogenic disorder, an androgen expressing tumor, and a hypogonadism disorder. In some embodiments, the androgen disorder is a hyperandrogenic disorder selected from the group consisting of an acne disorder, a hirsutism disorder, and an androgenic alopecia disorder. In some embodiments, the estrogen disorder arising from and/or characterized by abnormal levels of estrogen. In some embodiments, the estrogen disorder is selected from the group consisting of either an estrogen excess or estrogen deficiency, including but not necessarily limited to ovarian dysfunction, an estrogen expressing tumor, gynecomastia, and infertility, subfertility, and pubertal disorder. In some embodiments, the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered. In some embodiments, the method further comprises the step of classifying the individual into categories based on additional clinical symptoms.

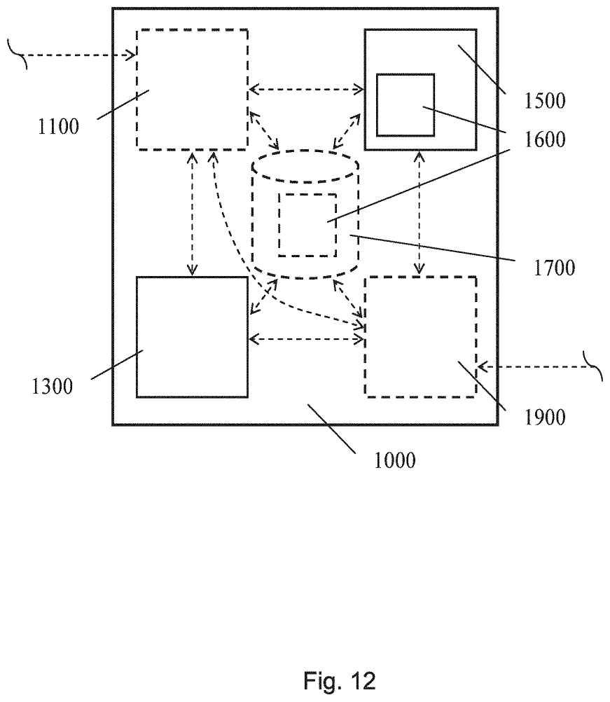

FIG. 12 depicts a device or a computer system 1000 comprising one or more processors 1300 and a memory 1500 storing one or more programs 1600 for execution by the one or more processors 1300. In some embodiments, the device or computer system 1000 can further comprise a non-transitory computer-readable storage medium 1700 storing the one or more programs 1600 for execution by the one or more processors 1300 of the device or computer system 1000.

In some embodiments, the device or computer system 1000 can further comprise one or more input devices 1100, which can be configured to send or receive information to or from any one from the group consisting of: an external device (not shown), the one or more processors 1300, the memory 1500, the non-transitory computer-readable storage medium 1700, and one or more output devices 1900.

In some embodiments, the device or computer system 1000 can further comprise one or more output devices 1900, which can be configured to send or receive information to or from any one from the group consisting of: an external device (not shown), the one or more processors 1300, the memory 1500, and the non-transitory computer-readable storage medium 1700.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology. CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

In some embodiments of any of the aspects described herein, instead of steps a-c, the data received is a previously calculated concentration of free sex hormone, such as free testosterone or free estrogen.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The term “effective amount” as used herein refers to the amount of, e.g. sex hormone, needed to alleviate at least one or more symptom of the disease or disorder, and relates to a sufficient amount of pharmacological composition to provide the desired effect. The term “therapeutically effective amount” therefore refers to an amount of, e.g., sex hormone that is sufficient to provide a particular anti-low sex hormone effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of testosterone, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for free testosterone as described herein, among others. The dosage can be determined by a physician and adjusted, as necessary, to suit observed effects of the treatment.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein. “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”. “increase”, “enhance”, or “activate” can mean an increase of at least 10% as compared to a reference level, for example an increase of at least about 200/o, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” or “individual” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal. e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of androgen disorders. A subject can be male or female.

A subject can be one who has been previously diagnosed with or identified as suffering from or having a condition in need of treatment (e.g. an sex hormone disorder, such as an androgen disorder or estrogen) or one or more complications related to such a condition, and optionally, have already undergone treatment for an sex hormone disorder or the one or more complications related to an sex hormone disorder. Alternatively, a subject can also be one who has not been previously diagnosed as having an sex hormone disorder or one or more complications related to an sex hormone disorder. For example, a subject can be one who exhibits one or more risk factors for an sex hormone disorder or one or more complications related to an sex hormone disorder or a subject who does not exhibit risk factors. For example, a subject could be treated with sex hormones for infertility through in-vitro fertilization or methods related to fertility treatments.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a sex hormone disorder, such as an androgen disorder or estrogen disorder. The term “treating” includes reducing or alleviating at least one adverse effect or symptom of a condition, disease or disorder associated with an androgen disorder. Treatment is generally “effective” if one or more symptoms or clinical markers are reduced. Alternatively, treatment is “effective” if the progression of a disease is reduced. That is, “treatment” includes not just the improvement of symptoms or markers, but also a slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an.” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.). The Encyclopedia of Molecular Biology, published by Blackwell Science Ltd., 1994 (ISBN 0-632-02182-9); The ELISA guidebook (Methods in molecular biology 149) by Crowther J. R. (2000); Fundamentals of RIA and Other Ligand Assays by Jeffrey Travis, 1979, Scientific Newsletters; Benjamin Lewin, Genes X, published by Jones & Bartlett Publishing, 2009 (ISBN-10: 0763766321); Kendrew et al. (eds.)., Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.). Cold Spring Harbor Laboratory Press, Cold Spring Harbor, N.Y., USA (2012); Davis et al., Basic Methods in Molecular Biology, Elsevier Science Publishing. Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A computer implemented method for an assay, comprising:
on a device having one or more processors and a memory storing one or more programs for execution by the one or more processors, the one or more programs including instructions for:
   a) receiving data from measuring i) a total SHBG concentration, ii) a total testosterone concentration, and iii) a total albumin concentration in a biological sample obtained from an individual, to determine free testosterone concentration from the individual;
   b) attributing at least two distinct interconverting microstates of an unliganded SHBG dimer having a first monomer and a second monomer; and
   c) calculating the free testosterone concentration in the individual using the New Multi-Step Dynamic Binding Model with Complex Allostery encompassing readjustment of a first equilibria between the microstates upon binding of a first testosterone molecule to the first monomer and an allosteric interaction between two binding sites of the SHBG dimer.
2. The computer implemented method of paragraph 1, wherein step b) is performed according to FIGS. 2, 3, 5, and 7.
3. The computer implemented method of paragraph 1, wherein step c) is performed according to FIG. 7 or Example 5.
4. The computer implemented method of paragraph 1, wherein the assay further comprises the step of determining the concentration of at least one non-testosterone steroid.
5. The computer implemented method of paragraph 4, wherein the non-testosterone steroid is selected from the group consisting of an estradiol steroid, an estrone steroid, and a dihydrotestosterone steroid.
6. The computer implemented method of paragraph 1, wherein the total SHBG concentration, the total testosterone concentration, and the total albumin concentration is determined using an assay selected from the group consisting of an immunoassay, a binding assay, and a mass-spectrometry assay.
7. A computer system for an assay, comprising:
   one or more processors; and
   memory to store:
   one or more programs, the one or more programs comprising:
   instructions for:
   a) receiving data from measuring i) a total SHBG concentration, ii) a total testosterone concentration, and iii) a total albumin concentration in a biological sample obtained from an individual, to determine free testosterone concentration from the individual:
   b) attributing at least two distinct interconverting microstates of an unliganded SHBG dimer having a first monomer and a second monomer by applying the New Multi-Step Dynamic Binding Model with Complex Allostery to the data of step a); and
   c) calculating the free testosterone concentration in the individual using the New Multi-Step Dynamic Binding Model with Complex Allostery encompassing readjustment of a first equilibria between the microstates upon binding of a first testosterone molecule to the first monomer and an allosteric interaction between two binding sites of the SHBG dimer.
8. The computer system of paragraph 7, wherein step b) is performed according to FIGS. 2, 3, 5, and 7.
9. The computer system of paragraph 7, wherein step c) is performed according to FIG. 7 or Example 5.
10. The computer system of paragraph 7, wherein the assay further comprises the step of determining the concentration of at least one non-testosterone steroid.
11. The computer system of paragraph 10, wherein the non-testosterone steroid is selected from the group consisting of an estradiol steroid, an estrone steroid, and a dihydrotestosterone steroid.
12. The computer system of paragraph 7, wherein the total SHBG concentration, the total testosterone concentration, and the total albumin concentration is determined using an assay selected from the group consisting of an immunoassay, a binding assay, and a mass-spectrometry assay.
13. A non-transitory computer-readable storage medium storing one or more programs for treating an individual suspected of having an androgen disorder, the one or more programs for execution by one or more processors of a computer system, the one or more programs comprising instructions for:
   a) receiving data from measuring i) a total SHBG concentration, ii) a total testosterone concentration, and iii) a total albumin concentration in a biological sample obtained from an individual, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a); and
   c) calculating the free testosterone concentration in the individual using the New Multi-Step Dynamic Binding Model with Complex Allostery encompassing readjustment of a first equilibria between the microstates upon binding of a first testosterone molecule to the first monomer and an allosteric interaction between two binding sites of the SHBG dimer.
14. The non-transitory computer-readable storage medium of paragraph 13, wherein step b) is performed according to FIGS. 2, 3, 5, and 7.
15. The non-transitory computer-readable storage medium of paragraph 13, wherein step c) is performed according to FIG. 7 and/or Example 5.
16. The non-transitory computer-readable storage medium of paragraph 13, wherein the system further comprises the step of determining the concentration of at least one non-testosterone steroid.
17. The non-transitory computer-readable storage medium of paragraph 16, wherein the non-testosterone steroid is selected from the group consisting of an estradiol steroid, an estrone steroid, and a dihydrotestosterone steroid.
18. The non-transitory computer-readable storage medium of paragraph 13, wherein the total SHBG concentration, the total testosterone concentration, and the total albumin concentration is determined using an assay selected from the group consisting of an immunoassay, a binding assay, and a mass-spectrometry assay.
19. An assay comprising the steps of:
   a) measuring i) a total SHBG concentration, ii) a total testosterone concentration, and iii) a total albumin concentration in a biological sample obtained from an individual, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a); and
   c) calculating the free testosterone concentration in the individual using the New Multi-Step Dynamic Binding Model with Complex Allostery encompassing readjustment of a first equilibria between the microstates upon binding of a first testosterone molecule to the first monomer and an allosteric interaction between two binding sites of the SHBG dimer
20. The assay of paragraph 19, wherein step b) is performed according to FIGS. 2, 3, 5, and 7.
21. The assay of paragraph 19, wherein step c) is performed according to FIG. 7 and/or Example 5.
22. The assay of paragraph 19, wherein the assay further comprises the step of determining the concentration of at least one non-testosterone steroid.
23. The assay of paragraph 22, wherein the non-testosterone steroid is selected from the group consisting of an estradiol steroid, an estrone steroid, and a dihydrotestosterone steroid.
24. The assay of paragraph 19, wherein the total SHBG concentration, the total testosterone concentration, and the total albumin concentration is determined using an assay selected from the group consisting of an immunoassay, a binding assay, and a mass-spectrometry assay.
25. A computer implemented method for treating an individual suspected of having an androgen disorder, comprising:
   on a device having one or more processors and a memory storing one or more programs for execution by the one or more processors, the one or more programs including instructions for:
   a) receiving data from measuring i) a total SHBG concentration, ii) a total testosterone concentration, and iii) a total albumin concentration in a biological sample obtained from an individual suspected of having an androgen disorder, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using a New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   d) sending a signal for administering a pharmaceutically effective amount of testosterone to an individual having a free testosterone concentration below the lower limit of a normal free testosterone concentration from a healthy individual (e.g., in one embodiment, the lower limit is 114.6 pg/ml); and
   e) sending a signal for not administering a pharmaceutically effective amount of testosterone to an individual having a free testosterone concentration above the lower limit of the normal free testosterone concentration from a healthy individual (e.g., in one embodiment, the lower limit is 114.6 pg/ml).
26. The computer implemented method of paragraph 25, wherein step c) is performed according to FIGS. 2, 3, 5, and 7.
27. The computer implemented method of paragraph 25, wherein step d) is performed according to FIG. 7 or Example 5.
28. The computer implemented method of paragraph 25, wherein the individual is a male over the age of 35.
29. The computer implemented method of paragraph 25, wherein the androgen disorder is selected from the group consisting of a testosterone deficiency, an androgen deficiency, a hyperandrogenic disorder, an androgen expressing tumor, and a hypogonadism disorder.
30. The computer implemented method of paragraph 25, wherein the androgen disorder is a hyperandrogenic disorder selected from the group consisting of an acne disorder, a hirsutism disorder, and an androgenic alopecia disorder.
31. The computer implemented method of paragraph 25, wherein the individual has been diagnosed with a disease selected from the group consisting of: diabetes, human immunodeficiency virus (HIV), hepatitis B, hepatitis C, hypothyroidism or hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered.
32. The computer implemented method of paragraph 25, further comprising the step of classifying the individual into categories based on additional clinical symptoms.
33. A computer system for treating an individual suspected of having an androgen disorder, comprising:
   one or more processors; and
   memory to store:
   one or more programs, the one or more programs comprising:
   instructions for:
   a) receiving data from measuring i) a total SHBG concentration, ii) a total testosterone concentration, and iii) a total albumin concentration in a biological sample obtained from an individual suspected of having an androgen disorder, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   d) sending a signal for administering to a pharmaceutically effective amount of testosterone to an individual having a free testosterone concentration below the lower limit of a normal free testosterone concentration from a healthy individual (e.g., in one embodiment, the lower limit is 114.6 pg/ml): and
   e) sending a signal for not administering a pharmaceutically effective amount of testosterone to an individual having a free testosterone concentration above the lower limit of the normal free testosterone concentration from a healthy individual (e.g., in one embodiment, the lower limit is 114.6 pg/ml).
34. The computer system of paragraph 33, wherein step c) is performed according to FIGS. 2, 3, 5, and 7.
35. The computer system of paragraph 33, wherein step d) is performed according to FIG. 7 or Example 5.
36. The computer system of paragraph 33, wherein the individual is a male over the age of 35.
37. The computer system of paragraph 33, wherein the androgen disorder is selected from the group consisting of a testosterone deficiency, an androgen deficiency, a hyperandrogenic disorder, and a hypogonadism disorder.
38. The computer system of paragraph 33, wherein the androgen disorder is a hyperandrogenic disorder selected form the group consisting of an acne disorder, a hirsutism disorder, an androgen expressing tumor, and an androgenic alopecia disorder.
39. The computer system of paragraph 33, wherein the individual has been diagnosed with a disease selected from the group consisting of: diabetes, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, and obesity, and other conditions in which SHBG or albumin concentrations are altered.
40. The computer system of paragraph 33, further comprising the step of classifying the individual into categories based on additional clinical symptoms.
41. A non-transitory computer-readable storage medium storing one or more programs for treating an individual suspected of having an androgen disorder, the one or more programs for execution by one or more processors of a computer system, the one or more programs comprising instructions for:
   a) receiving data from measuring i) a total SHBG concentration, ii) a total testosterone concentration, and iii) a total albumin concentration in a biological sample obtained from an individual suspected of having an androgen disorder, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using New Multi-step Dynamic Binding Model with Complex Allostery encompassing readjustment of a first equilibria between the microstates upon binding of a first testosterone molecule to the first monomer and an allosteric interaction between two binding sites of the SHBG dimer:
   d) sending a signal for administering a pharmaceutically effective amount of testosterone to an individual having a free testosterone concentration below the lower limit of a normal free testosterone concentration from a healthy individual (e.g., in one embodiment, below 114.6 pg/ml): and
   e) sending a signal for not administering a pharmaceutically effective amount of testosterone to an individual having a free testosterone concentration above the lower limit of the normal free testosterone concentration from a healthy individual (e.g., in one embodiment, above 114.6 pg/ml, the lower limit of this assay).
42. The non-transitory computer-readable storage medium of paragraph 41, wherein step c) is performed according to FIGS. 2, 3, 5, and 7.
43. The non-transitory computer-readable storage medium of paragraph 41, wherein step d) is according to FIG. 7 or Example 5.
44. The non-transitory computer-readable storage medium of paragraph 41, wherein the individual is a male over the age of 35.
45. The non-transitory computer-readable storage medium of paragraph 41, wherein the androgen disorder is selected from the group consisting of a testosterone deficiency, an androgen deficiency, a hyperandrogenic disorder, and a hypogonadism disorder.
46. The non-transitory computer-readable storage medium of paragraph 41, wherein the androgen disorder is a hyperandrogenic disorder selected form the group consisting of an acne disorder, a hirsutism disorder, an androgen expressing tumor, and an androgenic alopecia disorder.
47. The non-transitory computer-readable storage medium of paragraph 41, wherein the individual has been diagnosed with a disease selected from the group consisting of: diabetes, human immunodeficiency virus (HIV), hypothyroidism, hyperthyroidism, hepatitis B, hepatitis C, androgen insensitivity, acromegaly, anorexia, muscular dystrophy, liver disease, cancer cachexia, malnutrition, nephrotic syndrome, and obesity.
48. The non-transitory computer-readable storage medium of paragraph 41, further comprising the step of classifying the individual into categories based on additional clinical symptoms.
49. A method for treating an individual suspected of having an androgen disorder comprising:
   a) administering a pharmaceutically effective amount of testosterone to an individual who has had a free testosterone level determined by measuring i) a total SHBG concentration, ii) a total testosterone concentration, and iii) a total albumin concentration in a biological sample from the individual suspected of having an androgen disorder;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using an the New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   d) administering a pharmaceutically effective amount of testosterone to an individual having a free testosterone concentration below the lower limit of a normal free testosterone concentration from a healthy individual (e.g., in one embodiment, below 114.6 pg/ml); and
   e) not administering a pharmaceutically effective amount of testosterone to an individual having a free testosterone concentration above the lower limit of the normal free testosterone concentration from a healthy individual (e.g., in one embodiment, above 114.6 pg/ml, the lower limit of this assay).
50. The method of paragraph 49, wherein step c) is performed according to FIGS. 2, 3, 5, and 7.
51. The method of paragraph 49, wherein step d) is performed according to FIG. 7 or Example 5.
52. The method of paragraph 49, wherein the individual is a male over the age of 35.
53. The method of paragraph 49, wherein the androgen disorder is selected from the group consisting of a testosterone deficiency, an androgen deficiency, and a hypogonadism disorder.
54. The method of paragraph 49, wherein the individual has been diagnosed with a disease selected from the group consisting of: diabetes, obesity, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, obesity, and other conditions in which SHBG or albumin concentrations are altered.
55. The method of paragraph 49, further comprising the step of classifying the individual into categories based on additional clinical symptoms.
56. A computer implemented method for determining a need for adjustment of a dose of testosterone administered to an individual with hypogonadism, androgen deficiency syndrome, or any other condition for which testosterone therapy is indicated, comprising: on a device having one or more processors and a memory storing one or more programs for execution by the one or more processors, the one or more programs including instructions for:
   a) receiving data from determining the concentration of free testosterone in an individual receiving testosterone therapy at a first dose, wherein the concentration of free testosterone is determined by measuring i) a total SHBG concentration, ii) a total testosterone concentration, and iii) a total albumin concentration in a biological sample obtained from an individual, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer and;
   d) sending a signal for providing a second (adjusted) dose of testosterone that is higher than the first dose when the free testosterone concentration is below the lower end of the target therapeutic (e.g. 164 pg/ml); and
   e) sending a signal for providing a second (adjusted) dose of testosterone that is lower than the first dose when the free testosterone concentration is above the upper end of the target therapeutic range (e.g. 314 pg/ml).
57. The computer implemented method of paragraph 56, further comprising the step of receiving data of the first dose of testosterone administered to the individual.
58. The computer implemented method of paragraph 56, wherein step b) is performed according to FIGS. 2, 3, 5, and 7.
59. The computer implemented method of paragraph 56, wherein step c) is performed according to FIG. 7 or Example 5.
60. The computer implemented method of paragraph 56, wherein the individual is a male over the age of 35.
61. The computer implemented method of paragraph 56, wherein the individual has a disorder selected from the group consisting of a testosterone deficiency, an androgen deficiency, and a hypogonadism disorder.
62. The computer implemented method of paragraph 56, wherein the individual has been diagnosed with a disease selected from the group consisting of: diabetes, obesity, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, obesity, and other conditions in which SHBG or albumin concentrations are altered.
63. A computer system for determining a need for adjustment of a dose of testosterone administered to an individual, comprising:
   one or more processors; and
   memory to store:
   one or more programs, the one or more programs comprising:
   instructions for:
   a) receiving data from determining the concentration of free testosterone in an individual receiving testosterone therapy at a first dose, wherein the concentration of free testosterone is determined by measuring i) a total SHBG concentration, ii) a total testosterone concentration, and iii) a total albumin concentration in a biological sample obtained from an individual, to determine free testosterone concentration from the individual;
   b) attributing at least two distinct interconverting microstates of an unliganded SHBG dimer having a first monomer and a second monomer by applying the New Multi-Step Dynamic Binding Model with Complex Allostery to the data of step a);
   c) calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   d) sending a signal for providing a second dose of testosterone that is higher than the first dose when the free testosterone concentration is below the lower end of the target therapeutic range (e.g. 164 pg/ml); and
   e) sending a signal for providing a second dose of testosterone that is lower than the first dose when the free testosterone concentration is above the upper end of the target therapeutic range (e.g. 314 pg/ml).
64. The computer implemented method of paragraph 63, wherein step c) is performed according to FIGS. 2, 3, 5, and 7.
65. The computer implemented method of paragraph 63, wherein step d) is performed according to FIG. 7 or Example 5.
66. The computer implemented method of paragraph 63, wherein the individual is a male over the age of 35.
67. The computer implemented method of paragraph 63, wherein the individual has a disorder selected from the group consisting of a testosterone deficiency, an androgen deficiency, and a hypogonadism disorder, or any condition for which testosterone therapy is indicated or may be given.
68. The computer implemented method of paragraph 63, wherein the androgen disorder is a hyperandrogenic disorder selected form the group consisting of an acne disorder, a hirsutism disorder, an androgen expressing tumor, and an androgenic alopecia disorder.
69. The computer implemented method of paragraph 63, wherein the individual has been diagnosed with a disease selected from the group consisting of: diabetes, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, obesity, and any condition in which SHBG or albumin concentrations are altered.
70. A non-transitory computer-readable storage medium storing one or more programs for determining a need for adjustment of a dose of testosterone administered to an individual, the one or more programs for execution by one or more processors of a computer system, the one or more programs comprising instructions for:
   a. receiving data from determining the concentration of free testosterone in an individual receiving testosterone therapy at a first dose, wherein the concentration of free testosterone is determined by measuring i) a total SHBG concentration, ii) a total testosterone concentration, and iii) a total albumin concentration in a biological sample obtained from an individual, to determine free testosterone concentration from the individual:
   b. attributing at least two distinct interconverting microstates of an unliganded SHBG dimer having a first monomer and a second monomer by applying the New Multi-Step Dynamic Binding Model with Complex Allostery to the data of step a);
   c. calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   d. sending a signal for providing a second dose of testosterone that is higher than the first dose when the free testosterone concentration is below the lower end of the target therapeutic range (e.g. 164 pg/ml); and
   e. sending a signal for providing a second dose of testosterone that is lower than the first dose when the free testosterone concentration is above the upper end of the target therapeutic range (e.g. 314 pg/ml).
71. The non-transitory computer-readable storage medium of paragraph 70, wherein step c) is performed according to FIGS. 2, 3, 5, and 7.
72. The non-transitory computer-readable storage medium of paragraph 70, wherein step d) is performed according to FIG. 7 or Example 5.
73. The non-transitory computer-readable storage medium of paragraph 70, wherein the individual is a male over the age of 35.
74. The non-transitory computer-readable storage medium of paragraph 70, wherein the individual has a disorder selected from the group consisting of a testosterone deficiency, an androgen deficiency, and a hypogonadism disorder, or any other condition in which testosterone therapy may be given or is indicated.
75. The non-transitory computer-readable storage medium of paragraph 70, wherein the individual has been diagnosed with a disease selected from the group consisting of: diabetes, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, obesity, and any condition in which SHBG or albumin concentrations are altered.
76. A method for determining a need for adjustment of a dose of testosterone administered to an individual comprising
   a) determining the concentration of free testosterone in an individual receiving testosterone therapy at a first dose, wherein the concentration of free testosterone is determined by
   b) measuring i) a total SHBG concentration, ii) a total testosterone concentration, and iii) a total albumin concentration in a biological sample obtained from an individual, to determine free testosterone concentration from the individual;
   c) attributing at least two distinct interconverting microstates of an unliganded SHBG dimer having a first monomer and a second monomer by applying the New Multi-Step Dynamic Binding Model with Complex Allostery to the data of steps a) and b):
   d) calculating the free testosterone concentration in the individual using the New Multi-step Dynamic Binding Model with Complex Allostery encompassing readjustment of a first equilibria between the microstates upon binding of a first testosterone molecule to the first monomer and an allosteric interaction between two binding sites of the SHBG dimer;
   e) providing a second dose of testosterone that is higher than the first dose when the free testosterone concentration is below the lower end of the target therapeutic range (e.g. 164 pg/ml); and
   f) providing a second dose of testosterone that is lower than the first dose when the free testosterone concentration is above the upper end of the target therapeutic range (314 pg/ml).
77. The method of paragraph 76, wherein step c) is performed according to FIGS. 2, 3, 5, and 7.
78. The method of paragraph 76, wherein step d) is performed according to FIG. 7 or Example 5.
79. The method of paragraph 76, wherein the individual is a male over the age of 35.
80. The method of paragraph 76, wherein the individual has a disorder selected from the group consisting of a testosterone deficiency, an androgen deficiency, and a hypogonadism disorder.
81. The method of paragraph 76, wherein the individual has been diagnosed with a disease selected from the group consisting of: diabetes, human immunodeficiency virus (HIV), hepatitis B, hepatitis C, hypothyroidism, hyperthyroidism, androgen insensitivity, acromegaly, anorexia, muscular dystrophy, liver disease, cancer cachexia, malnutrition, nephrotic syndrome, and obesity, or any other condition in which SHBG and/or albumin concentrations.
82. The method of paragraph 76, wherein the method comprises the step of: using the free testosterone concentration determined using the new Multistep Dynamic Binding Model with Complex Allostery to determine the dose or to individually adjust the dose of a formulation of testosterone for the treatment of a medical disease, taking into account patient's age, body weight and body mass index, medical conditions, including any co-morbid conditions, albumin and SHBG, and/or LH and FSH concentrations, and other patient-specific factors.

### EXAMPLES

### Example 1: Determination of Free Sex Steroid Concentration

In Conditions Characterized by Alterations in Sex-Hormone Binding Globulin (SHBG), total testosterone (TT) may not reflect the androgen status accurately and estimates of free testosterone (FT) are needed. Equations based on the prevailing model of SHBG:T interaction, although known to be systematically erroneous, have been used widely to calculate free testosterone.

Methods. Free testosterone concentrations calculated using a law-of-mass-action equation differ substantially from those measured using equilibrium dialysis. We investigated the dynamics of testosterone and SHBG interaction using binding isotherms, SHBG depletion curves, and isothermal titration calorimetry (ITC). The FT values calculated from new model derived from these experiments were compared to equilibrium dialysis using samples from randomized testosterone trials in men and women.

Results. Binding isotherms generated by incubating 5, 10 or 20 nM SHBG with 0-to-400 nM testosterone could not be explained by the prevailing model developed by Vermeulen et al. (1). Comprehensive evaluation of data derived from binding isotherms, ligand depletion curves, and ITC suggested a complex association of testosterone with SHBG dimer; the new Multi-step Dynamic Binding Model with Complex Allostery that provided the best fit to these data encompasses at least two inter-converting microstates in unliganded SHBG, readjustment of equilibria between unliganded states upon binding of the first ligand molecule, and allosteric interaction between two binding sites of SHBG dimer. In samples from testosterone trials in men and women, free testosterone predicted using the new Multi-step Dynamic Binding Model with Complex Allostery did not systematically differ from that measured using equilibrium dialysis.

The new Multi-step Dynamic Binding Model with Complex Allostery of testosterone's binding to SHBG provides excellent fit to experimental data derived from binding isotherm, depletion curves and ITC. Free testosterone concentrations calculated using the new Multi-step Dynamic Binding Model with Complex Allostery closely match those measured using equilibrium dialysis.

Testosterone, the major androgen in humans, circulates in blood bound largely to sex hormone binding globulin (SHBG) and albumin. Testosterone can also bind to orosomucoid and transcortin proteins. According to the free hormone hypothesis, only the unbound or free fraction—0.5 to 3.0% of total—can cross the plasma membrane and is biologically active. In many conditions that affect SHBG concentrations, such as obesity, diabetes, aging, hyperthyroidism, liver disease, acromegaly, and HIV-infection, total testosterone concentrations are altered because of the changes in SHBG concentrations; in these conditions, determination of free testosterone concentration is needed to obtain an accurate assessment of androgen status.

A number of direct and indirect methods—equilibrium dialysis, ultrafiltration, tracer analog methods, and calculations based on homogenous SHBG:T binding equation—for the determination of free testosterone have been published (2-8). The accuracy of the available assays for the measurement of free testosterone has engendered concern (3). Equilibrium dialysis is widely considered the reference method, but the method is cumbersome, affected by dialysis conditions and tracer impurities, and has lower precision (3). The tracer analog methods are convenient but inaccurate (9). Bioavailable testosterone levels (the unbound testosterone plus testosterone bound to albumin) can be measured by the ammonium sulfate precipitation method (9-12), but the methodological difficulties in performing these assays led the Endocrine Society's Expert Panel to shy away from recommending its use outside of research laboratories (3). Recognizing these methodological difficulties in the measurement of free testosterone, the Endocrine Society's Expert Panel suggested that “the calculation of free testosterone concentration from reliably measured total testosterone and SHBG using mass action equations provides the best approach for the estimation of free testosterone concentration (3)”. Therefore, algorithms for calculating free testosterone from total testosterone, SHBG and albumin concentrations using the law-of-mass-action equations (1, 13-16) or empirically-derived equations (13-16) have been published, advocated, and used widely.

The current equations based on homogenous SHBG:T interaction (equal affinity of T for each of the monomers within SHBG dimer and without allostery in SHBG dimers) proposed by Vermeulen and others (13, 17) are based on the assumption that each SHBG dimer binds two testosterone molecules, and that each of the two binding sites on SHBG dimer has similar binding constants (data not shown). It is demonstrated herein that the current model of testosterone binding to SHBG that has formed the basis of the law-of-mass-action equation is erroneous; the free testosterone levels derived from these equations display substantial discrepancy from the values obtained by equilibrium dialysis (18). Based on binding isotherms, ligand depletion experiments, and isothermal titration calorimetry (ITC), we provide experimental evidence of complex allostery between the binding sites on the two SHBG monomers in the presence of the ligand. Based on this new model of testosterone binding to SHBG, described herein is a novel algorithm for the calculation of free testosterone, applied it to samples derived from randomized testosterone trials in men and women, and compared the resus with those obtained using equilibrium dialysis.

Materials and Methods. Human SHBG purified from serum (The Binding Site Group, Ltd Birmingham. UK cat # BH089.X) was characterized by protein gel denaturation-renaturation experiments and by measuring its ability to bind testosterone. Testosterone standard 1.0 mg/mL±2% (3.47 mM) was obtained from Cerilliant (Round Rock Tex.).

Equilibrium dialysis was performed in 96-Well equilibrium dialysis chambers with 10 kDa molecular weight cut-off (Harvard Apparatus, Holliston, Mass. cat #742331). Equilibrium dialysis buffer contained 30 mM HEPES buffer pH 7.4, 90 mM NaCl, 1 mM MgSO4, 187 uM CaCl₂ in ultrapure water. For binding and depletion assays, SHBG was reconstituted in equilibrium dialysis buffer. Testosterone concentration was measured using a liquid chromatography tandem mass spectrometry (LC-MS/MS) assay that has been certified by the Center for Disease Control's HoST Program (19).

Isothermal calorimetry (ITC) experiments were performed using fully automated Auto-ITC200 calorimeter from MicroCal (Northampton, Mass.) provided by Automated Biological calorimetry Facility (Huck Institutes of Life Sciences, University Park, Pa.). SHBG was reconstituted in 30 mM HEPES buffer, pH 7.4, to a final concentration of 5 μM. Testosterone standard was prepared in DMSO and diluted in protein buffer to a concentration of 100 μM in 5% DMSO. DMSO was added to SHBG by weight to match DMSO content in the testosterone solution. Samples were degassed prior to loading to the calorimeter. Testosterone was injected into the protein solution in 32 equal steps. Heat produced by each injection was measured by the calorimeter. Interval between injections was set at 240 seconds so that the temperature could return to baseline. The amount of heat generated after each injection (after subtracting the heat of dilution of ligand in buffer) was integrated to produce calorimetric binding isotherm depicting the relationship of the total heat generated in the reaction to testosterone-to-SHBG molar ratio.

Application of the Novel Algorithm to Clinical Trials Data. Free testosterone concentrations determined using the novel algorithm described herein and by the Vermculen's law-of-mass-action equation, were compared (implemented in a spreadsheet by Mazer) against those measured using the reference method (equilibrium dialysis), in samples derived from randomized testosterone trials in men (101) and women (102). These samples had been collected in fasting state in the morning, stored immediately after collection at −80° C., and never thawed.

Testosterone in Men with Erectile Dysfunction (TED) Trial. The TED Trial, whose design and results have been published (20), was a randomized, placebo-controlled trial to determine whether addition of testosterone to an optimized regimen of sildenafil citrate improves erectile function in men with erectile dysfunction (ED) and low testosterone levels. At baseline and after 12-weeks of testosterone or placebo administration, serum total testosterone concentrations were measured using LC-MS/MS and SHBG concentrations measured by a two-site immunofluorometric assay (DELFIA® SHBG Kit, cat # A070-101. Perkin-Elmer, Waltham. Mass.). Free testosterone concentrations were measured in the same samples by equilibrium dialysis.

### Results

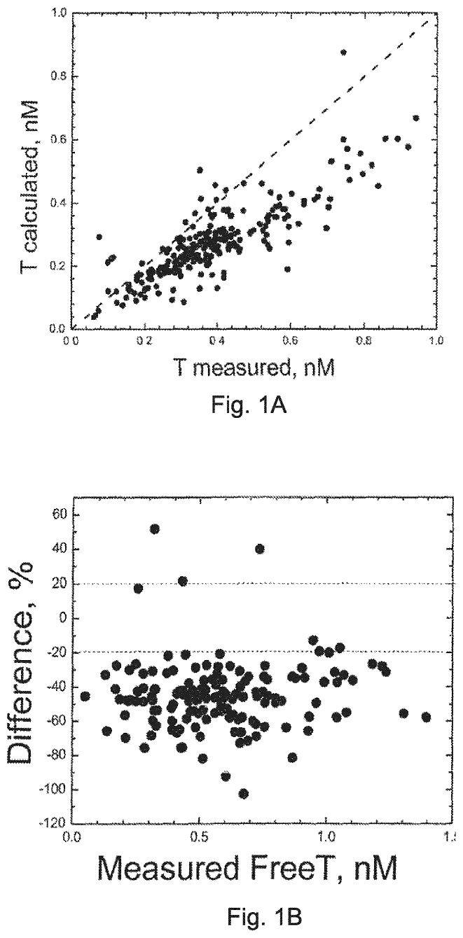

The free testosterone concentrations calculated by the Vermeulen's equation (24) were compared with those measured using equilibrium dialysis in samples derived from participants in the TED Trial. As shown in FIG. 1A, free testosterone concentrations estimated by this equation were significantly lower than those measured by equilibrium dialysis. Bland Altman plot (FIG. 1B) confirmed the substantial underestimation of free testosterone concentration by the Vermeulen's equation relative to that measured by equilibrium dialysis, although the estimation error was not linearly related to the measured free testosterone concentrations. To determine the molecular basis of this discrepancy, we used three experimental approaches to characterize testosterone's binding to SHBG: the binding isotherms, the ligand depletion curve, and the isothermal titration calorimetry (ITC).

Testosterone:SHBG binding Isotherms display evidence of homoallosteric association between testosterone and SHBG dimers. To generate the binding isotherms, 5, 10 or 20 nM SHBG protein (dimer) was incubated with graded concentrations of testosterone (0 to 400 nM) at room temperature (20° C.). The mixture was dialyzed overnight against an equal volume of the dialysis buffer and testosterone concentration in the dialysate was determined by LC-MS/MS. When bound testosterone concentration was plotted against total testosterone concentration (FIG. 2A), the binding isotherm displayed several distinct features: the existence of two distinct saturation plateaus (including an apparent plateau at lower testosterone concentrations), and asymmetry of the isotherm around the EC50 value. The relationship of bound testosterone concentration to total testosterone could not be adequately fit by the Vermeulen's model/equation, which assumes that the two monomers within the SHBG dimer have similar binding constant.

The new Multi-step Dynamic Binding Model with Complex Allostery presented in this study was developed iteratively to encompass all the features of the binding isotherms. In an attempt to comprehensively fit binding isotherms, several models were examined: a) the prevailing equations suggesting a homogenous interaction of two testosterone molecules, b) the monomers within dimer exhibiting distinct affinity constants, c) simple allostery where binding of first ligand alters the affinity of the second site and d) the new Multi-step Dynamic Binding Model with Complex Allostery including two distinct SHBG dimer microstates in equilibrium such that the equilibria between the unliganded and mono-liganded readjusts as the concentration of testosterone is increased. Consistent with the crystal structure data, all the models were constrained to eventually converge on to a single double liganded conformational state of SHBG dimer. Of all the models tested, the new Multi-step Dynamic Binding Model with Complex Allostery adequately fit the binding isotherms. The schematic representation in FIG. 3 summarizes the complex dynamics of the T:SHBG interaction. In this model it was further tested if S1T and S1′T microstates were distinguishable or not. That model with converged S1T and S1′T states again fails to fit the data. The new Multi-step Dynamic Binding Model with Complex Allostery fit the binding isotherm optimally and explained the observed saturation plateaus, including the plateau at lower testosterone concentrations, and the asymmetry of the binding isotherm around the ES50.

Testosterone Depletion Curves. As an independent and complementary assessment of testosterone's binding to SHBG, we incubated various amounts of SHBG (0.1 to 500 nM) with a fixed concentration of testosterone, and analyzed the depletion of unbound testosterone when increasing concentrations of SHBG were added. These depletion curves were generated at several different testosterone concentrations (6 nM, 12 nM, 18 nM, and 32 nM). Mixtures of testosterone and SHBG were dialyzed overnight against a similar volume of the dialysis buffer and free testosterone concentration was measured by LC-MS/MS. The relationship of free testosterone concentration to SHBG concentration in the depletion experiments (FIG. 2B) was again best fit using the new Multi-step Dynamic Binding Model with Complex Allostery. The Vermeulen model did not provide an optimum fit (data not shown).

Isothermal Titration calorimetry (ITC). To validate the new Multi-step Dynamic Binding Model with Complex Allostery further and to evaluate the thermodynamic parameters associated with testosterone binding to SHBG, ITC experiments were performed. The ITC isotherm has a characteristic shoulder (FIG. 2C) and cannot be adequately described as a simple sigmoidal curve predicted by the Vermeulen model nor by models B and C. Using the computational framework developed in LABVIEW (21), fits of the ITC data to the new Multi-step Dynamic Binding Model with Complex Allostery were generated (FIG. 3). Model constants obtained as a result of the linked fit in FIGS. 2A-2B were used as a starting point for the fit. The new Multi-step Dynamic Binding Model with Complex Allostery model provided an excellent fit for the experimental data derived from the ITC. The shape of the ITC curve can be explained as a convoluted result of testosterone binding and multiple conformational rearrangements defined by the new Multi-step Dynamic Binding Model with Complex Allostery. While, the independent enthalpy parameters for each of the individual reactions comprising the model were used, they are not simultaneously identifiable.

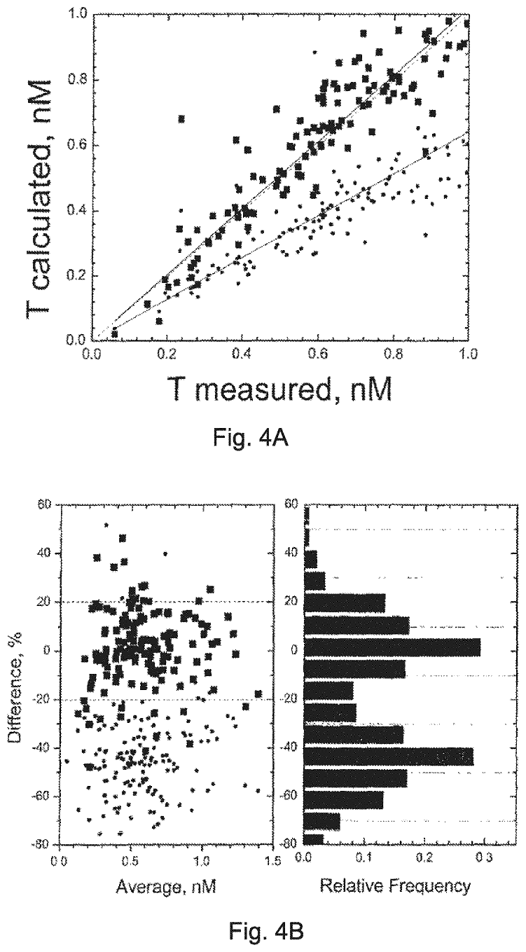

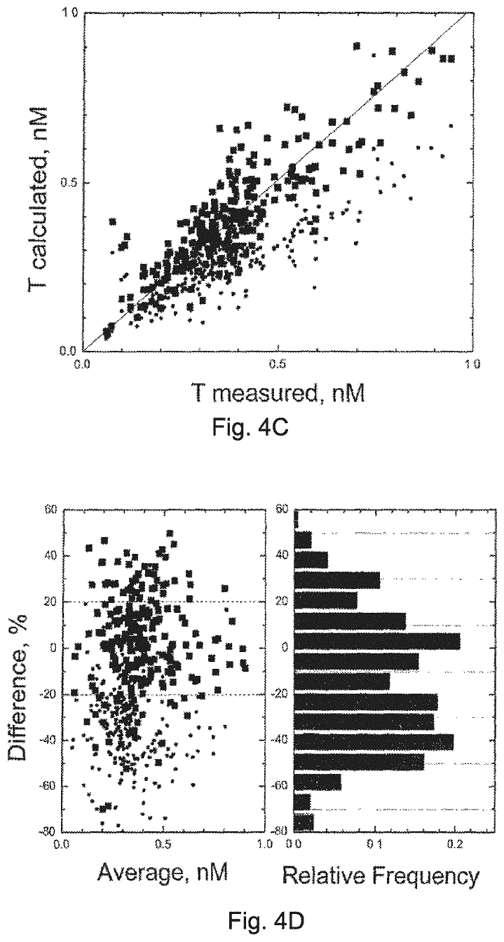

Application of the New Algorithm to Clinical Trials Data. The new model was applied to data generated in the TED trial (FIGS. 4A-4D). The free testosterone concentrations were calculated by the prevailing equations (23-24) and those calculated using the new algorithm based on new Multi-step Dynamic Binding Model with Complex Allostery (FIGS. 4A-4D). The prevailing model (17) significantly underestimated free testosterone levels relative to equilibrium dialysis in men participating in the TED trial. In contrast, the new Multi-step Dynamic Binding Model with Complex Allostery model provided values that were not statistically different from those measured by equilibrium dialysis (slope 1.01±0.01) in both men and women. The Bland-Altman plots (FIGS. 4A-4D) show the absence of any systematic difference between the values derived from the new Multi-step Dynamic Binding Model with Complex Allostery model and those obtained using the equilibrium dialysis in either men or women; the relative deviation of the values calculated using the new Multi-step Dynamic Binding Model with Complex Allostery model from those measured using equilibrium dialysis was evenly distributed around 0, likely reflecting multiple sources of measurement error in the testosterone assay, SHBG assay, and in the equilibrium dialysis method (FIGS. 4A-4D).

### Discussion

It is demonstrated herein that the current model of testosterone binding to SHBG that has formed the basis of the law-of-mass-action equations for several decades to estimate free testosterone concentrations is erroneous. While the discrepancy between testosterone concentrations estimated using the available prevailing equations and those measured using the equilibrium dialysis method has been recognized ( ), the present data provide a rational mechanistic explanation for this discrepancy that had remained obscure previously. The experimental data from the binding isotherms, the SHBG depletion curves, and the ITC cannot be explained by the existing SHBG-T interaction model of single binding site or two identical, non-interacting binding sites on SHBG. The experimental data are poorly explained even by simple models for homotropic allostery within a dimer (e.g., Koshland-Nemesy-Filmer (22) and Monod-Wynan-Changeaux (23) models. In contrast, the new Multi-step Dynamic Binding Model with Complex Allostery (24, 25) optimally fits the data from the binding isotherms and also explains adequately the depletion curves and the ITC data. Furthermore, in samples derived from male and female participants in two separate clinical trials, testosterone concentrations calculated using the new Multi-step Dynamic Binding Model with Complex Allostery were statistically not different from those measured by the equilibrium dialysis.

The proposed new Multi-step Dynamic Binding Model with Complex Allostery indicates that in the absence of testosterone, SHBG molecule can assume one of at least two inter-converting microstates in a dynamic equilibrium. The binding of testosterone to one of the monomers of the SHBG dimer in a given microstate affects the interaction of testosterone with the unoccupied second binding site on the SHBG dimer. The model suggests a dynamic readjustment of populations of intermediate species as testosterone concentrations are altered. Independent experimental validation of model was performed by generating ligand depletion and ITC studies. The data from three sets (equilibrium binding, ligand depletion and ITC) were successfully fit by the new Multi-step Dynamic Binding Model with Complex Allostery. Without wishing to be bound by theory, the parameters in the model are not uniquely determined and could require detailed experimental evaluation of each microstate in multiple equilibria.

Prevailing hypothesis that has formulated the basis of the equations/expressions by Vermeulen assumes that monomers within a dimer display identical association constant without any dynamic interaction between the subunits. The general idea was supported by ligand bound crystal structure of SHBG (36). Parenthetically, the crystal structures are typically obtained in saturating concentrations of ligands. It is possible that the inability to resolve the unliganded SHBG structure (26) as well as the increased stability of SHBG upon ligand binding (27) may be related to the significant rearrangement of SHBG molecule upon binding of the first ligand, as predicted by the new Multi-step Dynamic Binding Model with Complex Allostery (24). The additional energy barrier that SHBG has to overcome may result in altered affinity for binding of the second ligand molecule. Further studies utilizing dimerization deficient mutants as well as molecular dynamics simulations are required to precisely define biophysical parameters associated with each ligand binding event.

The algorithm based on new Multi-step Dynamic Binding Model with Complex Allostery was applied to human samples obtained from randomized trials in men and women. It was found that over a wide range of testosterone concentrations prevalent in the male and female participants in the two testosterone trials, the free testosterone concentrations determined using the new algorithm were similar to those measured using the equilibrium dialysis. Also, the algorithm was applicable over a wide range of testosterone and estradiol concentrations in men and women. Without wishing to be bound by theory, there is a possibility that very high estrogen concentrations, such as those that may be observed during pregnancy, may affect testosterone binding to SHBG, introducing potential error in the calculated concentrations.

The current algorithm and the experimental data reported here were generated using wild type SHBG which is present in nearly 98% of Caucasians. Genome wide association studies have revealed several SHBG polymorphisms, two of which have been reported to affect testosterone binding to SHBG (28). Therefore, in future, the algorithms may include a term for the SHBG genotype. In summary, the experimental data of testosterone's association with SHBG implicate a complex allostery mechanism within the SHBG dimer. The new Multi-step Dynamic Binding Model with Complex Allostery provides excellent fit to the experimental data generated using three different techniques. Unlike the existing equations based on homogenous binding of testosterone with SHBG, which reveal systematic discrepancy from the values obtained by equilibrium dialysis, the free testosterone concentrations derived using the new model do not differ significantly from those measured using equilibrium dialysis.

### REFERENCES

1. Vermeulen A. Verdonek L & Kaufman J M (1999) A critical evaluation of simple methods for the estimation of free testosterone in serum. J Clin Endocrinol Metab 84(10): 3666-3672.
2. Rosner W, Auchus R J, Azziz R, Sluss P M & Raff H (2007) Position statement: Utility, limitations, and pitfalls in measuring testosterone: An endocrine society position statement. J Clin Endocrinol Metab 92(2): 405-413.
3. Rosner W (1997) Errors in the measurement of plasma free testosterone. J Clin Endocrinol Metab 82(6): 2014-2015.
4. Winters S J, Kelley D E & Goodpaster B (1998) The analog free testosterone assay: Are the results in men clinically usefill?. Clin Chem 44(10): 2178-2182.
5. Sinha-Hikim I, et al (1998) The use of a sensitive equilibrium dialysis method for the measurement of free testosterone levels in healthy, cycling women and in human immunodeficiency virus-infected women. J Clin Endocrinol Metab 83(4): 1312-1318.
6. Van Uytfanghe K, et al (2004) Evaluation of a candidate reference measurement procedure for serum free testosterone based on ultrafiltration and isotope dilution-gas chromatography-mass spectrometry. Clin Chem 50(11): 2101-2110.
7. Adachi K, et al (1991) Measurement of plasma free steroids by direct radioimmunoassay of ultrafiltrate in association with the monitoring of free components with [14C]glucose. Clin Chin Acta 200(1): 13-22.
8. Morley J E, Patrick P & Perry H M. 3rd (2002) Evaluation of assays available to measure free testosterone. Metabolism 51(5): 554-559.
9. Morales A, Collier C P & Clark A F (2012) A critical appraisal of accuracy and cost of laboratory methodologies for the diagnosis of hypogonadism: The role of free testosterone assays. Can J Urol 19(3): 6314-6318.
10. Rosner W (1991) Plasma steroid-binding proteins. Endocrinol Metab Clin North Am 20(4): 697-720.
11. Rosner W (2006) Sex steroids and the free hormone hypothesis. Cell 124(3): 455-6; author reply 456-7.
12. Mendel C M (1989) The free hormone hypothesis: A physiologically based mathematical model. Endocr Rev 10(3): 232-274.
13. Sodergard R, Backstrom T, Shanbhag V & Carstensen H (1982) Calculation of free and bound fractions of testosterone and estradiol-17 beta to human plasma proteins at body temperature. J Steroid Biochem 16(6): 801-810.
14. Nanjee M N & Wheeler M J (1985) Plasma free testosterone—is an index sufficient?. Ann Clin Biochem 22 (Pt 4)(Pt 4): 387-390.
15. Ly L P & Handelsman D J (2005) Empirical estimation of free testosterone from testosterone and sex hormone-binding globulin immunoassays. Eur J Endocrinol 152(3): 471-478.
16. Sartorius G, Ly L P, Sikaris K, McLachlan R & Handelsman D J (2009) Predictive accuracy and sources of variability in calculated free testosterone estimates. Ann Clin Biochem 46(Pt 2): 137-143.
17. Vermeulen A, Stoica T & Verdonek L (1971) The apparent free testosterone concentration, an index of androgenicity. J. Clin Endocrinol Metab 33(5): 759-767.
18. Ly L P, et al (2010) Accuracy of calculated free testosterone formulae in men. Clin Endocrinol (OxJ) 73(3): 382-388.
19. Bhasin S, et al (2011) Reference ranges for testosterone in men generated using liquid chromatography tandem mass spectrometry in a community-based sample of healthy nonobese young men in the framingham heart study and applied to three geographically distinct cohorts. J Clin Endocrinol Metab 96(8): 2430-2439.
20. Spitzer M, et al (2012) Effect of testosterone replacement on response to sildenafil citrate in men with erectile dysfunction: A parallel, randomized trial. Ann Intern Med 157(10): 681-691.
21. Zakharov M N, Bhasin S, Szafran A T & Mancini M A, Jasuja R. (2011 (in press)) Numerical framework to model temporally-resolved multi-stage dynamic systems. COMPUT METH PROG BIO
22. Koshland D E, Jr. Nemethy G & Filmer D (1966) Comparison of experimental binding data and theoretical models in proteins containing subunits. Biochemistry 5(1): 365-385.
23. MONOD J, WYMAN J & CHANGEUX J P (1965) On the nature of allosteric transitions: A plausible model. J Mol Biol 12: 88-118.
24. Freiburger L A, et al (2011) Competing allosteric mechanisms modulate substrate binding in a dimeric enzyme. Nat Struct Mol Biol 18(3): 288-294.
25. Hilser V J & Thompson E B (2007) Intrinsic disorder as a mechanism to optimize allosteric coupling in proteins. Proc Nal Acad Sci USA 104(20): 8311-8315.
26. Avvakumov G V, Cherkasov A, Muller Y A & Hammond G L (2010) Structural analyses of sex hormone-binding globulin reveal novel ligands and function. Mol Cell Endocrinol 316(1): 13-23.
27. Awakumov G V, Muller Y A & Hammond G L (2000) Steroid-binding specificity of human sex hormone-binding globulin is influenced by occupancy of a zinc-binding site. J Biol Chem 275(34): 25920-25925.
28. Ohlsson C, et al (2011) Genetic determinants of serum testosterone concentrations in men. PLoS Genet 7(10): e1002313.

### Example 2: A New Multi-Step Dynamic Binding Model with Complex Allostery of Testosterone's Binding to Sex Hormone Binding Globulin

Circulating free testosterone (FT) levels have been used widely in the diagnosis and treatment of hypogonadism in men. Due to experimental complexities in FT measurements, the Endocrine Society expert panel has recommended the use of calculated FT (cFT) as an appropriate approach for estimating FT. It is demonstrated herein that the prevailing model of testosterone's binding to SHBG, which assumes that each SHBG dimer binds two testosterone molecules and that the two binding sites on SHBG have similar binding affinity, provides values of free testosterone that differ substantially from those obtained using equilibrium dialysis.

Described herein is the characterization of testosterone's binding to SHBG using equilibrium dialysis (binding isotherms varying both ligand and protein) and isothermal titration calorimetry. These experimental data were utilized in the development of a new model of testosterone's binding to SHBG; and this new model permitted the determination of free testosterone concentrations and comparison of these values to those derived from equilibrium dialysis.

Experimental data from equilibrium dialysis experiments, and isothermal titration calorimetry provide evidence of complex homoallostery within SHBG. Described herein is a New Multi-Step Dynamic Binding Model with Complex Allostery encompassing at least two inter-converting microstates in unliganded SHBG, readjustment of equilibria between unliganded states upon binding of the first ligand molecule, and allosteric interaction between two binding sites of SHBG dimer. Free testosterone concentrations determined using framework incorporating intra-dimer allostery did not differ from those measured using equilibrium dialysis in samples from clinical trials Testosterone's binding to SHBG is demonstrated herein to be a multi-step process that involves complex homo-allostery within SHBG dimer, cFT values obtained using the new model have close correspondence with those measured using equilibrium dialysis.

### Introduction

Testosterone, the major androgen in humans, circulates in blood bound largely to sex hormone binding globulin (SHBG) and albumin (Rosner 1991, Hammond and Bocchinfuso 1996, Bhasin et al 2010. Mendel 1989, Rosner et al 2007). Testosterone can also bind to orosomucoid and transcortin proteins, but the amount of testosterone bound to these proteins in human plasma is negligible. According to the free hormone hypothesis, only the unbound or free fraction can cross the plasma membrane and is biologically active (Rosner 1991, Hammond and Bocchinfuso 1996, Bhasin et al 2010, Mendel 1989, Rosner et al 2007). In many conditions that affect SHBG concentrations, such as obesity, diabetes, aging, hyperthyroidism, liver disease, and HIV-infection, total testosterone concentrations are altered because of changes in SHBG concentrations; in these conditions, expert panels have recommended determination of free testosterone (FT) concentration to obtain an accurate assessment of androgen status (Rosner 1991, Hammond and Bocchinfuso 1996, Bhasin et al 2010, Mendel 1989, Rosner et al 2007).

The current model of testosterone's binding to SHBG assumes that each SHBG dimer binds two testosterone molecules, and that each of the two binding sites on SHBG dimer has similar binding affinity. Equations to determine FT were proposed by Vermeulen and others (Rosner et al 2007, Sodergard et al 1982, Vermeulen et al 1971, Mazer 2009)(Sodergard et al 1982, Vermeulen et al 1971). We show here that the prevailing model of testosterone's binding to SHBG is erroneous. The data from equilibrium dialysis and isothermal titration calorimetry (ITC) experiments provide evidence for ligand modulated allosteric interaction between the binding sites on the two SHBG monomers.

Reflecting the growing interest in men's health and the success of pharmaceutical advertising, testosterone sales have grown from 23 million dollars in 1993 to 70 million in 2000 to 1.7 billion dollars in 2012 (Spitzer et al 2012). Testosterone is the second most frequently ordered test, next only to 25-hydroxyvitamin D. In 2012, nearly 4 million free testosterone tests were performed in the USA alone. A number of direct and indirect methods—equilibrium dialysis, ultrafiltration, tracer analog methods, and calculations based on homogenous (equal affinity of testosterone for each monomer in SHBG dimer) binding—have been developed for the determination of FT levels (Rosner et al 2007, Sodergard et al 1982. Vermeulen et al 1971, Mazer 2009, Rosner 1997, Winters et al 1998, Vermeulen et al 1999. Sinha-Hikim et al 1998, Van Uytfanghe et al 2004, Adachi et al 1991, Morley et al 2002)(Sodergard et al 1982, Vermeulen et al 1971). Expert panels have expressed concern about the accuracy and methodological complexity of the available assays for FT (Rosner et al 2007, Sodergard et al 1982, Vermeulen et al 1971). Recognizing these methodological difficulties in the measurement of free testosterone, the Endocrine Society's Expert Panel suggested that “the calculation of free testosterone from reliably measured total testosterone and SHBG using mass action equations provides the best approach for the estimation of free testosterone . . . ” (Rosner et al 2007). Therefore, algorithms for calculating FT from total testosterone, SHBG and albumin concentrations have been used widely (Rosner et al 2007, Mazer 2009, Morley et al 2002, Morales et al 2012, Ly et al 2010, Sartorius et al 2009, Bhasin et al 2011, Ly and Handelsman 2005). The equations are either based on the binding mechanism and law of mass-action (Sodergard et al 1982, Mazer 2009, Vermeulen et al 1999) (Sodergard et al 1982, Vermeulen et al 1999, Sartorius et al 2009, Ly and Handelsman 2005, Nanjee and Wheeler 1985) or are empirically-derived (Ly et al 2010, Sartorius et al 2009, Ly and Handelsman 2005, Nanjee and Wheeler 1985) (Sodergard et al 1982, Sartorius et al 2009, Ly and Handelsman 2005, Nanjee and Wheeler 1985).

Described herein is equilibrium dialysis (varying both SHBG and ligand concentrations) and isothermal titration calorimetry (ITC) to characterize testosterone's binding to SHBG. Various possible mechanistic models of molecular interactions, including linear homogeneous binding of testosterone to SHBG as envisioned by Vermeulen (Vermeulen et al 1999), Sodergard (Sodergard et al 1982) and Mazer (Mazer 2009), and various allosteric mechanisms, including allostery with positive and negative cooperativity (Koshland et al 1966. MONOD et al 1965), and an ensemble allosteric mechanism (Hilser and Thompson 2007) were considered. Based on our analyses of the experimental data of testosterone's binding to SHBG, a novel algorithm was constructed for the calculation of FT, which included intra-dimer allostery, which provided the best fit to the totality of experimental data. This new model was applied to determine free testosterone concentrations in samples derived from randomized testosterone trials, the results compared with those obtained using equilibrium dialysis.

### Materials and Methods

Biophysical characterization. Human SHBG purified from serum (Binding Site Group, Birmingham, UK) was characterized by protein gel denaturation-renaturation experiments and by measuring its ability to bind testosterone. Testosterone concentration in the SHBG stock solution, measured using LC-MS/MS, was undetectable. Testosterone standard 1.0 mg/mL.+−0.2% (3.47 mM) was obtained from Cerilliant (Round Rock, Tex.).

Binding profiles were established by the equilibrium dialysis (varying either ligand or protein concentration) were performed in 96-well dialysis plates containing dialysis chambers separated by membranes with 10 kDa cut-off (Harvard Apparatus, Holliston, Mass.). SHBG and testosterone were reconstituted in dialysis buffer (30 mM HEPES pH7.4, 90 mM NaCl, 1 mM MgSO4, 187 μM CaCl₂)), and mixed to a desired concentration. 200 μl of SHBG-testosterone mixture was loaded on one side of the dialysis membrane and dialyzed overnight against equal volume of dialysis buffer (200 μl). The equilibrium was achieved by rotating the dialysis plate overnight at 22° C. 16 hours were determined necessary to achieve an equilibrium. Each concentration/condition was tested in 3 different wells, and each titration was repeated at least 2 times.

Testosterone concentration was measured using liquid chromatography tandem mass spectrometry (LC-MS/MS) assay that has been certified by the Center for Disease Control and has a sensitivity of 2 ng/dL (Bhasin et al 2011).

Isothermal calorimetry (ITC) was performed using automated Auto-ITC200 calorimeter (MicroCal, Northampton, Mass.) at Biological calorimetry Facility (Huck Institutes of Life Sciences, University Park, Pa.). SHBG was reconstituted in 30 mM HEPES buffer, pH7.4, to a final concentration of 5 μM. Testosterone standard was prepared in DMSO and diluted in protein buffer to 100 μM in 5% DMSO. DMSO was added to SHBG by weight to match DMSO content in testosterone solution. Samples were degassed prior to loading to the calorimeter. Testosterone was injected into protein solution in 15 equal steps 2 μl each. Total reaction volume was 203 μl. Isothermal titration calorimetry experiment was repeated twice. Heat produced by each injection was measured by the calorimeter. Interval between injections was set at 240 seconds so that the temperature could return to baseline. The heat generated after each injection (after subtracting the heat of dilution of ligand in buffer) was integrated to produce calorimetric isotherm depicting the relation of the total heat generated in the reaction to testosterone-to-SHBG molar ratio.

Numerical Simulations of Allostery

Various molecular models of testosterone's binding to SHBG were numerically tested using LabVIEW™ (National Instruments, Austin, Tex.) toolkit (Zakharov et al 2012) (available on the world wide web at code.google.com/p/labview-biochemical-framework/). Parameter estimation for the models was performed as described previously (Zakharov et al 2012). Numerical correction for the equilibrium dialysis was incorporated as a part of every simulation model. Since some of the models and equations (Sodergard et al 1982, Vermeulen et al 1999, Nanjee and Wheeler 1985) were developed essentially before the confirmation of the 2 binding sites per SHBG dimer (Avvakumov et al 2001) we adjusted SHBG concentration by the factor of 2 for these models.

The fits of both types of binding profiles and ITC to various models were compared by calculating the residuals and χ² values for each model. The fits to the model incorporating complex allostery consistently gave the smallest χ² value and residuals.

Assessment of FT Concentrations in Clinical Trials. FT determined using the dynamic model developed in this study (cFTZBJ) and Vermeulen's equation (cFTV) (as implemented by Mazer, (Mazer 2009)) were compared with those measured using equilibrium dialysis in samples derived from randomized testosterone trials in men (Spitzer et al 2012) and women (Huang et al 2012). These samples had been collected in fasting state in the morning, stored at −80° C., and never thawed.

Testosterone in Men with Erectile Dysfunction (TED) Trial, whose results have been published (Spitzer et al 2012)(Spitzer et al 2012), was a randomized trial to determine whether addition of testosterone to an optimized regimen of sildenafil citrate is superior to placebo in improving erectile function in men with erectile dysfunction (ED) and low testosterone. At baseline and after 12-weeks of testosterone or placebo administration, total testosterone concentrations were measured using LC-MS/MS and SHBG concentrations using a two-site immunofluorometric assay (DELFIA®, Perkin-Elmer, Waltham, Mass.) (21). FT was measured in the same samples by equilibrium dialysis (Bhasin et al 2012).

### Statistical Analysis.

The model fits of experimental data were assessed using chi-square statistics. For clinical trials data, the distributions of measured and calculated FT were derived for each of the relevant samples. Agreement between measured and calculated FT values was estimated using Deming (orthogonal) Regression, and Bland-Altman style plots were used to assess the difference between calculated and measured concentrations as a function of the measured concentration. Graphical depictions of association between FT, total testosterone, and SHBG were generated, with scatter plot smoothing using Generalized Additive Models with tensor product smooths (Wood 2006).

### Results

Preliminary studies revealed that cFT values obtained using the Vermeulen's equation in samples derived from the TED Trial were significantly lower than those measured by equilibrium dialysis. To determine the molecular basis of this discrepancy, three experimental approaches were used to characterize testosterone's binding to SHBG: binding isotherms, ligand depletion curves, and isothermal titration calorimetry (ITC). The overview of different molecular models is presented below. Each of this models assume 2 binding sites per SHBG dimer.

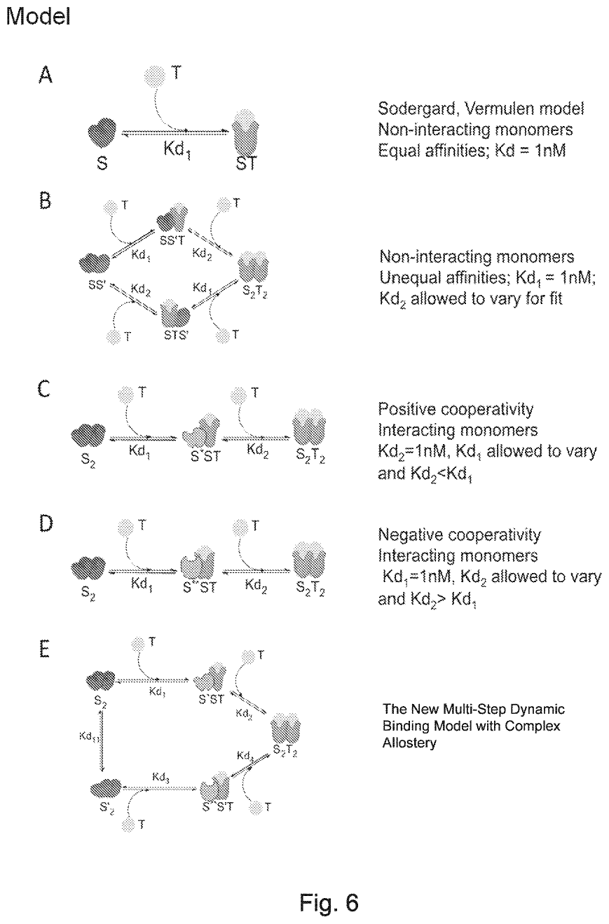

The simplest of the SHBG T interaction models is Vermeulens model, assuming that each binding site interacts with T with the same affinity, regardless of the other binding site occupancy (FIG. 6, model A). The monomers are not interacting, therefore only one subunit is depicted. Second model is a model, when non-interacting monomers are allowed to have different affinities (FIG. 6, model B). Third and fourth models are models of positive and negative cooperativity as postulated by (Koshland et al 1966). Binding of the first testosterone molecule either facilitates (FIG. 6, model C) or suppresses (FIG. 6, model D) binding of the second molecule (symmetric reactions are not listed for clarity). The sign of the cooperativity is modelled by the relation of the first equilibrium binding constant (Kd1) to the second one (Kd2). Kd1>Kd2 means positive cooperativity, Kd1<Kd2 means negative cooperativity. Model in FIG. 6 model E, the new Multi-step Dynamic Binding Model with Complex Allostery. The equilibrium between those states while unbound is governed by a unimolecular equilibrium constant Kd1 1. Upon binding of the first testosterone molecule (with equilibrium constants Kd1 and Kd2) SHBG dimer assumes two different states, each of them with different affinity for the second T molecule (Kd2 and Kd4). Consistent with the reported crystal structure of liganded SHBG (Grishkovskaya et al 2000, Grishkovskaya et al 1999, Grishkovskaya et al 2002, Avvakumov et al 2002), all models were constrained to eventually converge to a single double-liganded conformational state of SHBG dimer. These models were examined and the model with the best fit of the experimental data was determined.

Biophysical characterization of testosterone's binding to SHBG reveals evidence of complex homo-allostery within SHBG dimer.

Equilibrium Dialysis: Binding Isotherms

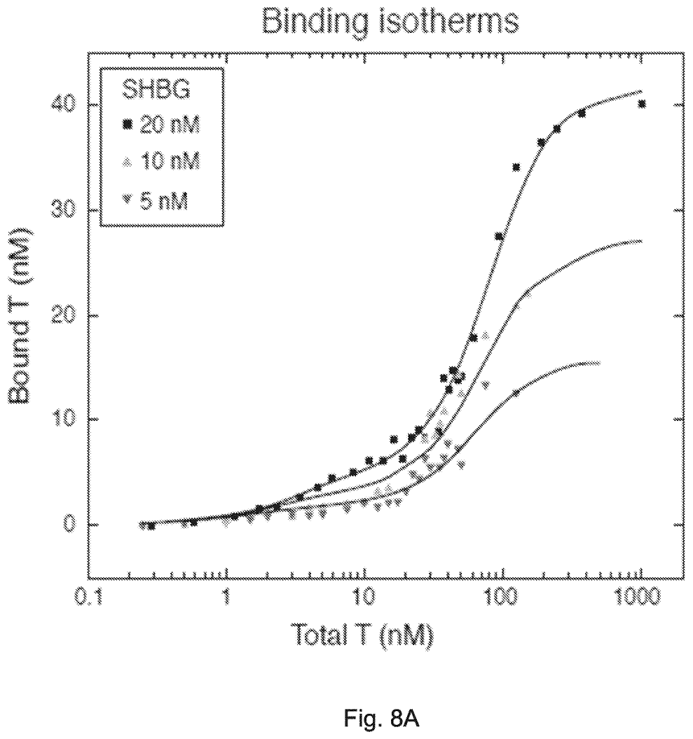

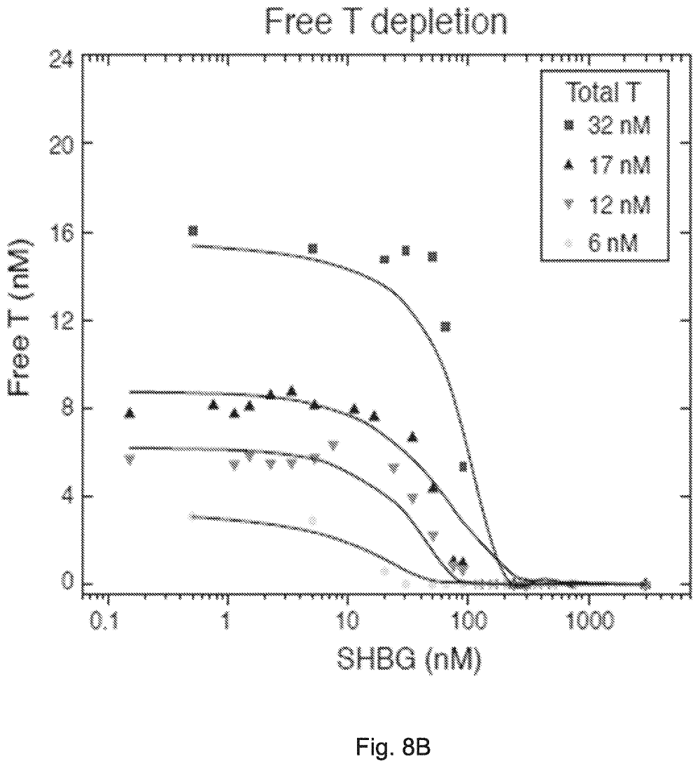

To generate the binding isotherms, 5, 10 or 20 nM SHBG (dimer) was incubated with graded concentrations of testosterone (0 to 400 nM) at 22° C., as described in the methods section. When bound testosterone concentration was plotted against total testosterone concentration (FIG. 5A, FIG. 8A), the binding isotherm displayed several characteristic features: two distinct saturation plateaus (including an apparent plateau at lower testosterone concentrations), asymmetry of the isotherm around the EC50 value. The relation of bound testosterone to total testosterone could not be adequately explained by Vermeulen's model. Only the new model eliciting intra-dimer allostery fit the binding isotherm optimally with the lowest χ² (FIG. 8A) and explained the observed saturation plateaus, including the plateau at lower testosterone concentrations, and the asymmetry of binding isotherm around EC50. FIG. 5A presents the fit to the new Multi-step Dynamic Binding Model with Complex Allostery. Fits to other models of FIG. 6 as well as the analysis of the residuals is presented in supplementary material (FIG. 8A-8B). Additionally, it was tested if S*ST and S**S′T microstates were distinguishable. It was found that model with converged S*ST and S**S′T states failed to fit the data.

Equilibrium Dialysis: Testosterone Depletion Curves

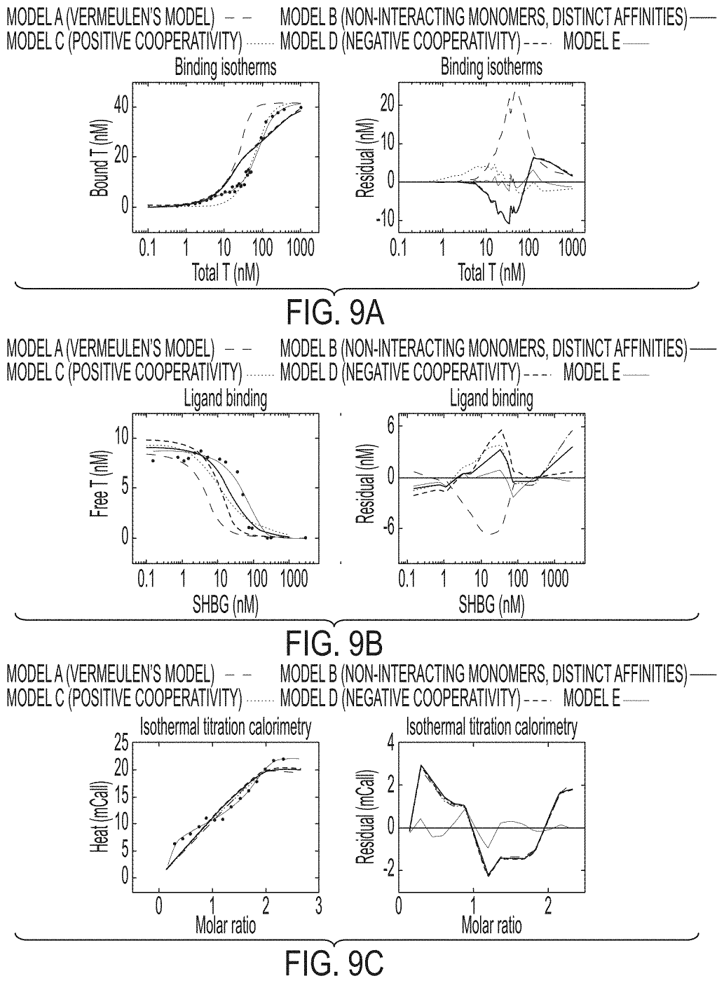

As an independent assessment of testosterone's binding to SHBG, various amounts of SHBG (0.1 to 0.5 μM) were incubated with a fixed concentration of testosterone, and the depletion of unbound testosterone when increasing concentrations of SHBG were added was analyzed (FIG. 5B, FIGS. 9A-9C). These depletion curves were generated at various testosterone concentrations (3, 6, 8.7, and 16 nM). Sample preparation and measurement procedure are described in the Methods section. The relation of FT to SHBG concentration in depletion experiments was again best fit using the model that included complex allostery. The analysis of residuals (FIG. 8B) revealed that the optimal fit once again provided by the new Multi-step Dynamic Binding Model with Complex Allostery model.

Isothermal Titration Calorimetry (ITC):

To validate the new model further and to evaluate the thermodynamic parameters associated with testosterone's binding to SHBG, the heat produced as progressively larger amounts of testosterone bind to SHBG, were measured using the ITC. The ITC data has a characteristic shoulder (FIG. 5C) and cannot be described as a simple sigmoidal curve predicted by Vermeulen's model (FIG. 8A-8B). Using the computational framework developed in LabVIEW (Zakharov et al 2012), we generated the fits of the ITC data (For mathematical treatment, see supplementary material S1, which follows (Freiburger et al 2011)). The shape of ITC curve can be explained as a convoluted result of testosterone's binding and multiple conformational rearrangements defined by the comprehensive model incorporating allostery. Model constants obtained as a result of linked fit in FIGS. 5A and 5B were used as a starting point for the fit; enthalpies and reaction constants computed from the fit ITC data are presented in FIG. 7. While we used an independent enthalpy parameter for each reaction in the model, they are not simultaneously identifiable.

Effects of Estradiol and Dihydrotestosterone (DHT). Addition of estradiol 170 in concentrations ranging from 10 to 500 pg/mL had no significant effect on percent free testosterone. Similarly, free testosterone concentrations in men treated with graded doses of testosterone enanthate plus placebo whose DHT concentrations extended from physiologic to supraphysiologic range did not differ from those treated with testosterone enanthate plus dutasteride whose DHT concentrations were very low (Bhasin et al 2012), indicating that DHT over the range of concentrations relevant in male and female physiology has little effect on percent free testosterone.

Application of new Multi-step Dynamic Binding Model with Complex Allostery to Clinical Trials Data. SHBG and albumin are predominantly the two proteins that bind testosterone with significant affinity; the binding affinities of transcortin and orosomucoid for testosterone are extremely low. Accordingly, we included testosterone's interaction with albumin along with complex allostery in equilibria describing its binding to SHBG to determine FT (FTZBJ) in serum samples from the Testosterone in Erectile Dysfunction (TED) Trial ((Spitzer et al 2012). The comprehensive model was implemented in the LabVIEW framework (Zakharov et al 2012) (data not shown).

cFTV significantly underestimated FT levels relative to equilibrium dialysis in men participating in the TED trial. In contrast, cFTZBJ provided values that were not statistically different from those measured by equilibrium dialysis in men (slope 1.01±0.01). The Bland-Altman plots (data not shown) found no significant difference between the cFTZBJ and those obtained using equilibrium dialysis in either men or women; the relative deviation of values calculated using the new model from those measured using equilibrium dialysis was evenly distributed around 0, likely reflecting multiple sources of measurement error in testosterone assay, SHBG assay, and equilibrium dialysis. The Deming regression was used to compare the values derived using the new model and cFTv with those obtained using equilibrium dialysis, which reaffirm the substantial bias of FTv from values derived using equilibrium dialysis and the substantially better correspondence between cFTZBJ and equilibrium dialysis.

Relation between Percent FT with Total Testosterone and SHBG. Intra-dimer complex allostery suggests that SHBG can regulate FT fraction over a wide range of total testosterone concentrations without getting saturated. Indeed, it was found that percent FT calculated using the new model changed very modestly over a wide range of total testosterone concentrations. In contrast, the Vermeulen's equation suggests a negative relation between percent FT and total testosterone. Furthermore, as SHBG concentrations increase, percent FT calculated using our new model shows only a modest decline in contrast to the marked decline in percent FT calculated using Vermeulen's equation.

### Discussion

Several lines of evidence presented here indicate that the existing model of testosterone's binding to SHBG (single binding site or two identical, non-interacting binding sites on SHBG) that has formed the basis of Vermeulen, Sodergard, and Mazer's (Sodergard et al 1982, Vermeulen et al 1971, Mazer 2009, Vermeulen et al 1999) equations to estimate free testosterone concentrations does not accurately explain the experimental data from equilibrium dialysis and ITC (even if corrected for 2 binding sites per dimer stoichiometry). While the discrepancy between testosterone concentrations estimated using the above-mentioned equations and those measured using equilibrium dialysis has been recognized (Ly et al 2010), the data presented herein provide a mechanistic explanation for this discrepancy. Simple models of homotropic allostery with positive or negative cooperativity within a dimer also did not adequately explain the experimental data. Only the dynamic model that incorporates complex allostery optimally fits the experimental data derived from three independent methods. Furthermore, FT concentrations calculated using the new model incorporating complex allostery were not significantly different from those measured by equilibrium dialysis in samples derived from men and women in two separate clinical trials. The analysis of the steady state experimental binding data presented herein indicate that in the absence of testosterone, SHBG molecule can assume one of at least two inter-converting microstates in dynamic equilibrium. The binding of testosterone to one of the monomers of the SHBG dimer in a given microstate affects the interaction of testosterone with the unoccupied second binding site on the SHBG dimer. The model suggests a dynamic re-adjustment of populations of intermediate species as testosterone concentration is changing. Because of the dynamic nature of these processes, all parameters of the model cannot be uniquely determined. Thus, testosterone's binding to SHBG is not a single linear reaction but rather a series of inter-related molecular processes that can be described by the new Multi-step Dynamic Binding Model with Complex Allostery shown in FIG. 7. The fits of the data to the new model that incorporates complex allostery display a dynamic re-adjustment of populations of intermediate species as testosterone concentration changes. Because of the multiple equilibria and dynamic and allosteric nature of these processes, testosterone's binding to SHBG cannot be described as a simple linear equation of ligand binding equilibrium. Accordingly, the multi-species allostery model was implemented in Lab VIEW framework (Zakharov et al 2012). Optimal fit parameters for the ITC and equilibrium dialysis data (sections 3.1.1, 3.1.2, 3.1.3) were obtained by the Levenberg-Marquardt optimization, using globalfit approach similar to (Freiburger et al 2011). This set of parameters that can be used to compute free testosterone are shown in FIGS. 7 and 6, model E.

The new dynamic model leads to the reconsideration of several dogmas related to testosterone's binding to SHBG and has important physiologic and clinical implications. First, the fraction of circulating testosterone which is free is substantially greater (2.9±0.4%) than has been generally assumed (% cFTV 1.5±0.4%). Second, percent FT is not significantly related to total testosterone over a wide range of total testosterone concentrations. However, the percent FT declines as SHBG concentrations increase, although it does not decline as precipitously as predicted by the Vermculen's model. Due to the allostery between the two binding sites, SHBG is able to regulate FT levels in much larger dynamic range.

Several factors may have contributed to the formulation of the prevailing hypothesis that monomers within SHBG dimer display identical binding affinity without any dynamic interaction between the monomers. The extant ligand binding equations were formulated in an era that preceded the appreciation of the dimeric nature of circulating SHBG. However, the Mazer's implementation (Mazer 2009) of the Vermeulen's model, as applied in these analyses used the correct stoichiometry—two molecules of testosterone binding to each SHBG dimer. Therefore, the discrepancy between cFTv and the reference method cannot be explained solely on the basis of incorrect stoichiometry. Furthermore, the range of testosterone and SHBG concentrations used in binding experiments and Scatchard plots were limited and did not generally extend into the high range (Metzger et al 2003, Dunn et al 1981, Hauptmann et al 2003, Petra et al 1986), which may have prevented appreciation of the second binding site. Also, a single crystal structure of the ligand-bound SHBG may have further contributed to the erroneous impression that the binding events associated with testosterone's binding to two binding sites on SHBG dimer are identical. The inability to resolve the unliganded SHBG structure (Avvakumov et al 2000, Avvakumov et al 2010)(Avvakumov et al 2010) as well as the increased stability of SHBG upon ligand binding (Avvakumov et al 2000)(Avvakumov et al 2000) may be related to significant rearrangement of SHBG molecule upon binding of the first ligand, as predicted by the conformational heterogeneity in complex allostery. The additional energy barrier that SHBG has to overcome may result in altered affinity for binding of the second ligand molecule.

While the new algorithm developed in this study accurately determines FT, effects of other interacting hormones mandates further investigation. In a previous study (Bhasin et al 2012), it was found that free testosterone concentrations in men treated with graded doses of testosterone enanthate plus placebo whose DHT concentrations extended from physiologic to supraphysiologic range did not differ from those treated with testosterone enanthate plus dutasteride whose DHT concentrations were very low, indicating that DHT over the range of concentrations relevant in in male and female physiology has little effect on percent free testosterone. Similarly, over a wide range of estradiol concentrations prevalent in men and women, cFTZBJ concentrations were similar to those measured using equilibrium dialysis. Without wishing to be bound by theory, very high estrogen concentrations, such as those observed during pregnancy, or very high DHT concentrations could affect testosterone's binding to SHBG.

Transcortin and orosomucoid display very low affinity for SHBG; their role in regulating free testosterone was not assessed in this investigation and needs further clarity; it is remarkable that FT concentrations derived using the new dynamic model were not significantly different from those determined by equilibrium dialysis in randomized trials even though inter-individual differences in transcortin, and orosomucoid were not considered, consistent with the view that these proteins play a minor role in regulating free testosterone in healthy men and women.

The current algorithm and the experimental data were generated using wild type SHBG which is present in nearly 98% of Caucasians. Genome wide association studies have revealed several SHBG polymorphisms, two of which affect testosterone's binding to SHBG (Ohlsson et al 2011)(Ohlsson et al 2011). Therefore, in future, the algorithm may include a term for SHBG genotype. Additional research is needed to extend the model to incorporate SHBG polymorphisms that affect testosterone's binding to SHBG.

In summary, experimental data generated using several independent methods provide evidence of an complex allostery mechanism of testosterone binding to SHBG dimer. FT concentrations derived using the new dynamic model incorporating complex allostery do not differ significantly from those measured using equilibrium dialysis. The application of the new dynamic model to clinical trials data have revealed new insights into the percent of circulating testosterone that is free, the relation between percent FT and total testosterone and SHBG. The use of additional experimental models, including dimerization-deficient SHBG mutants, would allow further characterization of testosterone's interaction with SHBG to validate the complex allostery as suggested by this study. The validation of the new dynamic model incorporating complex allostery should also be further explored in clinical populations as its availability on desktops and mobile devices can provide a convenient and accurate approach for determining FT at the point-of-care, and facilitating the diagnosis and treatment of men and women with androgen disorders.

### REFERENCES

Adachi, K., Yasuda, K., Fuwa, Y., Goshima, E., Yamakita, N., Miura, K., 1991. Measurement of plasma free steroids by direct radioimmunoassay of ultrafiltrate in association with the monitoring of free components with [14C]glucose. Clin. Chim. Acta 200, 13-22.
Avvakumov, G. V., Cherkasov, A., Muller, Y. A., Hammond, G. L., 2010. Structural analyses of sex hormone-binding globulin reveal novel ligands and function. Mol. Cell. Endocrinol. 316, 13-23.
Avvakumov, G. V., Grishkovskaya, I., Muller, Y. A., Hammond, G. L., 2002. Crystal structure of human sex hormone-binding globulin in complex with 2-methoxyestradiol reveals the molecular basis for high affinity interactions with C-2 derivatives of estradiol. J. Biol. Chem. 277, 45219-45225.
Avvakumov, G. V., Grishkovskaya, I., Muller, Y. A., Hammond, G. L., 2001. Resolution of the human sex hormone-binding globulin dimer interface and evidence for two steroid-binding sites per homodimer. J. Biol. Chem. 276, 34453-34457.
Avvakumov, G. V., Muller, Y. A., Hammond, G. L., 2000. Steroid-binding specificity of human sex hormone-binding globulin is influenced by occupancy of a zinc-binding site. J. Biol. Chem. 275, 25920-25925.
Bhasin, S., Cunningham, G. R., Hayes, F. J., Matsumoto, A. M., Snyder, P. J., Swerdloff, R. S., Montori, V. M., Task Force, E. S., 2010. Testosterone therapy in men with androgen deficiency syndromes: an Endocrine Society clinical practice guideline. J. Clin. Endocrinol. Metab. 95, 2536-2559.
Bhasin, S., Pencina, M., Jasuja, G. K., Travison, T. G., Coviello, A., Orwoll, E., Wang, P. Y., Nielson, C., Wu, F., Tajar, A., Labrie. F., Vesper. H., Zhang, A., Ulloor. J., Singh, R., D'Agostino, R., Vasan, R. S., 2011. Reference ranges for testosterone in men generated using liquid chromatography tandem mass spectrometry in a community-based sample of healthy nonobese young men in the framingham heart study and applied to three geographically distinct cohorts. J. Clin. Endocrinol. Metab. 96, 2430-2439.
Bhasin, S., Travison, T. G., Storer, T. W., Lakshman, K., Kaushik, M., Mazer, N. A., Ngyuen, A. H., Davda, M. N., Jara, H., Aakil, A., Anderson, S., Knapp, P. E., Hanka, S., Mohammed, N., Daou, P., Miciek, R., Ulloor, J., Zhang, A., Brooks, B., Orwoll, K., Hede-Brierley, L., Eder, R., Elmi, A., Bhasin, G., Collins, L., Singh, R., Basaria, S., 2012. Effect of testosterone supplementation with and without a dual Salpha-reductase inhibitor on fat-free mass in men with suppressed testosterone production: a randomized controlled trial. JAMA 307, 931-939.
Dunn. J. F., Nisula, B. C., Rodbard. D., 1981. Transport of steroid hormones: binding of 21 endogenous steroids to both testosterone-binding globulin and corticosteroid-binding globulin in human plasma. J. Clin. Endocrinol. Metab. 53, 58-68.
Elveback, L., 1973. The population of healthy persons as a source of reference information. Hum. Pathol. 4, 9-16.
Freiburger, L. A., Baettig, O. M., Sprules, T., Berghuis, A. M., Auclair, K., Mittermaier, A. K., 2011. Competing allosteric mechanisms modulate substrate binding in a dimeric enzyme. Nat. Struct. Mol. Biol. 18, 288-294.
Grishkovskaya, I., Avvakumov, G. V., Hammond, G. L., Catalano, M. G., Muller, Y. A., 2002. Steroid ligands bind human sex hormone-binding globulin in specific orientations and produce distinct changes in protein conformation. J. Biol. Chem. 277, 32086-32093.
Grishkovskaya, I., Avvakumov, G. V., Sklenar, G., Dales, D., Hammond, G. L., Muller, Y. A., 2000. Crystal structure of human sex hormone-binding globulin: steroid transport by a laminin G-like domain. EMBO J. 19, 504-512.
Grishkovskaya, 1., Sklenar, G., Avvakumov, G. V., Dales. D., Behlke, J., Hammond, G. L., Muller, Y. A., 1999. Crystallization of the N-terminal domain of human sex hormone-binding globulin, the major sex steroid carrier in blood. Acta Crystallogr. D Biol. Crystallogr. 55, 2053-2055.
Hammond, G. L., Bocchinfuso, W. P., 1996. Sex hormone-binding globulin: gene organization and structure/function analyses. Horm. Res. 45, 197-201.
Hauptmann, H., Metzger, J., Schnitzbauer, A., Cuilleron, C. Y., Mappus, E., Luppa, P. B., 2003. Syntheses and ligand-binding studies of I alpha- and 17 alpha-aminoalkyl dihydrotestosterone derivatives to human sex hormone-binding globulin. Steroids 68, 629-639.
Hilser, V. J., Thompson, E. B., 2007. Intrinsic disorder as a mechanism to optimize allosteric coupling in proteins. Proc. Natl. Acad. Sci. U.S.A. 104, 8311-8315.
Huang, G., Basaria, S., Travison, T., Ho, M., Davda, M., Mazer, N., Knapp, P., Collins, L., Ursion, M., Zhang, A., Ulloor, J., Storer, T., Bhasin, S., 2012. Testosterone dose response in surgically menopausal women. The Endocrine Society Meeting June.
Koshland, D. E., Jr, Nemethy, G., Filmer, D., 1966. Comparison of experimental binding data and theoretical models in proteins containing subunits. Biochemistry 5, 365-385.
Krasnoff, J. B., Basaria. S., Pencina, M. J., Jasuja, G. K., Vasan, R. S., Ulloor. J., Zhang, A., Coviello, A., Kelly-Hayes. M., D'Agostino, R. B., Wolf, P. A., Bhasin, S., Murabito, J. M., 2010. Free testosterone levels are associated with mobility limitation and physical performance in community-dwelling men: the Framingham Offspring Study. J. Clin. Endocrinol. Metab. 95, 2790-2799.
Lee, D. M., O'Neill, T. W., Pye, S. R., Silman, A. J., Finn, J. D., Pendleton, N., Tajar, A., Bartfai, G., Casanueva, F., Forti, G., Giwercman, A., Huhtaniemi, I. T., Kula, K., Punab, M., Boonen, S., Vanderschueren, D., Wu, F. C., EMAS study group, 2009. The European Male Ageing Study (EMAS): design, methods and recruitment. Int. J. Androl. 32, 11-24.
Ly, L. P., Handelsman, D. J., 2005. Empirical estimation of free testosterone from testosterone and sex hormone-binding globulin immunoassays. Eur. J. Endocrinol. 152, 471-478.
Ly, L. P., Sartorius, G., Hull, L., Leung, A., Swerdlolf, R. S., Wang, C., Handelsman, D. J., 2010. Accuracy of calculated frcce testosterone formulae in men. Clin. Endocrinol. (Oxf) 73, 382-388.
Mazer, N. A., 2009. A novel spreadsheet method for calculating the free serum concentrations of testosterone, dihydrotestosterone, estradiol, estrone and cortisol: with illustrative examples from male and female populations. Steroids 74, 512-519.
Mendel, C. M., 1989. The free hormone hypothesis: a physiologically bused mathematical model. Endocr. Rev. 10, 232-274.
Metzger, J., Schnitzbauer, A., Meyer, M., Soder, M., Cuilleron, C. Y., Hauptmann. H., Huber, E., Luppa, P. B., 2003. Binding analysis of 1alpha- and 17alpha-dihydrotestosterone derivatives to homodimeric sex hormone-binding globulin. Biochemistry 42, 13735-13745.
MONOD, J., WYMAN, J., CHANGEUX, J. P., 1965. On the Nature of Allosteric Transitions: a Plausible Model. J. Mol. Biol. 12, 88-118.
Morales, A., Collier, C. P., Clark, A. F., 2012. A critical appraisal of accuracy and cost of laboratory methodologies for the diagnosis of hypogonadism: the role of free testosterone assays. Can. J. Urol. 19, 6314-6318.
Morley, J. E., Patrick, P., Perry, H. M., 3rd, 2002. Evaluation of assays available to measure free testosterone. Metabolism 51, 554-559.
Nanjee, M. N., Wheeler, M. J., 1985. Plasma free testosterone—is an index sufficient? Ann. Clin. Biochem. 22 (Pt 4), 387-390.
Ohlsson, C., Wallaschofski, H., Lunetta, K. L., Stolk. L., Perry, J. R., Koster, A., Petersen, A. K., Eriksson, J., Lehtimaki, T., Huhtaniemi, L T., Hammond, G. L., Maggio. M., Coviello, A. D., EMAS Study Group, Ferrucci, L., Hcicr, M., Hofman, A., Holliday, K. L., Jansson, J. O., Kahonen, M., Karasik. D., Karlsson, M. K., Kiel, D. P., Liu, Y., Ljunggren, O., Lorentzon, M., Lyytikainen, L. P., Meitinger, T., Mellstrotn, D., Melzer, D., Miljkovic, I., Nauck, M., Nilsson, M., Penninx, B., Pye, S. R., Vasan, R. S., Reineke, M., Rivadeneira, F., Tajar, A., Teumer, A., Uitterlinden, A. G., Ulloor, J., Viikari, J., Volker, U., Volzke, H., Wichmann, H. E., Wu. T. S., Zhuang, W. V., Ziv, E., Wu, F. C., Raitakari, O., Eriksson, A., Bidlingmaier, M., Harris, T. B., Murray, A., de Jong, F. H., Murabito, J. M., Bhasin. S., Vandenput, L., Haring, R., 2011. Genetic determinants of serum testosterone concentrations in men. PLoS Genet. 7, e1002313.
Petra, P. H., Namkung, P. C., Senear, D. F., McCrae, D. A., Rousslang, K. W., Teller, D. C., Ross, J. B., 1986. Molecular characterization of the sex steroid binding protein (SBP) of plasma. Re-examination of rabbit SBP and comparison with the human, macaque and baboon proteins. J. Steroid Biochem. 25, 191-200.
Rosner, W., 1997. Errors in the measurement of plasma free testosterone. J. Clin. Endocrinol. Metab. 82, 2014-2015.
Rosner, W., 1991. Plasma steroid-binding proteins. Endocrinol. Metab. Clin. North Am. 20, 697-720.
Rosner, W., Auchus, R. J., Azziz, R., Sluss, P. M., Raff, H., 2007. Position statement: Utility, limitations, and pitfalls in measuring testosterone: an Endocrine Society position statement. J. Clin. Endocrinol. Metab. 92, 405-413.
Sartorius, G., Ly, L. P., Sikaris, K., McLachlan, R., Handelsman, D. J., 2009. Predictive accuracy and sources of variability in calculated free testosterone estimates. Ann. Clin. Biochem. 46, 137-143.
Sinha-Hikim. I., Arver, S., Beall, G., Shen, R., Guerrero, M., Sattler, F., Shikuma. C., Nelson, J. C., Landgren, B. M., Mazer, N. A., Bhasin, S., 1998. The use of a sensitive equilibrium dialysis method for the measurement of free testosterone levels in healthy, cycling women and in human immunodeficiency virus-infected women. J. Clin. Endocrinol. Metab. 83, 1312-1318.
Sodergard, R., Backstrom, T., Shanbhag, V., Carstensen, H., 1982. Calculation of free and bound fractions of testosterone and estradiol-17 beta to human plasma proteins at body temperature. J. Steroid Biochem. 16, 801-810.
Solberg. H. E., 1987. International Federation of Clinical Chemistry (IFCC), Scientific Committee, Clinical Section, Expert Panel on Theory of Reference Values, and International Committee for Standardization in Haematology (ICSH), Standing Committee on Reference Values. Approved Recommendation (1986) on the theory of reference values. Part 1. The concept of reference values. J. Clin. Chem. Clin. Biochem. 25, 337-342.
Spitzer, M., Basaria, S., Travison, T. G., Davda, M. N., Paley, A., Cohen, B., Mazer, N. A., Knapp, P. E., Hanka, S., Lakshman, K. M., Ulloor. J., Zhang, A., Orwoll, K., Eder, R., Collins, L., Mohammed, N., Rosen, R. C., DeRogatis, L., Bhasin, S., 2012. Effect of testosterone replacement on response to sildenafil citrate in men with erectile dysfunction: a parallel, randomized trial. Ann. Intern. Med. 157, 681-691.
Splansky, G. L., Corey, D., Yang, Q., Atwood, L. D., Cupples, L. A., Benjamin. E. J., D'Agostino R B, S., Fox, C. S., Larson, M. G., Murabito, J. M., O'Donnell, C. J., Vasan, R. S., Wolf, P. A., Levy, D., 2007. The Third Generation Cohort of the National Heart, Lung, and Blood Institute's Framingham Heart Study: design, recruitment, and initial examination. Am. J. Epidemiol. 165, 1328-1335.
Tajar, A., Forti, G., O'Neill, T. W., Lee, D. M., Silman, A. J., Finn, J. D., Bartfai, G., Boonen, S., Casanueva. F. F., Giwercman. A., Han, T. S., Kula, K., Labrie, F., Lean. M. E., Pendleton, N., Punab, M., Vanderschueren, D., Huhtaniemi, I. T., Wu, F. C., EMAS Group, 2010.
Characteristics of secondary, primary, and compensated hypogonadism in aging men: evidence from the European Male Ageing Study. J. Clin. Endocrinol. Metab. 95, 1810-1818.
Van Uytfanghe, K., Stockl, D., Kaufman, J. M., Fiers, T., Ross, H. A., De Leenheer, A. P., Thienpont, L. M., 2004. Evaluation of a candidate reference measurement procedure for serum free testosterone based on ultrafiltration and isotope dilution-gas chromatography-mass spectrometry. Clin. Chem. 50, 2101-2110.
Vermeulen, A., Stoica, T., Verdonck, L., 1971. The apparent free testosterone concentration, an index of androgenicity. J. Clin. Endocrinol. Metab. 33, 759-767.
Vermeulen, A., Verdonck, L., Kaufman, J. M., 1999. A critical evaluation of simple methods for the estimation of free testosterone in scrum. J. Clin. Endocrinol. Metab. 84, 3666-3672.
Winters, S J., Kelley, D. E., Goodpaster, B., 1998. The analog free testosterone assay: are the results in men clinically useful? Clin. Chem. 44, 2178-2182.
Wood, S. N., 2006. Generalized Additive Models: An Introduction with R, pp. 391, Chapman & Hall/CRC, Boca Raton, Fla.
Wu, F. C., Tajar, A., Beynon, J. M., Pye, S. R., Silman, A. J., Finn, J. D., O'Neill, T. W., Bartfai, G., Casanueva, F. F., Forti, G., Giwercman, A., Han. T. S., Kula, K., Lean, M. E., Pendleton, N., Punab, M., Boonen, S., Vanderschueren, D., Labrie, F., Huhtaniemi, I. T., EMAS Group, 2010. Identification of late-onset hypogonadism in middle-aged and elderly men. N. Engl. J. Med. 363, 123-135.
Zakharov, M. N., Bhasin, S., Szafran, A. T., Mancini, M. A., Jasuja, R., 2012. Numerical framework to model temporally resolved multi-stage dynamic systems. Comput. Methods Programs Biomed. 108, 750-759.

### Example 3

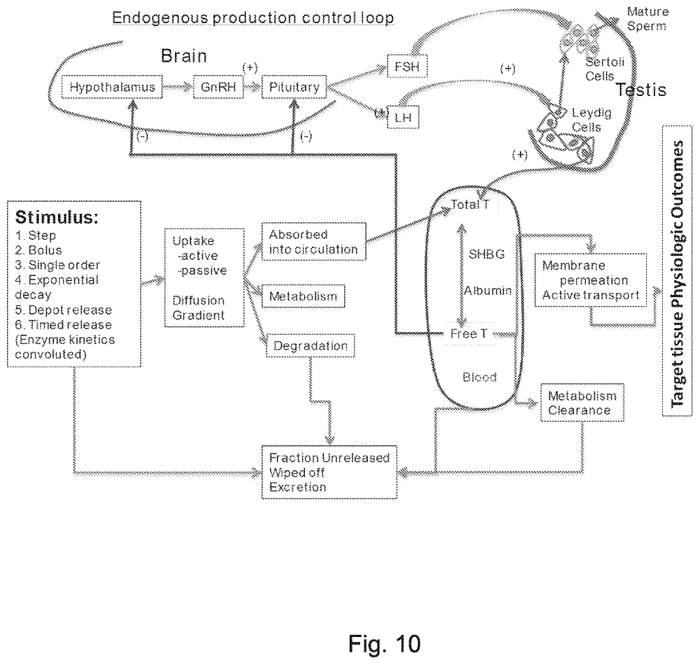

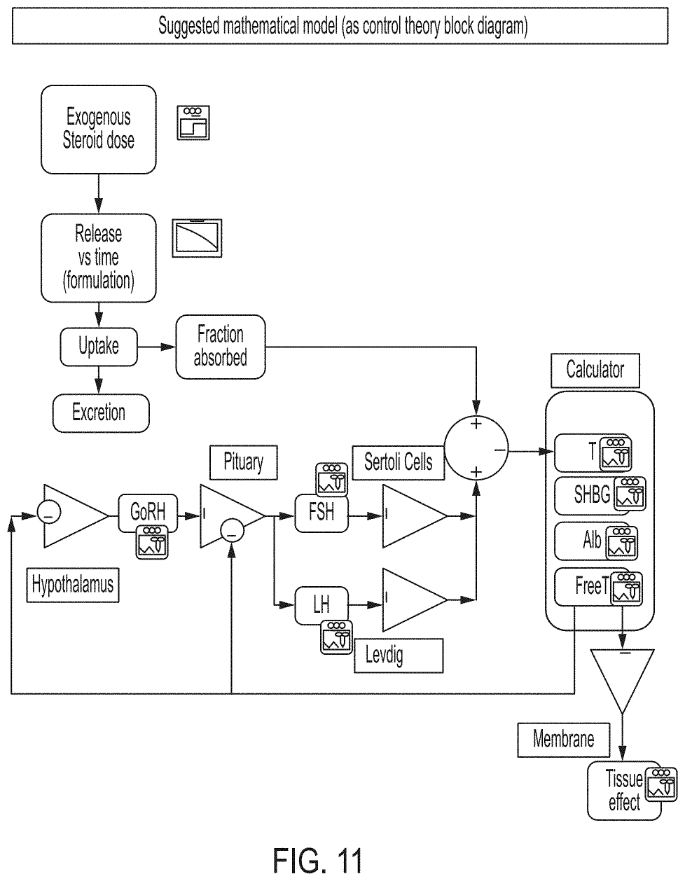

In one aspect, described herein is a personalized exogenous steroid delivery dosimeter. Time dependent drug delivery/clearance models can be incorporated with the calculator of free T described herein (FIG. 10). T administration, redistribution, and clearance rates can be accounted for. In some embodiments, an experiment, a model, and set of parameters are present and the model described herein can be used to obtain results. In some embodiments, a set of datasets and experiments are present, and the model described herein can be used to determine initial model parameters.

The methods and systems described herein can include total T level. SHBG level, albumin level, SHB polymorphisms, clearance rates, circadian rhythms, metabolism levels. LH level, FSH level, T degradation, T diffusion/permeability coefficient, and release speed.

### Example 4

The Endocrine Society acknowledges that the methods available for free-T testing are fraught with error and recommends the usage of calculated free-T levels in clinical practice pertaining to mens' health. Research publications and the analyses described herein have confirmed that current free-T calculation methodologies are plagued with bias and variance. A popular algorithm likely overestimates the free-T level and induces uncertainty that reduces the likelihood of treatment initiation.

Hypogonadism in men is a clinical syndrome that resus from failure of the testes to produce physiological levels of testosterone (androgen deficiency) and a normal number of spermatozoa due to disruption of one or more levels of the hypothalamic-pituitary-testicular (HPT) axis. Testosterone therapy is indicated for the treatment of men with classical androgen deficiency syndromes and aims to induce and maintain secondary sex characteristics and improve sexual function, sense of well-being, and bone mineral density.

A number of testosterone formulations have been approved for the treatment of hypogonadism in men, including injectable testosterone esters, testosterone transdermal patch, transdermal testosterone gels, buccal adhesive testosterone tablets, and testosterone pellets. While transdermals represented 80% of an almost S2.2 Billion market in 2012, little has been done to alleviate the marked inter-individual variability in serum testosterone levels in hypognadal men on testosterone replacement therapy (TRT). This variability has been particularly striking with the transdermal gel formulations and data suggests that more than 50% of men on the recommended initial 5 g/1% testosterone gel do not achieve testosterone levels in the target range. Additionally, approximately 10 to 15% of treated men will have testosterone levels above the upper limit of the normal range, rendering them at risk for adverse events. Interestingly, it is widely proven that Testosterone prescription use indicates that a large fraction (nearly 50%) of men that are started on testosterone therapy discontinue testosterone treatment within 1.0 to 3.5 months and never re-fill their prescription.

A survey of hypogonadal men treated in the Northeastern VA system also reveals a median DOT of 28 days. Thus, a majority of hypogonadal men started on testosterone therapy discontinue testosterone therapy within 3.5 months. It is contemplated herein that a major cause of treatment discontinuation is the failure to achieve the desired therapeutic effect because of suboptimal on-treatment testosterone levels. Monitoring of on-treatment free testosterone levels and appropriate adjustment of testosterone dose to achieve therapeutic levels are necessary for achieving optimal treatment effects and retaining patients on therapy.

The methods, assays, and systems described herein permit the calculation of free testosterone concentrations based on serum total testosterone and SHBG concentrations, and provide specific guidance on dose adjustment needed to achieve the target free testosterone concentration. They further provide a mechanism for enhancing physician-patient engagement, aid the practicing clinician in optimizing the TRT dose to achieve desired on-treatment free testosterone levels that improves treatment outcomes, and retain patients on therapy for a significantly longer period of time than the current median duration.

### Example 5

Binding proteins (e.g. SHBG, albumin, orosomucoid and transcortin) and testosterone (T) dynamically interact in multiple steps to regulate testosterone availability.

Circulating testosterone is bound mostly to plasma proteins, sex-hormone binding globulin (SHBG) and albumin. There are several states in which the testosterone and binding proteins are distributed. They continually re-partition into a series of states that are in conformational equilibria. The populations of the intermediate states redistribute as the concentrations of testosterone and binding proteins change. In one of its embodiments (as an example), SHBG exists in two distinct states in the solution governed by a unimolecular equilibrium constant. Both states are capable of binding T and upon binding T, they proceed to the corresponding states with one monomer bound to T and the other monomer is unoccupied within the dimer. The binding of first T induces allosteric changes in monomer that is still unoccupied and therefore results in a distinct change in affinity for the second molecule of T for the SHBG dimer. Consistent with the crystal structure of liganded SHBG, Binding of the second molecule to either of the intermediates results in the identical state of fully occupied SHBG dimer.

In one embodiments, free testosterone is calculated as described below.

The relative population of the intermediates is closely coupled through the multiple equilibria and dynamically readjust. Accordingly, testosterone's binding to SHBG cannot be described as a simple linear equation of ligand binding equilibrium. Described herein is a multi-species allostery model, e.g., in LabVIEW framework, that fits the experimental data and permits the development of a set of parameters that accurately described the multiple interactions listed below:

$$S T + {S}_{2} T + {S}_{1} T + {S}_{1} + S = {S}_{0}$$

$$T + S T + 2 \cdot {S}_{2} T + {S}_{1} T + {T}_{f} + albT = {T}_{0}$$

$$T = {T}_{f} \\ - {k}_{1} + S \cdot T + {k}_{1 -} S T + {k}_{11 -} {S}_{1} - {k}_{11} + S = 0 \\ - {k}_{3 +} {S}_{1} \cdot T + {k}_{3 -} {S}_{1} T - {k}_{11 -} {S}_{1} + {k}_{11 +} S = 0 \\ - {k}_{2 +} S T \cdot T + {k}_{1 +} S \cdot T - {k}_{1 -} S T + {k}_{2 -} {S}_{2} T = 0$$

$${k}_{2 +} S T \cdot T + {k}_{4 +} {S}_{1} T \cdot T - ( {k}_{4 -} + {k}_{2 -} ) \cdot {S}_{2} T = 0$$

$$albT + alb = {alb}_{0}$$

Using a set of equilibrium constants derived from the fits to the biophysical data provided in FIGS. 5A, 5B, and 5C, an exemplary solution of the series of equations is presented below to calculate free T. The solution below doesn't imply presence of a unique equation rather that distinct equations can be developed to achieve accurate calculation of free T with a model of interaction that involves (and is not limited to) inter-subunit allostery in SHBG, regulation of free T levels by albumin and conformational states of binding proteins (albumin and SHBG).

Tf*(4311602270490*T_0-792951348000*S_0+95385510*Alb0-5341588560000)+Tf\hat{ }2*(916102610798*T_0-210702480309*S_0+153989202*Alb0-8623204540980)+Tf\hat{ }3*(18366076550*T_0-22591273900*S_0+32723450*Alb0-1832205221596)+Tf\hat{ }4*(1539342900*T_0-3079192900*S_0+657100*Alb0-36732153100)+2670794280000*T_0+Tf\hat{ }5*(55000*T_0+110000*S_0-55000*Alb0-3078685800)+110000*Tf\hat{ }6=0

In a number of disease states and conditions, the concentrations of binding proteins are altered and the determination of free testosterone is necessary for making a diagnosis of hypogonadism and evaluation of gonadal status. The common conditions in which SHBG concentrations are decreased include but are not limited to obesity, diabetes mellitus, and hypothyroidism. Correspondingly there are patient symptoms that may be clinical or subclinical presentation of Androgen related disorders that may alter the Testosterone and SHBG concentrations such as hirsutism, muscular dystrophy, Androgen insensitivity, acne, polycystic ovarian syndrome. Acromegaly, Anorexia, Androgen expressing tumors etc. It is specifically contemplated herein that the methods, systems, and assays described herein can be used either for patients diagnosed with an androgen disorder or patients who are suspected of having an androgen disorder. e.g. those subjects exhibiting one or more symptoms or risk factors for an androgen disorder. It is specifically contemplated herein that the methods, systems, and assays described herein can be used either for patients diagnosed with a disease or condition that arises from or is characterized by an abnormal level of testosterone or SHBG or patients who are suspected of having a disease or condition that arises from or is characterized by an abnormal level of testosterone or SHBG. e.g. those subjects exhibiting one or more symptoms or risk factors for a disease or condition that arises from or is characterized by an abnormal level of testosterone or SHBG.

Similarly, SHBG concentrations are increased in hyperthyroidism, chronic infections such as HIV, and hepatitis B and C. and old age. Albumin concentrations are decreased in cancer cachexia, malnutrition, liver disease, nephrotic syndrome, and in chronic infections. It is specifically contemplated herein that the methods, systems, and assays described herein can be used either for patients diagnosed with a disease or condition that arises from or is characterized by an abnormal level of albumin or SHBG or patients who are suspected of having a disease or condition that arises from or is characterized by an abnormal level of albumin or SHBG, e.g. those subjects exhibiting one or more symptoms or risk factors for a disease or condition that arises from or is characterized by an abnormal level of albumin or SHBG.

In all these conditions, total testosterone concentrations are affected by the changes in the binding protein concentrations and do not accurately reflect androgen status. Therefore, in these conditions, the determination of free testosterone concentrations is essential for accurately assessing androgen status and making an accurate diagnosis of hypogonadism or androgen excess. Therefore, in evaluating patients for hypogonadism or androgen deficiency, pubertal disorders, hirsutism, androgenic alopecia, infertility, or gynecomastia, who have one or more of the conditions listed above that alter binding protein concentrations, an accurate determination of free testosterone concentrations is necessary.

The diagnosis of hypogonadism is based on ascertainment of low total testosterone levels, which can be misleading in conditions listed above in which binding protein concentrations are affected. Therefore, in these patients with alterations in binding protein concentrations, the diagnosis should be based on free testosterone Levels. The Endocrine Society has published cut-off levels that define low free testosterone levels (Bhasin et al, Testosterone Therapy of Men with Androgen Deficiency Syndromes: An Endocrine Society Guideline. JCEM 2010). These cut-off levels for free testosterone were based on methods which are demonstrated herein to be inaccurate Using the methods, assays, and/or systems described herein, new cut-offs for defining low free testosterone in men in different decades of age are provided herein. These reference values will facilitate accurate diagnosis of hypogonadism in men. Based on the distribution of free testosterone in men, the lower limit of the normal range is determined to be 114.6 pg/mL.

Using the methods, assays, and systems described herein, the optimal range of free testosterone concentrations that should be targeted in hypogonadal men receiving testosterone replacement therapy have also been determined. The treatment of hypogonadism with testosterone is currently suboptimal. The analyses of clinical trials data described herein demonstrate that a large fraction of hypogonadal men treated with testosterone therapy have testosterone levels in the subtherapeutic range.

The current Endocrine Society guidelines suggest the use of total testosterone levels to guide therapy, which as discussed above, do not provide an accurate assessment of the androgen status. The free testosterone concentrations, determined the new method described herein, can provide accurate assessment of the adequacy of testosterone therapy in hypogonadal men. Based on the new data on the distribution of free testosterone levels in healthy men, the target range of free testosterone has been determined to be 164 to 314 pg/ml (mean+/−1SD). If the on-treatment free testosterone concentrations determined using the methods described herein are outside this range, the dose of testosterone should be adjusted using the methods described herein to achieve testosterone levels in the target therapeutic range to maximize benefits and reduce the risks. Furthermore, the initial dose of testosterone therapy can be determined using the methods, assays, and/or systems described herein, e.g. the dosimeter methods described herein.

In some embodiments, the methods, assays, and/or systems described herein permit the determination or measurement of free testosterone in a subject and the determination of a dose of testosterone. In some embodiments, the subject can be classified, e.g., as having low or normal testosterone levels, as needing or not needing testosterone therapy, or as at risk or not at risk of having or developing an androgen disorder. In some embodiments, additional clinical symptoms can be used in classifying a subject. In some embodiments, multiple solutions can be found for the set of equations described herein, dependent upon accuracy vs. computational intensiveness. In some embodiments, the model described herein can be subjected to spline-based linearization based on marker ranges.

In some embodiments, the methods, assays, and/or systems described herein can comprise the calculation of a suitable dosage and/or determining the effect an existing dosage has on the subject's free testosterone levels. In some embodiments, the methods, assays, and/or systems described herein can comprise adjusting a subject's dose, e.g., to a dose that will cause their free testosterone levels to be normal. In some embodiments, the adjustment can be step-wise adjustment. In some embodiments, the adjustment can comprise multiple steps or changes in order to reach a final or target dosage. In some embodiments, the methods, assays, and/or systems described herein can comprise conversion of a calculated dose to available formulations. e.g., determining the dosage present in a given formulation that is necessary in order to deliver a particular dosage or maintain a particular dosage in a subject. In some embodiments, the methods, assays, and/or systems described herein can comprise dosing changes that are counterintuitive due to control loop characterization and for other known or unknown physiological, behavioral, psychological or environmental reasons. In some embodiments, the methods, assays, and/or systems described herein can comprise additional steps to characterize, e.g., individual clearance rates, individual uptake rates (e.g., skin permeability), or other factors. e.g. changes in metabolism or biochemistry caused by other ailments, conditions, or environmental changes that influence the testosterone balance and/or levels. In some embodiments, the subject can be a hard to treat subject, e.g., one that does not respond normally to testosterone therapy.

### Example 6: Estradiol Binding Induces Bidirectional Allosteric Coupling and Repartitioning of Sex Hormone Binding Globulin Monomers Among Various Conformational States

Sex hormone-binding globulin (SHBG) regulates the transport, bioavailability, and metabolism of estradiol. Estradiol's binding to SHBG is non-linear and “apparent” Kd changes with varying estradiol and SHBG concentrations. Estradiol's binding to the first SHBG monomer influences residues in ligand binding pockets (LBP) of both monomers, and differentially alters the conformational and energy states of each of the two monomers. Furthermore, estradiol's binding to second monomer impacts energy landscape of the first, estradiol-bound monomer.

Estradiol's binding to SHBG dimer was shown to involve bidirectional inter-monomeric allostery that changes the energy landscape of both monomers and their distribution among various conformational states; monomers are not equivalent energetically or conformationally even in fully-bound state. Inter-monomeric allostery offers a mechanism to extend SHBG's binding-range and regulate hormone-bioavailability as estradiol concentrations vary widely during life.

### Introduction

Estradiol (E2), the dominant estrogen in men and women, is found in human circulation bound primarily to sex hormone-binding globulin (SHBG) and human serum albumin (HSA) [1A-8A]. These circulating binding proteins regulate the transport, bioavailability, and metabolism of estradiol [9A-16A], and the biological activity of the circulating hormone is related to the fraction that crosses into the tissue.

As described herein, multiple biophysical methods, modern computational tools, and molecular modeling were used to examine nonlinear data derived from the binding isotherms and depletion curves to gain a better understanding of the dynamics of estradiol's binding to SHBG. Liquid chromatography tandem mass spectrometry (LC-MS/MS) was used to directly measure estradiol concentrations on both sides of the dialysis chamber and employed standardized dialysis conditions at 37° C. The findings of the equilibrium dialysis experiments were corroborated with steady-state and singlet-excited state fluorescence spectroscopy. A wide range of estradiol concentrations extending from subphysiologic to supraphysiologic range were used. Molecular modeling studies of estradiol's binding to SHBG were performed over five micro-seconds to probe the binding behavior at the amino acid level. Estradiol's binding to SHBG was found to be a nonlinear, dynamic process, involving allosteric coupling between the SHBG monomers that changes the energy landscape of both monomers and their distribution between various energy states such that the two monomers are not equivalent even in the fully bound state.

### Materials and Methods

Equilibrium dialysis measurements. Purified SHBG was obtained from The Binding Site (product code BH089.X, The Binding Site, San Diego, Calif.). SHBG concentrations in the stock solutions were measured using a Nanodrop ND-1000 Spectrophotometer (ThermoFisher Scientific, Waltham, Mass.). Absorption was read at 278 nm, the path length was 1 cm, and an extinction coefficient of 56740 M−1 cm−1 was used [29A].

The dialysis buffer composition simulated the ionic strength conditions in the blood: 90 mM NaCl, 3 mM KCl, 1.3 mM KH2PO4, 1.9 mM CaCl2.2H2O, 1.1 mM MgSO4.7H2O, 5 mM 0.30 g urea and 23 mM HEPES sodium salt, 30 mM HEPES acid, 8 mM sodium azide, and 1 mL of 0.06% DL lactic acid. Equilibrium dialysis was performed in 96-well plates (Harvard Apparatus, Holliston, Mass.) with semi-permeable membranes that allow species >10 kDa to pass through [30A]. 200 μL of dialysis buffer was added to the “buffer side”, and estradiol and SHBG were added to the “sample side” in a total volume of 200 μL. Dialysis plates were incubated for 24 hours at 37° C., after which 150 μL aliquots were removed from each side for estradiol measurement using a validated LC-MS/MS assay that is certified by the Center for Disease Control's Hormone Standardization Program (HoST) [31A]. The lower limit of quantitation of estradiol LC-MS/MS assay was 1 pg·mL−1, the linear range from 1 to 500 pg/mL, and the intra- and inter-assay coefficients of variation were less than 10%.

Fluorescence spectroscopy experiments to examine estradiol-induced perturbations in SHBG. Fluorescence spectroscopy studies were performed using a K2 Multifrequency Phase Fluorometer (ISS, Champaign, Ill.) in 5 mm×10 mm quartz cuvettes (Starna Cells, Atascadero, Calif.). Steady-state measurements were performed using an ISS lamp (model 90513) with the current set to 20 A and a photomultiplier tube (PMT) voltage set to 6.5 V. The excitation wavelength was set to 290 nm with a light-path slit width of 2 mm. To correct for Wood's anomaly, a polarizer oriented at 0° was placed in the emission path. Inner filter effects were negligible for the concentrations used. The emission wavelength range was scanned from 314 to 400 nm with a 2 nm step size.

bis-ANS (Sigma Aldrich, St. Louis, Mo.) was used as an extrinsic probe [30A] in fluorescence lifetime experiments. The solid powder was initially reconstituted in PBS to a concentration of 3.2 mM (the extinction coefficient, ε, is 16,790 cm²·mmol⁻¹⁻ at 385 nm [30]) and stored at room temperature away from light. This parent stock was diluted with PBS to a working stock concentration used for each experiment. For lifetime measurements, the same ISS K2 Multifrequency Phase Fluorometer was used, but the excitation source was a 370 nm light-emitting diode (LED) to excite bis-ANS. Emission of bis-ANS was observed through a 420 nm long-pass filter (Newport Corporation, Irvine, Calif.). In the multifrequency phase and modulation technique, the intensity of the excitation light is modulated from 10 to 160 MHz (with a total of 10 points), the phase shift and relative modulation of the emitted light (with respect to the excitation) are determined, and the lifetimes are found using well-established equations. The phase and modulation were analyzed as a sum of exponentials using a nonlinear least squares procedure implemented using ISS Vinci software and the goodness of fit to the data of a specific model was assessed using the value of the reduced chi-square.

Molecular dynamics studies. Structure of SHBG complexed with estradiol was retrieved from Research Collaboratory for Structural Bioinformatics (RCSB-PDB ID: 1LHU, Uniprot ID: P04278) [33A, 34A]. The crystallographic water was removed and hydrogens were added to the structure. Amino acids were then analyzed for correct protonation states using H++ server [35A]. The processed structure was used to make Apo SHBG dimer, singly-bound SHBG dimer and doubly-bound SHBG dimer with estradiol. The complexes were then prepared for molecular dynamics studies.

Each complex was parametrized employing ff14SB [36A] force field and solvated using TIP3P [37A] water molecules. Minimum distance of 10 A was kept between any solute atom and the box boundary. Electroneutrality of solvated complex was achieved by adding Na+ ions. Periodic boundary conditions were applied along with PME summation [38A] for non-bonded calculations. SHAKE was applied to covalently bound hydrogen atoms with 2 fs time steps. 10 A cut-off was used for non-bonded interaction calculations [39A]. Berenson thermostat [39A] was utilized for creating constant pressure conditions. Each solvated complex was energy minimized in steps with decreasing force on protein residues. Final unrestricted minimization was carried out with 1000 steps of steepest descent followed by 2000 steps of conjugate gradient. Complex was then heated to 300K with complex fixed with restraint force of 25 kcal·mol−1. Å−2. Temperature was maintained by Langevin thermostat [40A]. Equilibration was performed by slowly decreasing the restraint force on the complex from 25 kcal·mol−1. Å−2 till 0.1 kcal·mol−1. Å−2. The resulting structure was then equilibrated for 20 ns at 300 K with no restraining force. Thermodynamic parameters were observed for stability and fluctuations. Production run was carried out in NPT conditions for 5 μs. Complex preparation and Molecular dynamics (MD) simulations were carried out using AMBER v18 [41A] suite of programs for MD. MD was run on CUDA enabled NVidia GPUs employing pmemd [42A, 43A] implementation in AMBER. Analyses reported were performed using self-written scripts/codes and [44A] module of AMBER. Visualization and some analyses on structures utilized VMD [45A] and Pymol [46A].

Markov State Model construction from clustering and network analysis. Markov state models were constructed by clustering SHBG trajectories into sub-states based on the root mean square deviation (RMSD). All trajectories (Apo SHBG, singly bound SHBG and doubly bound SHBG) were stripped of solvent, ions and estradiol. These were concatenated into a single trajectory and used as input for clustering. AMBER implementation of K means clustering was utilized for clustering purposes. The trajectory was partitioned into 2 to 12 clusters and used for analyses of sub-states. The population of each sub-state and rate of its conversion sub-sates to other sub-states was studied using different modules available in AMBER and in-house written scripts/codes. The states and the rate of conversion between states were generated using Python scripts.

### Results

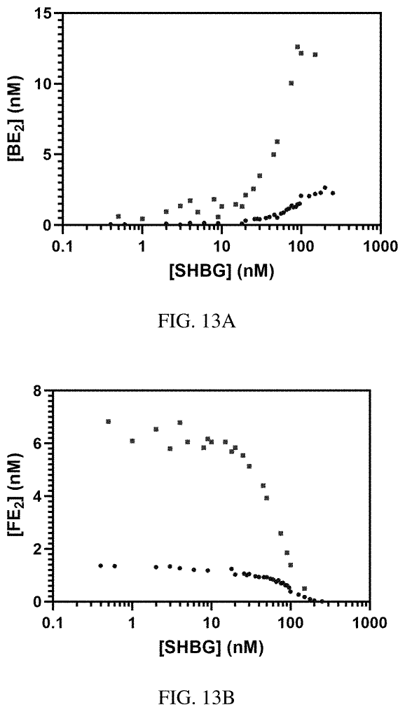

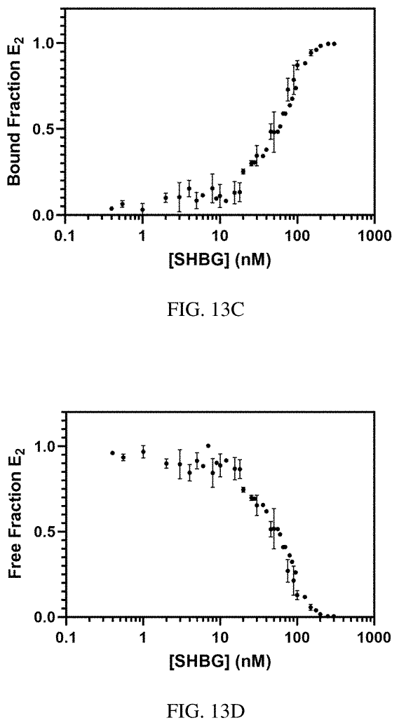

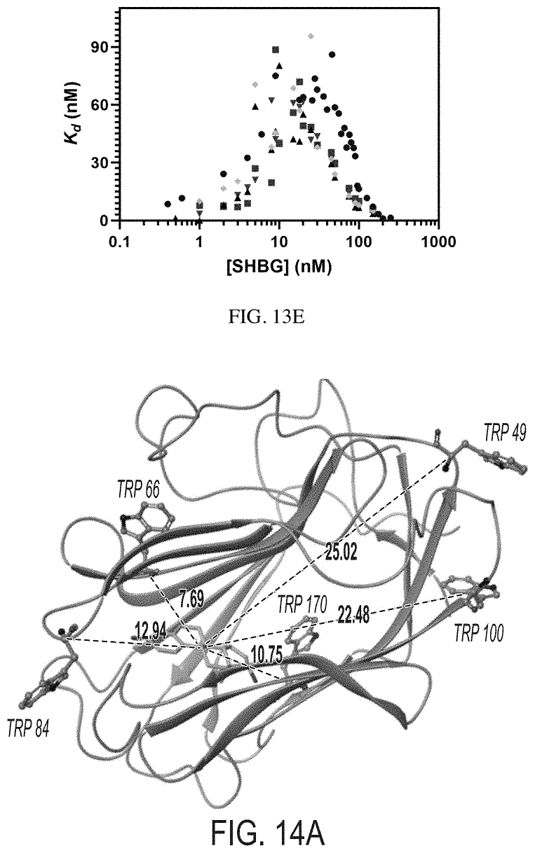

Estradiol binding to SHBG exhibits complex interaction dynamics. Equilibrium dialysis experiments were performed using a wide range of SHBG concentrations at varying estradiol to SHBG ratios and the resulting isotherms for estradiol's binding to SHBG are shown in FIG. 13A. The bound and free estradiol fractions were determined by measuring the estradiol concentrations on each side of the dialysis membrane using LC-MS/MS after overnight incubation (Methods). As SHBG concentration increased, an increasing amount of estradiol partitioned into the sample side. FIG. 13B shows the corresponding depletion curves, which were generated from the decrease in the free estradiol concentrations on the buffer side of the dialysis membrane, as the SHBG concentration was increased. The binding and depletion curves generated by plotting the fraction of bound and free estradiol from two independent experiments are shown in FIGS. 13C and 13D.

The binding isotherm of estradiol's binding to SHBG generated using equilibrium dialysis was not symmetric around the point corresponding to 50% bound estradiol; the complex asymmetry of the binding isotherm is inconsistent with the notion of homogeneous binding of estradiol to SHBG with a single Kd (FIG. 13). From experimentally derived bound and free estradiol concentrations at each SHBG concentration, the “apparent” Kd was computed and plotted against SHBG concentrations (FIG. 13E) at various estradiol concentrations. The apparent Kd varied with the SHBG concentration and the relative ratio of estradiol and SHBG concentrations. The variation in “apparent” Kd at varying concentrations of estradiol and SHBG and at varying estradiol to SHBG ratios indicates that there are multiple equilibria in the estradiol-SHBG interaction with distinct affinities. These data support the view that the two monomers do not have equivalent binding affinity for estradiol. The binding isotherms in FIG. 13A are also inconsistent with two static Kd values, one for each monomer. The binding sites on the two monomers appear to be allosterically coupled to enable dynamic changes in the apparent Kd that is influenced by the relative concentrations of estradiol and SHBG.

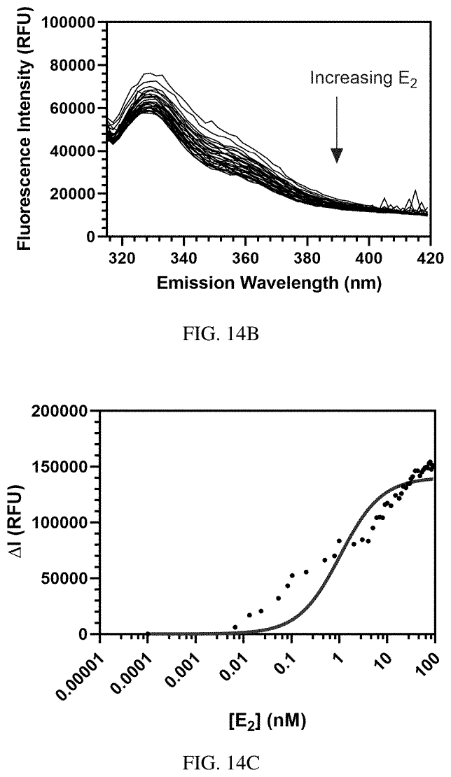

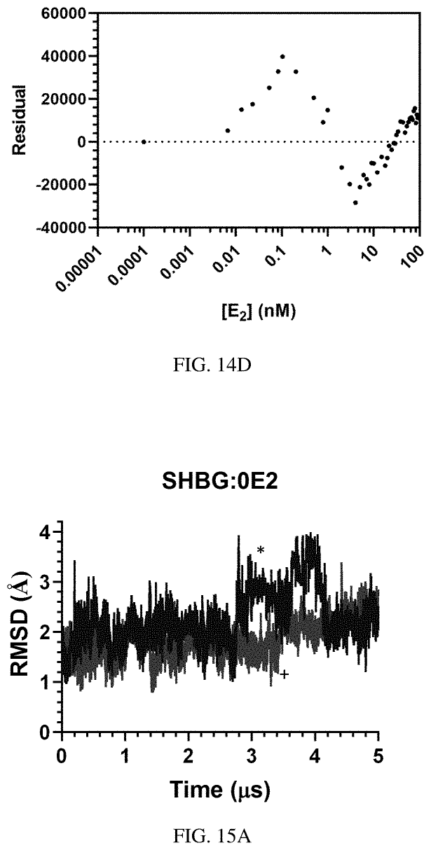

Intrinsic tryptophan emission from SHBG provides evidence that estradiol binding is multiphasic and associated with changes in tryptophan micro-environment. Of the 11 tryptophan residues in the full-length SHBG, the LG domain possesses five tryptophan residues, two of which (W145 and W184) are near the ligand binding pocket (LBP) (FIG. 14A). The perturbations in the emission from the tryptophan residues were used to monitor estradiol's binding to SHBG at graded estradiol concentrations.

FIG. 14B shows the emission spectra of 20 nM SHBG titrated with a wide range of estradiol concentrations that extended from subphysiologic to supraphysiologic range. The system was excited at 290 nm and the emission spectrum was collected from 314 to 420 nm. As increasing concentrations of estradiol were titrated into the SHBG solution, a significant concentration-dependent reduction was observed in intrinsic tryptophan emission. The concentration-dependent increase in quenching of tryptophan fluorescence from SHBG solution suggests that estradiol binding is associated with changes in the tryptophan micro-environment due to conformational rearrangement in SHBG.

In addition to monitoring the perturbations in the fluorescence spectra, integrated emission intensity from tryptophan was independently collected through a long-pass filter (WG305). The emission spectra presented in FIG. 14B were used to generate the binding isotherm that is presented in FIG. 14C. The observed binding isotherm shows that estradiol binding to SHBG is a nonlinear process that does not conform to the isotherm predicted by a linear binding model that assumes a single Kd (the red curve in FIG. 14C). The binding isotherm predicted by the linear binding model with a single Kd (red curve) under-estimates estradiol binding in the lower concentration range and overestimates estradiol binding as the estradiol concentration is increased (FIG. 14D). The binding isotherms generated from intrinsic tryptophan fluorescence quenching independently corroborate the presence of multiple binding equilibria observed previously in the equilibrium dialysis data presented in FIG. 13.

Estradiol-induced molecular rearrangement alters the conformational states of residues in the ligand binding pockets of SHBG monomers, suggesting inter-monomeric allosteric coupling. Molecular modeling studies were performed to evaluate whether estradiol binding to SHBG induces conformational rearrangements in the estradiol binding pocket and in regions distant from the binding pocket. Protein backbone flexibility computations are an important tool to examine the alterations in the equilibrium distribution of protein conformations. The intrinsic conformational flexibility parameters have been recognized as contributors to protein function and are calculated by relaxing the atomic environment starting from the static crystal structure and provide complementary characterization of the conformational perturbations in the estradiol-bound states of SHBG. The estradiol-bound SHBG crystal structure was used to probe the contacts that estradiol makes with the amino acids in the binding pocket and the dimerization interface. Trajectory analysis of the observed alterations in the flexibility of the protein backbone was performed in three populations of SHBG molecules: a) unliganded SHBG dimer (SHBG:0E2); b) single-bound state in which only the ligand binding pocket of the first monomer is occupied (SHBG:1E2) and c) fully-bound SHBG with both binding sites occupied (SHBG:2E2). The structures were allowed to minimize and equilibrate using AMBER suite for Molecular dynamics simulations (ff14SB forcefield, 10 Å cutoff) and trajectories were monitored over 5 micro-seconds. The dynamics of the residue rearrangement calculated for the unliganded SHBG and the estradiol-bound forms of SHBG with one or both the ligand binding pockets (LBP) occupied are reflected in the root-mean-square-deviation (RMSD) plots.

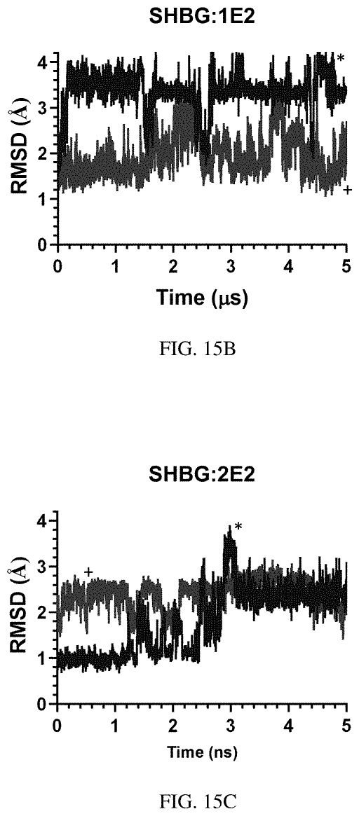

To examine the allosteric coupling between the two SHBG monomers, the LBPs of each monomer (FIGS. 15A, 15B, and 15C) were followed as SHBG progressed from unliganded (SHBG:0E2) to single E2 bound (SHBG:1E2) to doubly bound (SHBG:2E2) states. FIG. 15A shows that even in the unliganded SHBG dimer, the ligand binding pockets in the two monomers populate distinct conformational states. As expected, the conformational flexibility of the ligand binding pocket of monomer 1 was altered upon binding of estradiol to monomer 1 (FIG. 15B, darker trace). Similarly, the conformational states of LBP of monomer 2 are sensitive to the presence of estradiol on the first monomer as evidenced by the altered trajectories of these residues in the singly bound state (FIG. 15B, lighter trace). Of note, the binding of the second estradiol molecule to monomer 2 altered the conformational states in the LBP of monomer 1 which was already occupied by estradiol (FIG. 15C, darker trace).

Time-resolved lifetime fluorescence spectroscopy using bis-ANS demonstrates that estradiol binding significantly alters the global conformational state of the SHBG:E2 complex. The data from the equilibrium dialysis, steady-state fluorescence emission experiments, and molecular modeling studies suggested that estradiol binding sites on the two SHBG monomers are allosterically coupled; the ligand binding to one of the SHBG monomers induced conformational rearrangement that was associated with alterations in the conformation of the second binding site in the SHBG dimer. These findings were further validated by examining the partitioning of a hydrophobic fluorescent probe, bis-ANS (4,4′-dianilino-1,1′-binaphthyl-5,5′-disulfonic acid), into the interior of SHBG. bis-ANS has been used extensively as a sensitive probe to interrogate the steady-state as well as the kinetics of ligand-induced conformational changes in receptor proteins. Accordingly, changes in the solvent accessible surface area and global conformational changes were measured in the SHBG protein as graded concentrations of estradiol were added to a solution containing bis-ANS and SHBG.

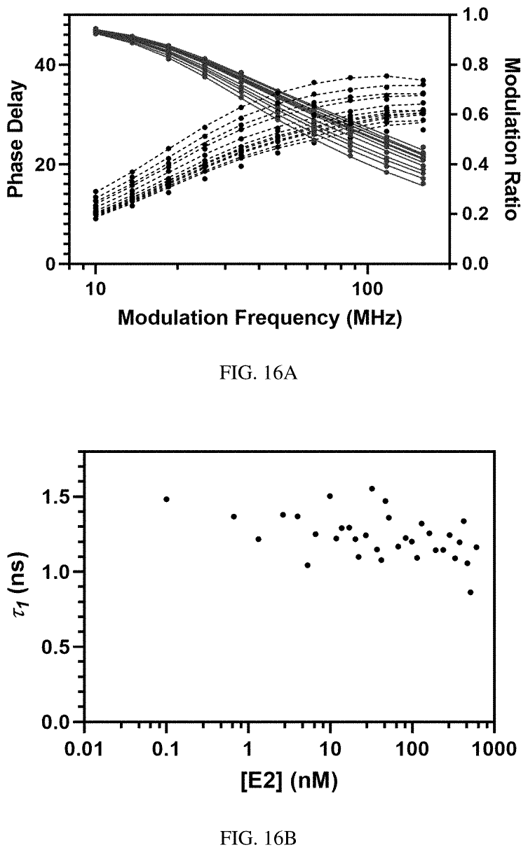

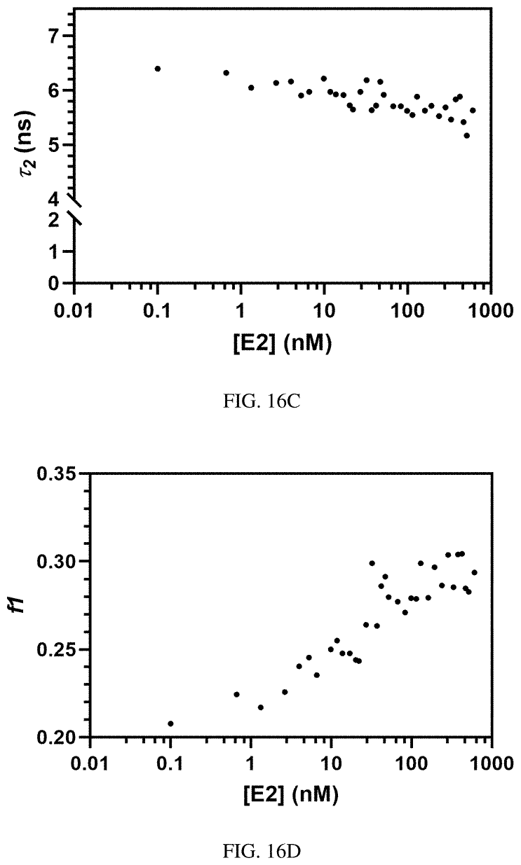

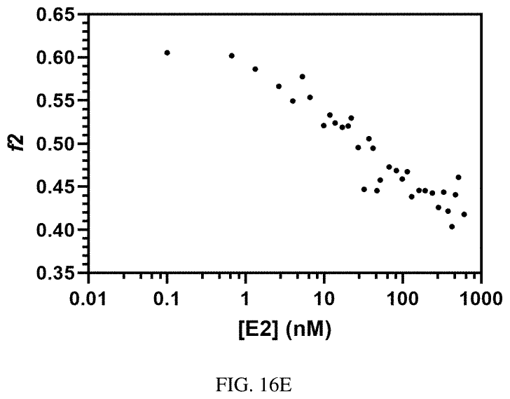

In the absence of SHBG, bis-ANS was efficiently quenched in the aqueous environment, with a singlet-excited state lifetime of approximately 250 ps and low quantum yield. Once partitioned in the interior of a protein, bis-ANS molecules display longer lifetimes in the range of 5 to 8 ns with concomitant increase in fluorescence emission. The excited state emission data fit to two decays (FIG. 16) corresponding to 1.2 ns and 6.7 ns with ×2 between 1 and 2.3. The fractional intensities of the short- and long-lifetime components were found to be sensitive to estradiol concentrations reflecting the E2-induced global conformational rearrangement and repartitioning of bis-ANS within the SHBG dimer upon ligand binding.

FIG. 16A shows the phase and frequency modulation data as graded concentrations of estradiol were added to a solution containing SHBG and bis-ANS. The titration of estradiol into an SHBG solution led to concentration-dependent changes in bis-ANS partitioning. Alterations in integrated fluorescence from bis-ANS could result either from repartitioning of the hydrophobic probe in the protein interior or from changes in fluorescence lifetimes. Accordingly, the phase and frequency modulation data were analyzed to determine the fractional contributions of free and bound bis-ANS and the singlet excited state lifetimes. Estradiol titration into SHBG exerted only a minimal effect on the excited state lifetimes of the bound and free bis-ANS (FIGS. 16B and 16C). However, as the estradiol concentration was increased, the fractional contribution from the short-lifetime substantially increased and that of the long-lifetime component decreased (FIGS. 16D and 16E), indicating that estradiol binding to SHBG leads to global conformational changes in SHBG, stabilizing overall structure and expelling bis-ANS from the protein interior.

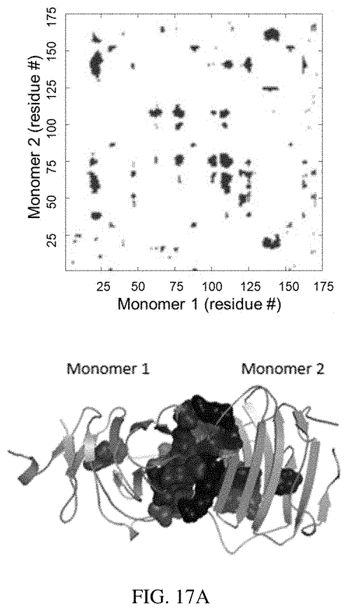

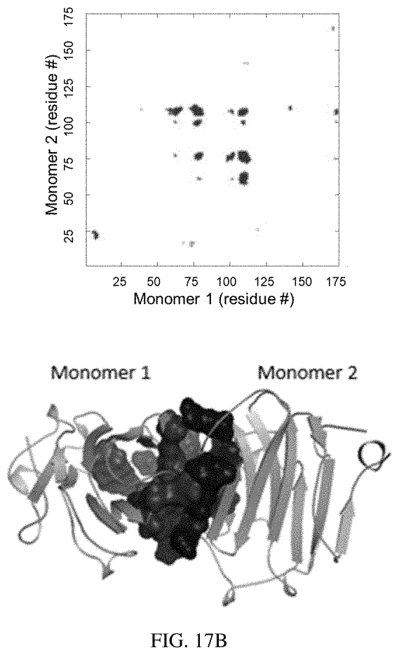

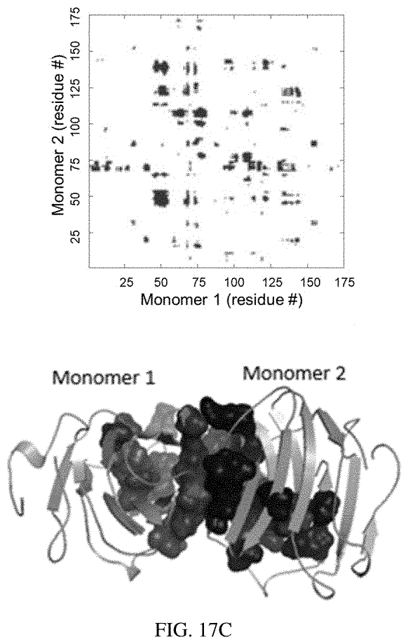

Dynamic cross-correlation matrix analysis shows allosteric changes in residue correlations upon estradiol binding to either of the two monomers. To analyze the effect of the estradiol binding on the residue rearrangement in each of the two monomers in the three states (unliganded, FIG. 17A; singly bound, FIG. 17B and doubly bound, FIG. 17C), dynamic cross-correlation matrices (DCCM) were generated for each residue in the two monomers. The right panels for each of the DCCM plots show the spatial location of residues, which exhibit correlated motions at the dimerization interface and in the ligand binding pockets across two monomers in each of the states. Contrary to what would be predicted by the prevailing linear model of estradiol binding to SHBG, DCCM plots show that residue correlations in monomers within the unliganded dimer are not identical and that each monomer samples distinct conformational states even in the absence of estradiol. The binding of the first estradiol molecule to monomer 1 resulted in changes in correlated motions in residues of both monomers 1 and 2 (FIG. 17B). Similarly, the binding of estradiol to monomer 2 resulted in substantial changes in the coupling of motions in the residues of estradiol-bound monomer 1 (FIG. 17C). Collectively, the occurrence of several new correlations within each monomer when estradiol binds to either of the two monomers provides evidence of an allosterically-coupled network of residues within the two monomers of SHBG.

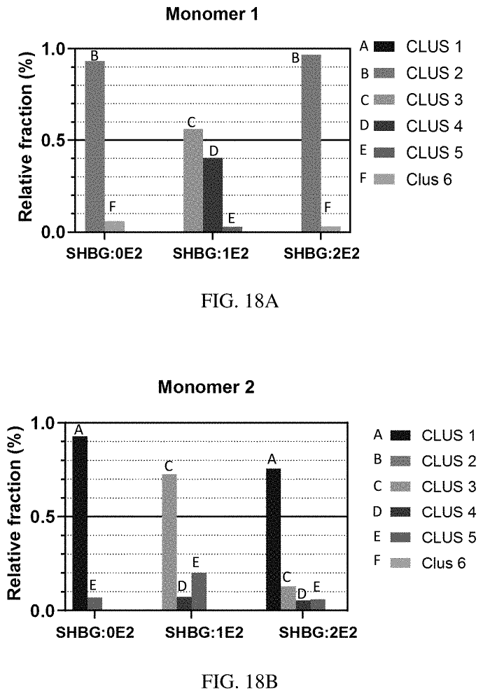

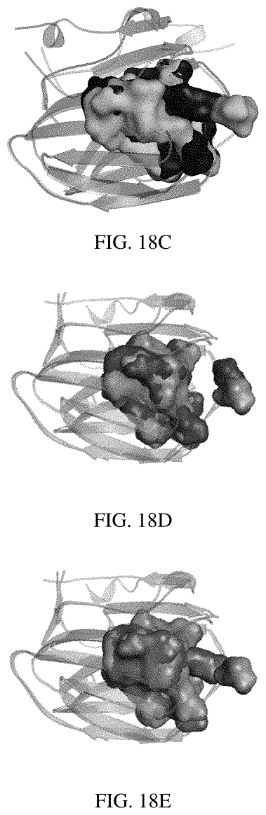

Markov state models reveal dynamic allosteric conformational coupling between the SHBG monomers. Markov state models have been used to study the protein conformational transitions in ligand-bound and unbound states and to estimate the state composition from population-averaged residue trajectory data [47A-51A]. The Markovian framework was utilized to examine all-atom molecular modeling (MD) trajectories and determine the estradiol-induced redistribution of SHBG monomers among various conformational ensembles and their temporal distribution in microstates after estradiol binding. Inter-monomeric allostery within the SHBG dimer was studied using AMBER suite of programs to generate conformational clusters based on pairwise RMSD measures using a k-means procedure. Spanning the project space for the possibility of 2-12 clusters, the most parsimonious distribution of clusters was found to be 6 (FIG. 18, clusters C1-C6). Distribution functions of the distances of the SHBG microstates with respect to each cluster centroid were calculated for the six clusters.

FIG. 18 shows that the two monomers populate the six conformational clusters to varying degree depending on the estradiol occupancy. Even in the unliganded state, the monomers populate conformationally distinct clusters and are not equivalent (FIGS. 18A, 18B, and 18C). When neither of the two monomers is occupied by the ligand, monomer 1 is distributed predominantly between conformational clusters C2 and C6, but monomer 2 is distributed predominantly in clusters C1 and C5 (FIGS. 18A and 18B; SHBG:0E2). Notably, binding of the first estradiol molecule not only alters the conformational ensemble of monomer 1 but also changes the distribution of monomer 2 in clusters C3, C4 and C5, providing evidence of inter-monomeric allostery (FIGS. 18A and 18B; SHBG:1E2). When the second estradiol molecule binds to monomer 2, it not only influences populations of conformational states of monomer 2 but also changes the distribution of the conformational states of the already occupied monomer 1 (FIGS. 18A and 18B; SHBG:2E2).

To determine if the orientation of the binding pocket residues differed among the clusters, the overlay of the cluster pairs was generated (FIGS. 18C, 18D, and 18E), which were predominantly populated by the two monomers in the three states (SHBG:0E2, SHBG:1E2 and SHBG:2E2). The conformational states populated by the monomers in these clusters are substantially different both in the ligand binding pockets and in the residues distant from the ligand binding pocket. The relative probability of partitioning of monomers in distinct conformational clusters and the associated transition rates were used to elucidate the energy landscapes for each monomer.

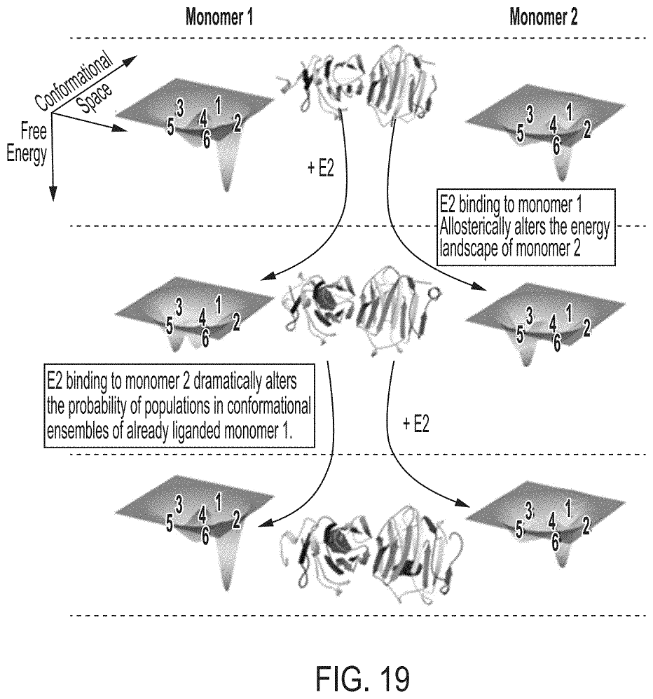

The Markov state model (MSM) analyses revealed that even in the absence of estradiol, the two monomers within the SHBG dimer exhibit distinct energy landscapes (FIG. 19) and that estradiol binding is associated with dynamic redistribution of ensemble populations. FIG. 19A shows that the unliganded SHBG monomers occupy distinct energy landscapes consistent with their conformational heterogeneity in the unliganded state. Upon binding of estradiol to the first monomer, the populations of energy states occupied by each of the two monomers are altered (FIG. 19B). The relative probabilities for occupancy of conformational clusters for both the monomers are sensitive to the occupancy of either of the two monomers (FIG. 19B). Similarly, the energy landscape of the estradiol-bound first monomer is subsequently altered by estradiol's binding to the second monomer.

These data demonstrate that estradiol binding to either of the monomers alters the conformational states and energy landscapes of both monomers. The net effect of the perturbations associated with estradiol binding is the reshaping of the free-energy landscape of each monomer and to stabilize distinct conformation states. Such dynamic redistribution of SHBG conformational ensemble into clusters could explain the continuum of apparent affinity of estradiol for SHBG dimer, depending on the relative concentrations of E2 and SHBG, observed in the equilibrium dialysis and optical spectroscopy experiments.

### Discussion

These results show the allosteric interaction between the two monomers upon estradiol's binding to SHBG. First, the binding isotherms of estradiol's association with SHBG generated using equilibrium dialysis and steady-state fluorescence spectroscopy are neither linear nor symmetric around the point corresponding to 50% bound estradiol concentration; the complex asymmetry of the binding isotherm is inconsistent with the notion of homogeneous binding of estradiol to SHBG with a single Kd. The binding isotherms also do not conform to two rigid binding sites, each with a fixed but distinct Kd. Second, the “apparent” Kd varied substantially as the concentrations of estradiol and SHBG as well as the ratios of their relative concentrations were varied. Because the primary amino acid structure of the two monomers within each SHBG dimer is identical, the dynamic concentration-dependent variation in the “apparent” Kd can only be explained by a dynamic intra-molecular conformational rearrangement within the SHBG dimer upon ligand binding that alters the Kd of the ligand binding pocket. Third, when bis-ANS probe was used to examine its partitioning into the interior of SHBG, the fractional intensities of its short- and long-lifetime components were found to be highly sensitive to estradiol concentrations. With increasing estradiol concentrations, the short-lifetime fraction increased, suggesting a more compact SHBG structure in the ligand-bound state. The estradiol-induced repartitioning of the bis-ANS probe within SHBG provides direct experimental evidence of a global conformational rearrangement in SHBG upon estradiol binding. Finally, the molecular modeling simulations reveal that estradiol binding to monomer 1 induces conformational rearrangements in the estradiol binding pocket of both monomers. Thus, the molecular modeling studies provide support for the allosteric interaction between the two monomers upon ligand binding.

Markov state models developed using the molecular dynamics trajectories provide a detailed understanding of the molecular processes involved in the allosteric coupling between SHBG monomers. The conformational clustering analysis from states-and-rates network models illustrate that binding of first estradiol molecule significantly alters the probabilities of the distribution of both monomers in various conformational states as well as the associated energy landscapes of both monomers. Interestingly, binding of the second estradiol molecule to monomer 2 also impacts the landscape and probability of conformational transitions in estradiol-bound monomer 1. The Markov state models show that allosteric coupling in the SHBG monomers changes the energy landscape such that the two monomers are not equivalent even in the fully bound state. This finding helps explain difficulties in resolution of the unliganded SHBG and why only one of the monomers within the SHBG dimer could be crystallized [18A].

The results for estradiol binding to SHBG are consistent with the evidence of ensemble allostery in testosterone's binding to SHBG [53A]. The ligand-induced allosteric interaction between the monomers may be a more general mechanism among multimeric binding proteins.

It is possible that allostery extends the range of ligand concentrations that the binding protein can bind; furthermore, it may offer a mechanism to regulate the amount of available free ligand depending upon the prevalent ligand concentration. Thus, at a low partial pressure of oxygen, a relatively greater fraction of oxygen remains unbound to hemoglobin, while at higher partial pressures of oxygen, more oxygen becomes bound. Analogously, estradiol concentrations vary from low levels in pre-pubertal and menopausal women (2 to 6 pg/mL) to levels as high as 30,000 to 40,000 pg/mL during pregnancy. The nonlinear dynamics of estradiol's binding and the allosteric coupling of monomers within SHBG provides a mechanism for extending the range of estradiol binding and offer a potential mechanism for facile regulation of free hormone bioavailability as estradiol concentrations vary widely during different phases of the reproductive and nonreproductive phases of a person's life.

### REFERENCES

1A. Anderson D. Sex-Hormone-Binding Globulin. Clinical Endocrinology. 1974; 3:69-96.
2A. Dunn J F, Nisula B C, Rodbard D. Transport of Steroid Hormones: Binding of 21 Endogenous Steroids to Both Testosterone-Binding Globulin and Corticosteroid-Binding Globulin in Human Plasma. J Clinc Endocrinol Metab. 1981; 53(1):58-68.
3A. Moll G W, Rosenfield R L, Helke J H. Estradiol-Testosterone Binding Interactions and Free Plasma Estradiol under Physiological Conditions. J Clin Endocrinol Metab. 1981; 52(5):868-874.
4A. Tietz N W, ed. Textbook of Clinical Chemistry. Philadelphia, Pa.: Saunders; 1986.
5A. Peters T Jr. All About Albumin: Biochemistry, Genetics, and Medical Applications. San Diego, Calif.: Academic Press; 1996.
6A. Pearlman W H, Fong IFF, Tou J H. A further study of a testosterone-binding component of human pregnancy plasma. J Biol Chem. 1969; 244:1373.
7A. Burke C W, Anderson D C. Sex-Hormone-Binding Globulin is an Oestrogen Amplifier. Nature. 1972; 240
8A. Vigersky R A, Kono S, Sauer M, Lipsett M B, Loriaux D L. Relative Binding of Testosterone and Estradiol to Testosterone-Estradiol-Binding Globulin. J Clin Endocrinol Metab. 1979; 49(6):899-904.
9A. Goldman A L, Bhasin S, Wu F C, Krishna M, Matsumoto A M, Jasuja R. A Reappraisal of Testosterone's Binding in Circulation: Physiological and Clinical Implications. Endocrine Reviews. 2017; 38:302-324.
10A. Rosner W, Smith R N. Isolation and characterization of the testosterone-estradiol binding globulin from human plasma. Use of a novel affinity column. Biochemistry. 1975; 14(22):4813-20.
11A. Manni A, Pardridge W M, Cefalu W, Nisula B C, Bardin C W, Santner S J, Santen R J. Bioavailability of albumin-bound testosterone. J Clin Endocrinol Metab. 1985; 61(4):705-710.
12A. Nisula B C, Dunn J F. Measurement of the testosterone binding parameters for both testosterone-estradiol binding globulin and albumin in individual serum samples. Steroids. 1979; 34(7):771-791.
13A. Murphy B E. Binding of testosterone and estradiol in plasma. Can J Biochem. 1968; 46(4):299-302.
14A. Zeginiadou T, Kolias S, Kouretas D, Antonoglou O. Nonlinear binding of sex steroids to albumin and sex hormone binding globulin. Eur J Drug Metab Pharmacokin. 1997; 22(3):229-235.
15A. Laurent M R, Hammond G., Blokland M. et al. Sex hormone-binding globulin regulation of androgen bioactivity in vivo: validation of the free hormone hypothesis. Sci Rep 6, 35539 (2016)
16A. Laurent M R, and Vanderschueren, D. “Functional effects of sex hormone-binding globulin variants.” Nature Reviews Endocrinology, vol. 10, no. 9, 2014
17A. Grishkovskaya I, Avvakumov G V, Sklenar G, Dales D, Hammond G L, Muller Y A. Crystal Structure of Human Sex Hormone-Binding Globulin: Steroid Transport by a Laminin G-Like Domain. EMBO J. 2000; 19(4):504-512.
18A. Grishkovskaya I, G Sklenar, G V Avvakumov, D Dales, J Behlke, G L Hammond, Y A Muller. Crystallization of the N-terminal domain of human sex hormone-binding globulin, the major sex steroid carrier in blood. Acta Crystallographica Section D. 1999; 55:2053-2055.
19A. Avvakumov G, Grishkovskaya I, Muller Y, Hammond G. Resolution of the Human Sex Hormone-binding Globulin Dimer Interface and Evidence for Two Steroid-binding Sites per Homodimer. J Biol Chem. 2001:34453-34457.
20A. Sodergard R, Backstrom T, Shanbhag V, Carstensen H. Calculation of Free and Bound Fractions of Testosterone and Estradiol-17β to Human Plasma Proteins at Body Temperature. J Steroid Biochem. 1982; 16:801-810.
21A. Vermeulen A, Verdonck L, Kaufman J M. A Critical Evaluation of Simple Methods for the Estimation of Free Testosterone in Serum. The J of Clinc Endocrinol & Metab. 1999; 84(10):3666-3672.
22A. Grishkovskaya, G V Avvakumov, G L Hammond, M G Catalano, Y A Muller. Steroid Ligands Bind Human Sex Hormone-binding globulin in Specific Orientations and Produce Distinct Changes in Protein Conformation. J Biol Chem. 2002; 277(5):32086-32093.
23A. Mazer N A. A novel spreadsheet method for calculating the free serum concentrations of testosterone, dihydrotestosterone, estradiol, estrone, and cortsiol: With illustrative examples from male and female populations. Steroids. 2009; 74:512-519.
24A. Sui L M, Hughes W, Agnes J H, Petra P. Direct evidence for the localization of the steroid-binding site of the plasma sex steroid-binding protein (SBP or SHBG) at the interface between the subunits. Protein Science. 1996; 5:2514-2520.
25A. Petra P, Adman E, Orr W, Woodcock K, Groff C, Sui L M. Arginine-140 and isoleucine-141 determine the 17β-estradiol binding specificity of the sex-steroid binding protein (SBP, or SHBG) of human plasma. Protein Science. 2001; 10:1811-1821.
26A. Wu, C H; Motohashi, T; Abdel-Rahman, H A; Flickinger, G L; Mikhail, G. Free and protein-bound plasma estradiol-17 beta during the menstrual cycle. J Clin Endocrinol Metab, 1976; 43(2), 436-445
27A. Orwoll E, Lambert L C, Marshall L M, et al. Testosterone and Estradiol among Older Men. J Clin Endocrinol Metab. 2006; 91(4).
28A. Baird D T, Guevara A. Concentration of unconjugated estrone and estradiol in peripheral plasma in nonpregnant women throughout the menstrual cycle, castrated and postmenopausal women and in men. J Clin Endocrinol Metab. 1969; 29(2)
29A. Gasteiger E, Hoogland C, Gattiker A, et al. Protein Identification and Analysis Tools on the ExPASy Server: Humana Press; 2005.
30A. Jasuja G K, Travison T G, Davda M, et al. Age Trends in Estradiol and Estrone Levels Measured Using Liquid Chromatography Tandem Mass Spectrometry in Community-Dwelling Men of the Framingham Heart Study. The J of Gerontol: Medical Sciences Series A. 2013; 68(6):733-740.
31A. Snyder P J, Bhasin S, Cunningham G R, Matsumoto A M, Stephens-Shields A J, Cauley J A, Gill T M, Barrett-Connor E, Swerdloff R S, Wang C, Ensrud K E, Lewis C E, Farrar J T, Cella D, Rosen R C, Pahor M, Crandall J P, Moltich M E, Cifelli D, Dougar D, Fluharty L, Resnick S M, Storer T W, Anton S, Basaria S, Diem S J, Hou X, Mohler E R III, Parsons J K, Wenger N K, Zeldow B, Landis J R, Ellenberg S S. Effects of Testosterone Treatment in Older Men. N Engl J Med. 2016; 374(7):611-624.
32A. Zakharov M N, Ulloor J, Bhasin S, et al. Guanidinium chloride-induced spectral perturbations of 4,4′-dianilino-1,1′-binaphthyl-5,5′-disulfonic acid confound interpretation of data on molten globule states. Anal Biochem. 2011; 416(1):126-128.
33A. Grishkovskaya, I., Avvakumov, G. V, Hammond, G. L., Catalano, M. G. & Muller, Y. A. Steroid ligands bind human sex hormone-binding globulin in specific orientations and produce distinct changes in protein conformation. J. Biol. Chem. 277, 32086-32093 (2002).
34A. UniProt: the universal protein knowledgebase. Nucleic Acids Res. 45, D158-D169 (2017).
35A. Gordon, J. C. et al. H++: a server for estimating p K as and adding missing hydrogens to macromolecules. Nucleic Acids Res. 33, W368-W371 (2005).
36A. Maier, J. A. et al. ff14S B: improving the accuracy of protein side chain and backbone parameters from ff99S B. J. Chem. Theory Comput. 11, 3696-3713 (2015).
37A. Jorgensen, W. L., Chandrasekhar, J., Madura, J. D., Impey, R. W. & Klein, M. L. Comparison of simple potential functions for simulating liquid water. J. Chem. Phys. 79, 926-935 (1983).
38A. Berendsen, H. J. C., Postma, J. P. M. van, van Gunsteren, W. F., DiNola, A. & Haak, J. R. Molecular dynamics with coupling to an external bath. J. Chem. Phys. 81, 3684-3690 (1984).
39A. Ryckaert, J.-P., Ciccotti, G. & Berendsen, H. J. C. Numerical integration of the cartesian equations of motion of a system with constraints: molecular dynamics of n-alkanes. J. Comput. Phys. 23, 327-341 (1977).
40A. Wu, X. & Brooks, B. R. Self-guided Langevin dynamics simulation method. Chem. Phys. Lett. 381, 512-518 (2003).
41A. Case, D. A. et al. AMBER 2018; 2018. Univ. California, San Fr.
42A. Gotz, A. W. et al. Routine microsecond molecular dynamics simulations with AMBER on GPUs. 1. Generalized born. J. Chem. Theory Comput. 8, 1542-1555 (2012).
43A. Salomon-Ferrer, R., Götz, A. W., Poole, D., Le Grand, S. & Walker, R. C. Routine microsecond molecular dynamics simulations with AMBER on GPUs. 2. Explicit solvent particle mesh Ewald. J. Chem. Theory Comput. 9, 3878-3888 (2013).
44A. Roe, D. R. & Cheatham III, T. E. PTRAJ and CPPTRAJ: software for processing and analysis of molecular dynamics trajectory data. J. Chem. Theory Comput. 9, 3084-3095 (2013).
45A. Humphrey, W., Dalke, A. & Schulten, K. VMD: visual molecular dynamics. J. Mol. Graph. 14, 33-38 (1996).
46A. DeLano, W. L. Pymol: An open-source molecular graphics tool. CCP4 Newsl. protein Crystallogr. 40, 82-92 (2002).
47A. Shukla, D.; Hernandez, C. X.; Weber, J. K.; Pande, V. S. Markov State Models Provide Insights into Dynamic Modulation of Protein Function. Acc. Chem. Res. 2015, 48, 414-422.
48A. Chodera, J. D.; Noe, F. Markov State Models of Biomolecular Conformational Dynamics. Curr. Opin. Struct. Biol. 2014, 25, 135-144.
49A. Schwantes, C. R.; McGibbon, R. T.; Pande, V. S. Perspective: Markov Models for Long-Timescale Biomolecular Dynamics. J. Chem. Phys. 2014, 141, 090901-090907.
50A. Prinz, J.-H.; Keller, B.; Noe, F. Probing Molecular Kinetics with Markov Models: Metastable States, Transition Pathways and Spectroscopic Observables. Phys. Chem. Chem. Phys. 2011, 13, 16912-16927.
51A. K M Thayer, B Lakhani, D L Beveridge. Molecular Dynamics-Markov State Model of Protein Ligand Binding and Allostery in CRIB-PDZ: Conformational Selection and Induced Fit. J Phys Chem B. 2017; 8:5509-5514.
52A. Heinrich-Balard L, Zeinyeh W, Déchaud H, et al. Inverse Relationship between hSHBG affinity for testosterone and hSHBG concentration revealed by surface plasmon resonance. Mol and Cell Endocrinol. 2014; 399:201-207.
53A. Zakharov M N, Bhasin S, Travison T G, Xue R, Ulloor J, Vasan R S, Carter E C, Wu F, Jasuja R. A multi-step, dynamic allosteric model of testosterone's binding to sex hormone binding globulin. Mol Cell Endocrinol. 2015; 399:190-200.
54A. J Metzger, A Schnitzbauer, M Meyer, M Soder, C Y Cuilleron, H Hautpmann, E Huber, P B Luppa. Binding Analysis if 1-alpha- and 17-alpha-Dihydrotestosterone Derivatives to Homodimeric Sex Hormone-Binding Globulin. Biochem. 2003; 42:13735-13745.
55A. Perutz M F. Stereochemistry of Cooperative Effects in Haemoglobin: Haem-Haem Interaction and the Problem of Allostery. Nature. 1970; 228:726-734.
56A. Monod, J.; Wyman, J. C. J.; Changeux, J-P. On the Nature of Allosteric Transitions: A Plausible Model. J. Mol. Biol. 1965, 12, 88-118.
57A. Koshland, D. E.; Nemethy, G.; Filmer, D. Comparison of Experimental Binding Data and Theoretical Models in Proteins Containing Subunits. Biochemistry 1966, 5, 365-385.
58A. Hilser, V. J.; Wrabl, J. O.; Motlagh, H. N. Structural and Energetic Basis of Allostery. Annu. Rev. Biophys. 2012, 41, 585-609.
59A. Motlagh, H. N.; Li, J.; Thompson, E. B.; Hilser, V. J. Interplay between Allostery and Intrinsic Disorder in an Ensemble. Biochem. Soc. Trans. 2012, 40, 975-980.

What is claimed herein is:

1. A method of determining a free sex steroid concentration in a biological sample comprising the following steps:
a) identifying in the biological sample
i) a total SHBG concentration,
ii) a total sex steroid concentration, and
iii) an albumin concentration;
b) attributing at least two distinct interconverting microstates of an unliganded SHBG dimer having a first monomer and a second monomer;
c) calculating the free sex steroid concentration in the biological sample using an ensemble allostery model encompassing readjustment of a first equilibria between the microstates upon binding of a first sex steroid molecule to the first monomer and an allosteric interaction between two binding sites of the SHBG dimer.

2. The method of claim 1, wherein the sex steroid is selected form the group consisting of a testosterone steroid, an estradiol steroid, and estrone steroid, and a dihydrotestosterone steroid.

3. A method of diagnosing and treating a sex steroid disorder in a patient comprising the following steps:
a) obtaining a biological sample from the patient,
b) measuring in the biological sample obtained in step a)
i. a total concentration of sex-hormone binding globulin (“SHBG”), which is a dimer having a first monomer and a second monomer,
ii. a total concentration of the sex steroid, and
iii. a concentration of albumin;
c) determining the concentration of free sex steroid in the biological sample based on (i)-(iii) measured in step b), using an implementation of ensemble allostery model representing the binding equilibria (i) between the sex steroid and the SHBG dimer first monomer of the SHBG and between the sex steroid and the second monomer of the SHGB, wherein the unliganded SHBG has at least two distinct interconverting microstates, and wherein the first monomer and the second monomer have an allosteric interaction such that each of the microstates binds a first sex steroid molecule with a different affinity; and (ii) between the sex steroid and the albumin;
d) diagnosing the patient with the sex steroid disorder when the free sex steroid concentration determined in step c) is below the lower limit of a normal free sex steroid concentration from a healthy individual; and
e) administering an effective amount of sex steroid, sex steroid derivatives, and/or analogues thereof to the patient diagnosed in step d).

4. The method of claim 3, wherein the sex steroid is selected form the group consisting of a testosterone steroid, an estradiol steroid, and estrone steroid, and a dihydrotestosterone steroid.

5. The method of claim 3, wherein the step of measuring in the biological sample further comprises measuring in the biological sample
iv. A concentration of at least a second sex steroid wherein the second sex steroid is different than the sex steroid that was measured in step (b).

6. The method of claim 3, wherein the step of measuring in the biological sample further comprises measuring in the biological sample
iv. the concentration of one or more analytes.

7. The method of claim 3, wherein the step of measuring in the biological sample further comprises measuring one or more of the concentrations in the biological sample using at least one analytical method.

8. The method of claim 7, wherein the analytical method is selected from the group consisting of an immunoassay and a mass spectrometry-based assay.

9. The method of claim 3, wherein the sex steroid disorder comprises an estrogen deficiency.

10. The method of claim 3, wherein the sex steroid disorder comprises an estrogen excess.

11. The method of claim 3, wherein treatment further comprises the step of:
d) adjusting the dose of administered sex steroid, sex steroid derivatives, and/or analogues thereof for treatment of the sex steroid disorder.

12. The method of claim 5, wherein the method further comprises the step of determining the concentration of the second sex steroid in the biological sample using an implementation of ensemble allostery model comprising readjusting a second equilibria between the microstates upon binding of the second sex steroid molecule to the first monomer and the allosteric interaction between two monomers of the SHBG.

13. A method of monitoring and optimizing treatment for a sex steroid deficiency in a patient comprising the following steps:
a) obtaining a biological sample from the patient who is on a treatment of the sex steroid deficiency,
b) measuring in the biological sample obtained in step a)
i. a total concentration of sex-hormone binding globulin (“SHBG”), which is a dimer having a first monomer and a second monomer,
ii. a total concentration of the sex steroid, and
iii. a concentration of albumin;
c) determining the concentration of free sex steroid in the biological sample based on (i)-(iii) measured in step b), using an implementation of ensemble allostery model representing the binding equilibria (i) between the sex steroid and the first monomer of the SHBG and between the sex steroid and the second monomer of the SHBG, wherein the unliganded SHBG has at least two distinct interconverting microstates and wherein the first monomer and the second monomer have an allosteric interaction such that each of the microstates binds the sex steroid molecule with a different affinity; and (ii) between the sex steroid and the albumin;
d) identifying the patient as needing treatment optimization when the free sex steroid concentration determined in step c) is below the lower limit of a normal free sex steroid concentration from a healthy individual; and
e) optimizing the treatment of the identified patient with the sex steroid disorder by administering a modified amount of the sex steroid, sex steroid derivatives, and/or analogues thereof to the identified patient.

14. The method of claim 13, wherein the sex steroid is selected form the group consisting of a testosterone steroid, an estradiol steroid, and estrone steroid, and a dihydrotestosterone steroid.

15. A method of treating a sex steroid disorder in a patient comprising administering an effective amount of sex steroid, sex steroid derivatives, and/or analogues thereof to the patient wherein the patient's free sex steroid concentration is below the lower limit of a normal free sex steroid concentration from a healthy individual, wherein the patient's free sex steroid concentration is determined based on:
i. total concentration of sex-hormone binding globulin (“SHBG”), which is a dimer having a first monomer and a second monomer,
ii. total concentration of the sex steroid, and
iii. concentration of albumin;
and wherein the free sex steroid concentration is calculated by an implementation of ensemble allostery model representing the binding equilibria (i) between the sex steroid and the first monomer of the SHBG and between the sex steroid and the second monomer of the SHGB, wherein the unliganded SHBG has at least two distinct interconverting microstates, and wherein the first monomer and the second monomer have an allosteric interaction such that each of the microstates binds a first sex steroid molecule with a different affinity; and (ii) between the sex steroid and the albumin.

16. The method of claim 15, wherein the sex steroid is selected form the group consisting of a testosterone steroid, an estradiol steroid, and estrone steroid, and a dihydrotestosterone steroid.
